(19) United States
(12) Patent Application Publication
(10) Pub. No.: US 2024/0366662 A1
(43) Pub. Date: Nov. 7, 2024

(54) CAR T CELL THERAPY AND IFN GAMMA

(71) Applicant: CITY OF HOPE, Duarte, CA (US)

(72) Inventors: Darya Alizadeh, Duarte, CA (US); Christine E. Brown, Duarte, CA (US)

(21) Appl. No.: 18/284,413

(22) PCT Filed: Mar. 30, 2022
(86) PCT No.: PCT/US2022/022575

§ 371 Date: Sep. 27, 2023

Related U.S. Application Data
(60) Provisional application No. 63/168,210, filed on Mar. 30, 2021.

(51) Int. Cl. A61K 35/17; A61K 39/00; A61P 35/00; C07K 14/57; C07K 14/725; C07K 16/28; C12N 5/0783

(52) U.S. Cl. CPC A61K 35/17; A61K 39/4611; A61K 39/4631; A61K 39/464419; A61K 39/46444; A61K 39/464441; A61P 35/00; C07K 14/57; C07K 14/7051; C07K 16/2866; C12N 5/0636; A61K 2239/13; A61K 2239/21; A61K 2239/28; A61K 2239/47; A61K 2239/57; C07K 2317/622; C07K 2319/02; C07K 2319/03; C12N 2510/00

(57) ABSTRACT

Provided herein are, inter alia, compositions comprising chimeric antigen receptor (CAR)-engineered immune cells, methods of formulating, and methods useful for treating cancer and leukemia.

### CLAIM OF PRIORITY

This application claims the benefit of U.S. Provisional Application Ser. No. 63/168,210, filed on Mar. 30, 2021. The entire contents of the foregoing are incorporated herein by reference.

### TECHNICAL FIELD

This disclosure concerns chimeric antigen receptor (CAR)-engineered immune cells, methods of formulating, and methods of use.

### BACKGROUND

Glioblastoma (GBM) is among the deadliest cancers with very limited therapeutic options (1, 2, 3). Despite aggressive standard-of-care therapies, tumor recurrence is almost inevitable and uniformly lethal, with most patients not surviving beyond two years from diagnosis. Advances in immunotherapy have inspired efforts to develop therapeutic strategies for eliciting anti-tumor immune responses in GBM, including adoptive transfer of chimeric antigen receptor (CAR) T cells. Clinical studies evaluating CAR T cells in GBM have demonstrated early evidence of safety and bioactivity in selected patients, nevertheless, the responses have been limited. Challenges for productive CAR T cell therapy for solid tumors such as GBM are multifactorial. Tumor heterogeneity and cellular plasticity allows for outgrowth of antigen loss tumor variants, leading to treatment failure. The tumor microenvironment, for GBM tumors, are myeloid-rich with scant T cell population, which also poses specific challenges to CAR T cells.

IL13Rα2-CAR T therapy has shown some promise in treating GBM despite the non-uniform expression of IL13Rα2 by tumor cells (4). The response was associated with increase in CNS inflammatory cytokines and infiltration of endogenous immune cells (4). In line with this observation, a recent longitudinal analysis of immune-monitoring after HER2-CAR T cell therapy showed evidence of endogenous immune reactivity which may have contributed to the patient's favorable response (5).

Pro-inflammatory cytokines secreted by CAR T cells, such as IFNγ, may play an important role in activation and programming of the immune infiltrates in GBM TME. IFNγ can activate macrophage (6) and microglia (7), recruit and activate cytotoxic T cells, polarize CD4+ T cells into Th1 effector cells and impair tumor-promoting Treg development and function (8, 9, 10). IFNs can additionally act as a key signal (30) to facilitate the activation and priming of tumor reactive T cells (11).

### SUMMARY

Described herein are immune system cells, e.g., T cells or NK cells, that express both a CAR targeted to a tumor antigen and human IFNγ that is encoded by a nucleic acid molecule (“recombinant human IFNγ”), e.g., immune cells harboring a nucleic acid molecule that encodes both a CAR and human IFNγ. Without being bound by any theory it appears that the co-expression increases one or more of activation of the immune cells, proliferation of the immune cells and tumor cell killing by endogenous cells that recognize tumor cells. The CAR can include a targeting domain that is an scFv targeted to a tumor antigen (e.g., an scFv targeted to CD19) or a ligand (e.g., IL-13 or a variant thereof) that binds a receptor on tumor cells. Thus, the cells can harbor a nucleic acid molecule that encodes a CAR and human IFNγ. Expression of the CAR and the human IFNγ can be under the control of the same expression control sequences or under the control of different expression control sequences. The cells can harbor a nucleic acid molecule that encodes a single amino acid sequence that includes a CAR and human interferon gamma. For example, the amino acid sequence of the CAR can be followed by a ribosomal skip sequence and then an amino acid sequence that includes human IFNγ. The amino acid sequence can include at least one signal sequence for secretion of a protein (e.g., a signal sequence for secretion of the CAR and a signal sequence for expression of the human IFNγ). In some embodiments, a nucleic acid of the disclosure can be a non-endogenous nucleic acid.

Immune cells that express a CAR and interferon can target and kill cancer cells expressing the target of the CAR. In addition, they can activate killing of cancer cells that do not express the express the target of the CAR by, for example, activating innate and adaptive immune subsets in tumor microenvironment. In this manner, they are useful for treating tumors that include both cancer cells expressing the target of the CAR and cancer cells that do not express the target of the CAR or have very low expression of the target of the CAR.

The human IFNγ can comprise the following amino acid sequence:

(SEQ ID NO: 1)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQMLFRGRRASQ

The human IFN-γ amino acid sequence can be preceded by a signal sequence that directs secretion of the human interferon gamma from a eukaryotic cell, e.g., a human cell. Thus, human interferon gamma precursor can be used (signal sequence underlined):

(SEQ ID NO: B)
MKYTSYILAFQLCIVLGSLGCYCQDPYVKEAENLKKYFNAGHSDVADNGT
LFLGILKNWKEESDRKIMQSQIVSFYFKLFKNFKDDQSIQKSVETIKEDM
NVKFFNSNKKKRDDFEKLTNYSVTDLNVQRKAIHELIQVMAELSPAAKTG
KRKRSQMLFRGRRASQ

The CAR can be targeted to a tumor antigen, not limiting examples of which include:

A suitable IL-13 CAR comprises a variant of human IL-13 comprising the following amino acid sequence:

(SEQ ID NO: C)
GPGPVPPSTALRYLIEELVNITQNQKAPLCNGSMVWSINLTAGMYCAALE
SLINVSGCSAIEKTQRMLSGFCPHKVSAGQFSSLHVRDTKIEVAQFVKDL
LLLHLKKLFREGREN

Sequence of wild-type human IL13 (signal sequence underlined):

(SEQ ID NO: D)
MHPLLNPLLLALGLMALLLTTVIALTCLGGFASPGPVPPSTALRELIEEL
VNITQNQKAPLCNGSMVWSINLTAGMYCAALESLINVSGCSAIEKTQRML
SGFCPHKVSAGQFSSLHVRDTKIEVAQFVKDLLLHLKKLFREGREN

The IL-13 CAR can include a variant IL13 comprising, for example, SEQ ID NO:C; a spacer (e.g., comprising any of SEQ ID NOs: 2-12); a transmembrane domain (e.g., comprising any of SEQ ID NOs: 13-20); a co-stimulatory domain (comprising any of SEQ ID NOs: 22-25); optionally a linker of 3-15 amino acids (e.g., GGG); and a CD3 zeta cytoplasmic domain (SEQ ID NO: 21 or a variant thereof comprising any of SEQ ID NOs: 50-56). A useful CAR can comprise any of SEQ ID NO: 70-76.

GPVPPSTALRYLIEELVNITQNQKAPLCNGSMVWSINLTAGMYCAALESL
INVSGCSAIEKTQRMLSGFCPHKVSAGQFSSLHVRDTKIEVAQFVKDLLL
HLKKLFREGRFNESKYGPPCPPCPAPEFEGGPSVFLFPPKPKDTLMISRT
PEVTCVVVDVSQEDPEVQFNWYVDGVEVHNAKTKPREEQFQSTYRVVSVL
TVLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFFL
YSRLTVDKSRWQEGNVFSCSVMHEALHNHYTQKSLSLSLGKMALIVLGGV
AGLLLFIGLGIFFKRGRKKLLYIFKQPFMRPVQTTQEEDGCSCRFPEEEE
GGCELGGGRVKFSRSADAPAYQQGQNQLYNELNLGRREEYDVLDKRRGRD
PEMGGKPRRKNPQEGLYNELQKDKMAEAYSEIGMKGERRRGKGHDGLYQG
LSTATKDTYDALHMQALPPR

### SEQ ID NO:76

Described herein is a nucleic acid molecule comprising a nucleotide sequence encoding a chimeric antigen receptor (CAR), wherein the chimeric antigen receptor comprises: a targeting domain comprising the amino acid sequence of SEQ ID NO:C; a spacer, a transmembrane domain; a co-stimulatory domain; and a CD3ζ signaling domain. In various embodiments: the transmembrane domain is selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; the wherein the IL13 receptor targeting domain comprises or consists of the amino acid sequence of SEQ ID NO: C with up to 3 single amino acid substitutions (in some cases the Y at position 13 is not substituted); the costimulatory domain is selected from: a 41BB costimulatory domain or variant thereof having 1-5 amino acid modifications, a CD28 costimulatory domain or variant thereof having 1-5 amino acid modifications; a CD28gg costimulatory domain or variant thereof having 1-5 amino acid modifications wherein the costimulatory domain is a 41BB costimulatory domain; the 41BB costimulatory domain comprises the amino acid sequence of SEQ ID NO: 24 or a variant thereof having 1-5 amino acid modifications; the CD3ζ signaling domain comprises the amino acid sequence of SEQ ID NO:21 or a variant thereof comprising any of SEQ ID NOs: 50-56; a linker of 3 to 15 amino acids is located between the 4-1BB costimulatory domain and the CD3ζ signaling domain or variant thereof, the CAR comprises the amino acid sequence of SEQ ID NOs: 70-76 or a variant thereof having 1-5 amino acid modifications; the CAR comprises or consists of an amino acid sequence that is least about 80%, 85%, 90%, 95%, 96%, 97%, 98%, 99%, or 100% identical to any of SEQ ID NOs: 70-76; the CAR comprises an amino acid sequence that has no more than 5, 4, 3, 2, or 1 single amino acid substitutions and or deletions compared to any of SEQ ID NOs: 70-76. Also described is an expression vector comprising any of the forgoing nucleic acid molecules. Also described is a viral vector comprising any of the forgoing nucleic acid molecules.

The CAR can comprise an scFv targeted to any cancer cell antigen, e.g., CD19, MUC16, MUC1, tMUC1, CAIX, CEA, CD20, CD22, CD30, HER-2, MAGEA3, p53, PSCA, BCMA, CD123, CD44V6, Integrin B7, ICAM-1, CD70, CEA, GD2, PSMA, B7H3, CD33, Flt3, CLL1, folate receptor, EGFR, CD7, EGFRvIII, glypican3, CD5, ROR1, CS1, AFP, CD133, and TAG-72. The CAR can comprise a ligand, e.g., an IL-13 or a variant thereof, a chlorotoxin or a variant thereof, etc.

Thus, useful CAR for co-expression include those described in: WO 2016/044811, WO 2017/079694, WO 2017/066481, and WO 2017/062628.

Also described is a population of human T cells, NK cells, myeloid cells, gamma delta T cells, or iPSC-derived effector cells containing any of the forgoing nucleic acid molecules. Also described is a population of human T cells containing any of the forgoing expression vectors or viral vectors. In various embodiments, the population of human T cells comprise central memory T cells, naive memory T cells, pan T cells, or PBMC substantially depleted for CD25+ cells and CD14+ cells.

Also described is a method of treating a patient suffering from a cancer (e.g., brain cancer (glioblastoma), pancreatic, melanoma, neuroblastoma, liver, sarcoma, colorectal, gastric, ovarian carcinoma, fallopian tube, thyroid, bladder, cervical, digestive system, head and neck, osteosarcoma, renal cell carcinoma, prostate cancer, breast cancer or lung cancer), comprising administering a population of autologous or allogeneic human T cells harboring a nucleic acid described herein. In various embodiments, the cells are administered locally or systemically; and are administered by single or repeat dosing.

Also described herein is a method of preparing CAR T cells comprising: providing a population of autologous or allogeneic human T cells and transducing the T cells by a vector comprising a nucleic acid molecule described herein.

Also described are T cells harboring a vector or nucleic acid expressing the CAR and IFNγ. In various embodiments: at least 20%, 30%, or 40% of the transduced human T cells are central memory T cells; at least 30% of the transduced human T cells are CD4+ and CD62L+ or CD8+ and CD62L+. In various embodiments: the population of human T cells comprise a vector expressing a chimeric antigen receptor comprising an amino acid sequence selected from SEQ ID NOs: C or 70-76 or a variant thereof having 1-5 amino acid modifications (e.g., 1 or 2) amino acid modifications (e.g., substitutions); the population of T cells can include one or more of effector T cells, effector memory cells, central memory T cells, stem central memory cells and naive T cells; the population of human T cells comprises central memory T cells (TCM cells) e.g., at least 20%, 30%, 40%, 50% 60%, 70%, 80% of the cells are T CM cells, or the population of T cells comprises a combination of central memory T cells, naive T cells and stem central memory cells (TCM/SCM/N cells) e.g., at least 20%, 30%, 40%, 50% 60%, 70%, 80% of the cells are T CM/SCM/N cells. In some embodiments, the population of T cells includes effector T cells and effector memory cells. In some embodiments, the population of T cells includes both CD4+ cells and CD8+ cells (e.g., at least 20% of the CD3+ T cells are CD4+ and at least 3% of the CD3+ T cells are CD8+ and at least 70, 80 or 90% are either CD4+ or CD8+; at least 15%, 20%, 25%, 30%, 35%, 40%, 50%, 60% of the cells CD3+ cells are CD4+ and at least 4%, 5%, 8%, 10%, 20 of the CD3+ cells are CD8+ cells). In some embodiments, the population of human T cells are autologous to the patient. In some embodiments, the population of human T cells are allogenic to the patient. In some embodiments, T cells expressing a CAR and an IFNγ are called, inter alia, IL13Rα2-IFNγ CAR T cells, IL13Rα2-CAR/IFNγ T cells, and IL13 CAR T-IFNγ cells, interchangeably throughout.

In various embodiments: the spacer domain is selected from the group consisting of: and IgG4(EQ) spacer domain, a IgG4(HL-CH3) spacer domain and an IgG4(CH3) spacer domain; the spacer domain comprises SEQ ID NO: 10; the spacer domain comprises SEQ ID NO: 9; the spacer domain comprises SEQ ID NO: 12; the transmembrane domain is selected from the group consisting of: a CD4 transmembrane domain, a CD8 transmembrane domain, and a CD28 transmembrane domain; the co-stimulatory domain is selected from a CD28 costimulatory domain, and CD28gg costimulatory domain, and a 41-BB co-stimulatory domain.

Also disclosed is a nucleic molecule comprising a nucleotide sequence encoding a chimeric antigen receptor (CAR), wherein the chimeric antigen receptor comprises: targeting domain comprising an amino acid sequence comprising a variant IL13 domain comprising 109, 110, 111, 112, 113 contiguous amino acids of SEQ ID NO: C or the entirety of SEQ ID NO: C with 1, 2, 3, 4 or 5 single amino acid changes, a spacer domain; a transmembrane domain; a costimulatory domain and a CD3, signaling domain.

In various embodiments: the spacer domain comprises the amino acid sequence of any of SEQ ID NOs: 2-12; the costimulatory domain comprises the amino acid sequence of any of SEQ ID NOs: 22-25; and a CD3zeta domain or a variant thereof. In some cases the CAR comprises a CD28 co-stimulatory domain and a variant CD3zeta domain.

Also disclosed is: a vector or an expression vector comprising a nucleic acid molecule described herein; a population of human T cells or NK harboring a nucleic acid molecule described herein.

In various embodiments: the population of human T cells comprise central memory T cells, naive memory T cells, pan T cells, or PBMC substantially depleted for CD25+ cells and CD14+ cells.

Also described is a method of treating a patient suffering from glioblastoma, pancreatic ductal adenocarcinoma, melanoma, ovarian carcinoma, renal cell carcinoma, breast cancer or lung cancer, comprising administering a population of autologous or allogeneic cells harboring a nucleic acid molecule described herein. In various embodiments: the cells are administered locally or systemically or intraventricularly; by single or repeat dosing.

Also described is a method of preparing CAR T cells comprising: providing a population of autologous or allogeneic human T cells or NK and transducing the cells with a vector comprising a nucleic acid molecule described herein.

Also described is a polypeptide encoded by a nucleic acid described herein.

In various embodiments, the NK cells are derived from cord blood, peripheral blood or stem cells.

The CAR or polypeptide can be expressed with additional sequences that are useful for monitoring expression, for example, a T2A or P2A skip sequence and a truncated EGFR or truncated CD19 or LNGFR (can consist of or comprise the amino acid sequence of SEQ ID NO:31).

A non-endogenous or exogenous nucleic acid molecule (or polypeptide) is a nucleic acid molecule (or polypeptide) that is not endogenously present in a cell. The term includes recombinant nucleic acid molecule (or polypeptide) expressed in a cell. An exogenous nucleic acid is a nucleic acid not present in a native wild-type cell; for example, an exogenous nucleic acid may vary from an endogenous counterpart by sequence, by position/location. An exogenous nucleic acid molecule can be introduced into a cell by genetic engineering, either into the cell or a progenitor of the cell. An exogenous nucleic acid molecule encoding a polypeptide can be linked to an expression control sequence and can include a sequence encoding a signal sequence, one or both of which can be heterologous to the sequence encoding the polypeptide.

### Spacer Region

The CAR or polypeptide described herein can include a spacer located between the targeting domain (i.e., IL13 or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge (S→P) | 12 aa | ESKYGPPCPPCP (SEQ ID NO: 3) |
| (S228P) | | |
| IgG4 hinge | 12 aa | ESKYGPPCPSCP (SEQ ID NO: 4) |
| IgG4 hinge (S228P) + linker | 22 aa | ESKYGPPCPPCPGGGSSGGGSG (SEQ ID NO: 5) |
| CD28 hinge | 39 aa | IEVMYPPPYLDNEKSNGTIIHVKGKHLCPSPLFPGPSKP (SEQ ID NO: 6) |
| CD8 hinge-48aa | 48 aa | AKPTTTPAPRPPTPAPTIASQPLSLRPEACRPAAGGAVHTRGLDFACD (SEQ |
| | | ID NO: 7) |
| CD8 hinge-45aa | 45aa | TTTPAPRPPTPAPTIASQPLSLRPEACRPAAGGAVHTRGLDFACD (SEQ ID |
| | | NO: 8) |
| IgG4(HL-CH3) | 129 aa | ESKYGPPCPPCPGGGSSGGGSGGQPREPQVYTLPPSQEEMTKNQVSLTCL |
| Also called IgG4(HL-ΔCH2) | | VKGFYPSDIAVEWESNGQPENNYKTTPPVLDSDGSFFLYSRLTVDKSRWQE |
| (includes 5228P in hinge) | | GNVFSCSVMHEALHNHYTQKSLSLSLGK (SEQ ID NO: 9) |
| IgG4(L235E, N297Q) | 229 aa | ESKYGPPCPSCPAPEFEGGPSVFLFPPKPKDTLMISRTPEVTCVVVDVSQED |
| | | PEVQFNWYVDGVEVHNAKTKPREEQFQSTYRVVSVLTVLHQDWINGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 10) |
| IgG4(S228P, L235E, N297Q) | 229 aa | ESKYGPPCPPCPAPEFEGGPSVFLFPPKPKDTLMISRTPEVTCVVVDVSQED |
| | | PEVQFNWYVDGVEVHNAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: 11) |
| IgG4(CH3) | 107 aa | GQPREPQVYTLPPSQEEMTKNQVSLTCLVKGFYPSDIAVEWESNGQPENN |
| Also called IgG4(ΔCH2) | | YKTTPPVLDSDGSFFLYSRLTVDKSRWQEGNVFSCSVMHEALHNHYTQKSL |
| | | SLSLGK (SEQ ID NO: 12) |
| IgG1 Hinge | 16 aa | LEPKSCDKTHTCPPCP (SEQ ID NO: 44) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain (called CH3 or ΔCH2) or both a CH3 domain and a CH2 domain. The immunoglobulin derived sequences can include one or more amino acid modifications, for example, 1, 2, 3, 4 or 5 substitutions, e.g., substitutions that reduce off-target binding.

The spacer region can also comprise an IgG4 hinge region having the sequence ESKYGPPCPSCP (SEQ ID NO:4) or ESKYGPPCPPCP (SEQ ID NO:3). The spacer region can also comprise the hinge sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGK (SEQ ID NO:12). Thus, the entire spacer region can comprise the sequence:

(SEQ ID NO: 11)
ESKYGPPCPPCPGGGSSGGGSGGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK.

### Transmembrane Domain

A variety of transmembrane domains can be used in the CAR. In some cases, the transmembrane domain is a CD28 transmembrane domain that includes a sequence that is at least 900/%, at least 95%, at least 98% identical to or identical to: FWVLVVVGGVLACYSLLVTVAFIIFWV (SEQ ID NO:14). In some cases, the CD28 transmembrane domain has 1, 2, 3, 4 of 5 amino acid changes (preferably conservative) compared to SEQ ID NO:14. Table 2 includes examples of suitable transmembrane domains. Where a spacer region is present, the transmembrane domain (TM) is located carboxy terminal to the spacer region.

TABLE 2
Examples of Transmembrane Domains

| Name | Accession | Length | Sequence |
|---|---|---|---|
| CD3z | J04132.1 | 21 aa | LCYLLQGILFIYGVILTALFL |
| | | | (SEQ ID NO: 13) |
| CD28 | NM_006139 | 27aa | FWVLVVVGGVLACYSLLVTVAFII |
| | | | FWV |
| | | | (SEQ ID NO: 14) |
| CD28(M) | NM_006139 | 28aa | MFWVLVVVGGVLACYSLLVTVAFI |
| | | | IFWV |
| | | | (SEQ ID NO: 15) |
| CD4 | M35160 | 22aa | MALIVLGGVAGLLLFIGLGIFF |
| | | | (SEQ ID NO: 16) |
| CD8tm | NM_001768 | 21aa | IYIWAPLAGTCGVLLLSLVIT |
| | | | (SEQ ID NO: 17) |
| CD8tm2 | NM_001768 | 23aa | IYIWAPLAGTCGVLLLSLVITLY |
| | | | (SEQ ID NO: 18) |
| CD8tm3 | NM_001768 | 24aa | IYIWAPLAGTCGVLLLSLVITLYC |
| | | | (SEQ ID NO: 19) |
| 41BB | NM_001561 | 27aa | IISFFLALTSTALLFLLFFLTLRF |
| | | | SVV |
| | | | (SEQ ID NO: 20) |
| NKG2D | NM_007360 | 21aa | PFFFCCFIAVAMGIRFIIMVA |
| | | | (SEQ ID NO: 65) |

### Costimulatory Domain

The costimulatory domain can be any domain that is suitable for use with a CD3ζ signaling domain. In some cases, the co-signaling domain is a CD28 co-signaling domain that includes a sequence that is at least 90%, at least 95%, at least 98% identical to or identical to: RSKRSRLLHSDYMNMTPRRPGPTRKHYQPYAPPRDFAAYRS (SEQ ID NO: 22). In some cases, the 4-1BB co-signaling domain has 1, 2, 3, 4 of 5 amino acid changes (preferably conservative) compared to SEQ ID NO:22.

The costimulatory domain(s) are located between the transmembrane domain and the CD3ζ signaling domain. Table 3 includes examples of suitable costimulatory domains together with the sequence of the CD3ζ signaling domain.

TABLE 3
CD3 Domain and Examples of Costimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR |
| | | | (SEQ ID NO: 21) |
| | | | ITAMS 1-3 underlined |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLFQGLSTATKDTFDALHMQALPPR |
| | | | (SEQ ID NO: 50) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLYQGLSTATKDTYDALHMQALPPR |
| | | | (SEQ ID NO: 51) |
| CD3ζ | | 113 aa | RVKFSRSADAPAYQQGQNQLYNELNLGRREEYDVLDKRRGR |
| variant | | | DPEMGGKPRRKNPQEGLYNELQKDKMAEAYSEIGMKGERR |
| | | | RGKGHDGLFQGLSTATKDTFDALHMQALPPR |
| | | | (SEQ ID NO: 52) |
| CD3ζ | | 113 aa | RVKFSRSADAPAYQQGQNQLYNELNLGRREEYDVLDKRRGR |
| variant | | | DPEMGGKPRRKNPQEGLYNELQKDKMAEAFSEIGMKGERR |
| | | | RGKGHDGLFQGLSTATKDTFDALHMQALPPR |
| | | | (SEQ ID NO: 53) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAYSEIGMKGERR |
| | | | RGKGHDGLFQGLSTATKDTFDALHMQALPPR |
| | | | (SEQ ID NO: 54) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLYQGLSTATKDTFDALHMQALPPR |
| | | | (SEQ ID NO: 55) |
| CD3ζ | | 113 aa | RVKFSRSADAPAYQQGQNQLYNELNLGRREEYDVLDKRRGR |
| variant | | | DPEMGGKPRRKNPQEGLFNELQKDKMAEAFSEIGMKGERR |
| | | | RGKGHDGLFQGLSTATKDTYDALHMQALPPR |
| | | | (SEQ ID NO: 56) |
| CD28 | NM_006139 | 42aa | RSKRSRLLHSDYMNMTPRRPGPTRKHYQPYAPPRDFAAYRS |
| | | | (SEQ ID NO: 22) |
| CD28gg* | NM_006139 | 42aa | RSKRSRGGHSDYMNMTPRRPGPTRKHYQPYAPPRDFAAYRS |
| | | | (SEQ ID NO: 23) |
| 41BB | NM_001561 | 42 aa | KRGRKKLLYIFKQPFMRPVQTTQEEDGCSCRFPEEEEGGCEL |
| | | | (SEQ ID NO: 24) |
| OX40 | NM_003327 | 42 aa | ALYLLRRDQRLPPDAHKPPGGGSFRTPIQEEQADAHSTLAKI |
| | | | (SEQ ID NO: 25) |
| 2B4 | NM_016382 | 120 aa | WRRKRKEKQSETSPKEFLTIYEDVKDLKTRRNHEQEQTFPGG |
| | | | GSTIYSMIQSQSSAPTSQEPAYTLYSLIQPSRKSGSRKRNHSPS |
| | | | FNSTIYEVIGKSQPKAQNPARLSRKELENFDVYS |
| | | | (SEQ ID NO: 66) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and a short linker consisting of 2-10, e.g., 3 amino acids (e.g., GGG) is can be positioned between the costimulatory domain and the CD3ζ signaling domain.

### CD3ζ Signaling Domain

The CD3′ signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO:21). In some cases, the CD3ζ signaling domain has 1, 2, 3, 4 of 5 amino acid changes (preferably conservative) compared to SEQ ID NO:21. In some case the CD3ζ signaling domain comprises any of SEQ ID NOs: 50-56. These variant CD3ζ signaling domains have Y to F mutations in one or more ITAM domains. In some cases it is preferable to use a variant with mutations that inactive ITAMs 2 and 3.

### Human IFNγ

The IFNγ domain is a domain that includes at least a functional portion of mature human IFN/(e.g., amino acids 24-161 human IFNγ; GenBank NP_000610) or a functional portion of mature human IFNγ.

Mature human IFNγ has the sequence: QDPYVKE AENLKKYFNA GHSDVADNGT LFLGILKNWKEESDRKIMQS QIVSFYFKLF KNFKDDQSIQ KSVETIKEDM NVKFFNSNKK KRDDFEKLTNYSVTDLNVQR KAIHELIQVM AELSPAAKTG KRKRSQMLFR GRRASQ (mature IFNγ; SEQ ID NO.1). Immature human IFNγ (includes a signal sequence) has the sequence: MKYTSYILAF QLCIVLGSLG CYCQDPYVKE AENLKKYFNA GHSDVADNGT LFLGILKNWK EESDRKIMQS QIVSFYFKLF KNFKDDQSIQ KSVETIKEDM NVKFFNSNKK KRDDFEKLTN YSVTDLNVQR KAIHELIQVM AELSPAAKTG KRKRSQMLFR GRRASQ (SEQ ID NO: B). In some embodiments, a human IFNγ comprises the sequence:

(SEQ ID NO: Z)
QDPYVKEAENLKKYFNAGHSDVADNGTLFLGILKNWKEESDRKIMQSQIV
SFYFKLFKNFKDDQSIQKSVETIKEDMNVKFFNSNKKKRDDFEKLTNYSV
TDLNVQRKAIHELIQVMAELSPAAKTGKRKRSQ

In some cases, the IFNγ domain has 1, 2, 3, 4 or 5 amino acid changes (preferably conservative) compared to SEQ ID NO:1 or SEQ ID NO:B or SEQ ID NO: Z. For example, 1, 2 or all 3 of the following amino acid changes can be made in SEQ ID NO: 1 or SEQ ID NO:Z: K74A, E75Y and N83R. In some embodiments, an IFNγ domain provided herein comprise an amino acid sequence having at least 95% identity to SEQ ID NO: 1 or SEQ ID NO:B or SEQ ID NO: Z. In some embodiments, an IFNγ comprises at least one amino acid substitution at a position corresponding to an amino acid residue selected from Q1, D2, P3, K6, Q64, Q67, K68, E71, T72, K74, E75, D76, N78, V79, K80, N83, S84, K86, R89, D90, and any combination thereof of SEQ ID NO: 1 or SEQ ID NO:Z.

In some embodiments, an IFNγ domain provided herein comprise an amino acid sequence having at least 95% identity to SEQ ID NO: 1 or SEQ ID NO: Z, and further including at least one amino acid substitution at a position corresponding to an amino acid residue selected from Q1, D2, P3, K6, Q64, Q67, K68, E71, T72, K74, E75, D76, N78, V79, K80, N83, S84, K86, R89, D90, and any combination thereof of SEQ ID NO: 1 or SEQ ID NO:Z.

In some embodiments, a variant of IFNγ can also be used. A number of IFNγ variants are known in the art and can be useful (Mendoza J L, et. al., (2019) Nature 567:56; WO 2020/028275).

### Truncated EGFR or Truncated CD19 or LNGFR

In some embodiments, a CAR or peptide described herein can comprise a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLHILPVAFR GDSFTHTPPLDPQELDILKTVKEITGFLLIQAWPENRTDLHAFENLEIIRGRTKQHGQFSL AVVSLNITSLGLRSLKEISDGDVIISGNKNLCYANTINWKKLFGTSGQKTKIISNRGENSC KATGQVCHALCSPEGCWGPEPRDCVSCRNVSRGRECVDKCNLLEGEPREFVENSECIQC HPECLPQAMNITCTGRGPDNCIQCAHYIDGPHCVKTCPAGVMGENNTLVWKYADAGH VCHLCHPNCTYGCTGPGLEGCPTNGPKIPSIATGMVGALLLLLVVALGIGLFM (SEQ ID NO:28). In some cases, the truncated EGFR has 1, 2, 3, 4 of 5 amino acid changes (preferably conservative) compared to SEQ ID NO:28.

In some embodiments, a CAR or peptide described herein can comprise a ribosomal skip sequence (e.g., LEGGGEGRGSLLTCGDVEENPGPR; SEQ ID NO:27) and a truncated CD19R (also called CD19t) having a sequence that is at least 90%, at least 95%, at least 98% identical to or identical to: MPPPRLLFFLLFLTPMEVRPEEPLVVKVEEGDNAVLQCLKGTSDGPTQQLTWSRESPLKP FLKLSLGLPGLGIHMRPLAIWLFIFNVSQQMGGFYLCQPGPPSEKAWQPGWTVNVEGSG ELFRWNVSDLGGLGCGLKNRSSEGPSSPSGKLMSPKLYVWAKDRPEIWEGEPPCVPPRD SLNQSLSQDLTMAPGSTLWLSCGVPPDSVSRGPLSWTHVHPKGPKSLLSLELKDDRPAR DMWVMETGLLLPRATAQDAGKYYCHRGNLTMSFHLEITARPVLWHWLLRTGGWKVS AVTLAYLIFCLCSLVGILHLQRALVLRRKR (SEQ ID NO:26). In some cases, the truncated CD19t has 1, 2, 3, 4 of 5 amino acid changes (preferably conservative) compared to SEQ ID NO:26.

In some embodiments, a CAR or peptide described herein can comprise a ribosomal skip sequence (e.g., LEGGGEGRGSLLTCGDVEENPGPR: SEQ ID NO:27) and tEGFR having a sequence that is at least 90%, at least 95%, at least 98% identical to or identical to:

(SEQ ID NO: 45)
MLLLVTSLLLCELPHPAFLLIPRKVCNGIGIGEFKDSLSINATNIKHFKN
CTSISGDLHILPVAFRGDSFTHTPPLDPQELDILKTVKEITGFLLIQAWP
ENRTDLHAFENLEIIRGRTKQHGQFSLAVVSLNITSLGLRSLKEISDGDV
IISGNKNLCYANTINWKKLFGTSGQKTKIISNRGENSCKATGQVCHALCS
PEGCWGPEPRDCVSCRNVSRGRECVDKCNLLEGEPREFVENSECIQCHPE
CLPQAMNITCTGRGPDNCIQCAHYIDGPHCVKTCPAGVMGENNTLVWKYA
DAGHVCHLCHPNCTYGCTGPGLEGCPTNGPKIPSIATGMVGALLLLLVVA
LGIGLFM.

In some embodiments, a CAR or peptide described herein can comprise a ribosomal skip sequence and a truncated LNGFR having a sequence that is at least 90%, at least 95%, at least 98% identical to or identical to: MGAGATGRAMDGPRLLLLLLLGVSLGGAKEACPTGLYTHSGECCKACNLGEGVAQPC GANQTVCEPCLDSVTFSDVVSATEPCKPCTECVGLQSMSAPCVEADDAVCRCAYGYYQ DETTGRCEACRVCEAGSGLVFSCQDKQNTVCEECPDGTYSDEANHVDPCLPCTVCEDT ERQLRECTRWADAECEEIPGRWITRSTPPEGSDSTAPSTQEPEAPPEQDLIASTVAGVVTT VMGSSQPVVTRGTTDNLIPVYCSILAAVVVGLVAYTAFKRWNSCKQNK (SEQ ID NO:CC). In some cases, the truncated LNGFR has 1, 2, 3, 4 of 5 amino acid changes (preferably conservative) compared to SEQ ID NO:CC.

Other ribosomal skip sequences useful in a CAR or peptide described herein include T2At having a sequence that is at least 95% identical to: EGRGSLLTCGDVEENPGP (SEQ ID NO:46) or P2A having a sequence that is at least 95% identical to: GSGATNFSLLKQAGDVEENPGP (SEQ ID NO:47). In some cases, the ribosomal skip sequence has 1, 2, 3, 4 of 5 amino acid changes (preferably conservative) compared to SEQ ID NO:46 or 47.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

In some cases, the CAR can be produced using a vector in which the CAR open reading frame is followed by a ribosome skip sequence and a truncated EGFR (EGFRt), which lacks the cytoplasmic signaling tail, or a truncated CD19R or a LNGFR. In this arrangement, co-expression of EGFRt provides an inert, non-immunogenic surface marker that allows for accurate measurement of gene modified cells, and enables positive selection of gene-modified cells, as well as efficient cell tracking of the therapeutic NK cells in vivo following adoptive transfer. Efficiently controlling proliferation to avoid cytokine storm and off-target toxicity is an important hurdle for the success of NK cell immunotherapy. The EGFRt, CD19t, or LNGFR incorporated in the CAR lentiviral or retroviral vector can act as suicide gene to ablate the CAR+ cells in cases of treatment-related toxicity.

In some cases, a nucleic acid molecule described herein comprises a promoter that controls expression of both the CAR and human interferon gamma. In other cases, a nucleic acid molecule described herein comprises a first promoter controls expression of the CAR and a second promoter controls expression of human interferon gamma. In some cases, the first and second promoters are identical and in some cases they are different. In some embodiments, the first promoter is a strong constitutive promoter or an inducible promoter. In some embodiments, the second promoter is a weaker promoter than the first promoter or is an inducible promoter. Useful promoters are well-known in the art. For example, synthetic NFAT promoter can be used in a nucleic acid encoding a CAR construct. Useful promoters can comprise one or more of CMV, EF1, SV40, PKG1, PKG100, Ubc, Tetracycline, Doxycycline, NFAT, and any other constitutive or inducible promoter. In some embodiments, a NFAT recognition element can be used (TGGAGGAAAAACTGTTTCATACAGAAGGCG; SEQ ID NO: X). In some embodiments, a useful promoter comprises one, two, three, four, five, six, seven, eight, nine, ten, or eleven repeats of the NFAT recognition element. In some embodiments, a useful promoter comprises any one or more of SEQ ID NO: X, X2G X3, X4, X5, X6, X7, X8, X9, X10 and X11.

SEQ ID NO: X2
TGGAGGAAAAACTGTTTCATACAGAAGGCGTGGAGGAAAAACTGTTTCAT
ACAGAAGGCG;
SEQ ID NO: X3
TGGAGGAAAAACTGTTTCATACAGAAGGCGTGGAGGAAAAACTGTTTCAT
ACAGAAGGCGTGGAGGAAAAACTGTTTCATACAGAAGGCG;
SEQ ID NO: X4
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCG;
SEQ ID NO: X5
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGC
G;
SEQ ID NO: X6
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCG;
SEQ ID NO: X7
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCGTGGAGGAAAAACTGTTTCAT
ACAGAAGGCG;
SEQ ID NO: X8
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCGTGGAGGAAAAACTGTTTCAT
ACAGAAGGCGTGGAGGAAAAACTGTTTCATACAGAAGGCG;
SEQ ID NO: X9
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCGTGGAGGAAAAACTGTTTCAT
ACAGAAGGCGTGGAGGAAAAACTGTTTCATACAGAAGGOGTGGAGGAAAA
ACTGTTTCATACAGAAGGCG;
SEQ ID NO: X10
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGC
G;
SEQ ID NO: X11
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCGTGGAGGAAAAACTGTTTCAT
ACAGAAGGCGTGGAGGAAAAACTGTTTCATACAGAAGGCGTGGAGGAAAA
ACTGTTTCATACAGAAGGCGTGGAGGAAAAACTGTTTCATACAGAAGGCG
TGGAGGAAAAACTGTTTCATACAGAAGGCG;

The CAR or polypeptide described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably an immune cell (e.g., a T cell), and most preferably an autologous T cell.

The CAR or polypeptide can be transiently expressed in a cell population by an mRNA encoding the CAR or polypeptide. The mRNA can be introduced into the immune cells by electroporation (Wiesinger et al. 2019 Cancers (Basel) 11:1198).

In some embodiments, described herein is a method of increasing survival of a subject having cancer comprising administering a composition comprising a CAR immune cell described herein.

In some embodiments, described herein is a method of treating a cancer in a patient comprising administering a composition comprising a CAR immune cell described herein.

In some embodiments, described herein is a method of reducing or ameliorating a symptom associated with a cancer in a patient comprising administering a composition comprising a CAR immune cell described herein.

In some embodiments, a composition comprising CAR NK cells or CAR T cells described herein is administered locally or systemically. In some embodiments, a composition comprising CAR immune cells described herein is administered by single or repeat dosing. In some embodiments, a composition comprising CAR immune cells described herein is administered to a patient having a cancer, a pathogen infection, an autoimmune disorder, or undergoing allogeneic transplant.

In some embodiments, the cancer is a solid tumor. In some embodiments, the cancer is melanoma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety for any and all purposes.

Other features and advantages of the described compositions and methods will be apparent from the following detailed description and figures, and from the claims.

### DESCRIPTION OF DRAWINGS

FIGS. 1A-1D: mTL13BBζ generation and phenotypic characterization of mIL13BBζ CAR T cells. 1A, Schematic depicting the murine IL13Rα2-CAR T (mIL13BBζ CAR T) construct. 1B and 1C, Flow cytometry and graph depicting phenotypic changes of murine CAR T cell from day 0 to 4. 1D, In vitro killing of mIL13BBζ CAR T cells against IL13Rα2+ and IL13Rα2-K-Luc cells.

FIGS. 2A-2I: Murine IL13BBζ CAR T cells have potent antitumor activity. 2A, Flow cytometry (left panel) and bar graph summarizing percent CAR+ T cells demonstrating transduction efficiency (right). 2B and 2C, Immunofluorescent and flow cytometry staining confirmed transduction of mIL13Rα2 in KR158 (K-Luc) glioma cells. 2D, In vitro killing of mIL13BBζ CAR T cells against K-Luc cells (E:T, 1:3). 2E, Luminex detected IFNγ and TNFα levels. 2F, Schematic depicting in vivo experimental design. 2G, Images of hematoxylin and eosin (H&E) showed invasive K-Luc in untreated and CAR T treated brains. 2H, Survival curve of mice bearing K-Luc-mIL13Rα2+ tumors in untreated and CAR T-treated groups. 21, Bioluminescent images (BLI; top) and flux values (bottom) show tumor growth in untreated and CAR T-treated groups. Data of at least three independent experiments were presented as means±s.e.m. (2D and 2E) and were analyzed by two-tailed, unpaired Student's t-test. Differences between survival curves were analyzed by log-rank (Mantel-Cox) test (2H). *p<0.05, **p<0.01, and ***p<0.001 for indicated comparison.

FIGS. 3A-3F: mIL13BBζ CAR T cells have potent antitumor activity and induced endogenous memory immune response against GL261-Luc tumors. 3A, Images of hematoxylin and eosin (H&E) showed morphology of GL261-Luc tumor. 3B, Immunofluorescent and flow cytometry staining confirmed transduction of IL13Rα2 in GL261-Luc glioma cells. 3C, In vitro killing of mIL13BBζ CAR T cells against IL13Rα2+GL261-Luc glioma cells (E:T, 1:3). 3D, Luminex ELISA detected IFNγ and TNFα levels. 3E, Survival curve of mice bearing IL13Rα2+GL261-Luc glioma tumors in untreated and CAR T-treated groups. 3F, Survival of mice cured by the CAR T therapy and rechallenged with IL13Rα2 negative GL261-Luc. Data of at least two independent experiments were presented as means t s.e.m. (3C) and were analyzed by two-tailed, unpaired Student's 1-test. *p<0.05, **p<0.01, ***p<0.001 and ****p<0.0001 for indicated comparison. Differences between survival curves (3E) were analyzed by log-rank (Mantel-Cox) tested by log-rank (Mantel-Cox) test.

FIGS. 4A-4E: mIL13BBζ CAR T cells have a superior antitumor activity in immunocompetent host. 4A, Schema depicting in vivo experimental design with CAR administration at day 4 or 7. 4B, Bioluminescent images (BLI; top) and flux values (bottom) showed tumor growth in untreated and CAR T-treated group in 4-day old tumor model. 4C, Survival curve of mice bearing 4-day old K-Luc IL13Rα2+ tumors in untreated and CAR T-treated groups. 4D, Bioluminescent images (BLI; top) and flux values (bottom) showed tumor growth in untreated and CAR T-treated group in 7-day old tumor model. 4E, Survival curve of mice bearing 7-day old K-Luc IL13Rα2+ tumors in untreated and CAR T-treated groups. Differences between survival curves (4C, 4E) were analyzed by log-rank (Mantel-Cox) test.

FIG. 5a-5f: CAR T cells induce endogenous memory immune response and generation of antigen specific T cells. a, Survival of mice cured by CAR T therapy and rechallenged with IL13Rα2 negative K-Luc tumors. b, Bioluminescent (BLI) images (top) and flux values (bottom) show tumor growth in naive controls and survivors of CAR T therapy groups. c, Overview of experimental design. d, In vitro killing and e, expansion of endogenous T cells isolated from untreated or CAR T-treated mice against K-Luc-mIL13Rα2+ cells (E:T, 10:1). f, Assessment of in vivo killing capacity of isolated CAR T cells and endogenous T cells from untreated or CAR T-treated cells in tumor (K-Luc) bearing mice. Data are inclusive of at least two independent experiments. Each symbol represents one individual. Data are presented as means±s.e.m. (d and e) and were analyzed by two-tailed, unpaired Student's t-test. Differences between survival curves were analyzed by log-rank (Mantel-Cox) test (a). *p<0.05, **p<0.01, and ***p<0.001 for indicated comparison.

FIG. 6a-6b: mIL13BBζ CAR T cells have a potent antitumor activity but are unable to induce endogenous memory immune response in small tumor model. a, Survival curve of mice bearing 4 day-old K-Luc IL13Rα2+ tumors in untreated and CAR T-treated groups. b, Survival of mice cured by the CAR T therapy and rechallenged with IL13Rα2 negative K-Luc tumors. Differences between survival curves (a) were analyzed by log-rank (Mantel-Cox) test.

FIG. 7a-7e: Comparison of survival in mice bearing mixed antigen tumors. a, Schema of day 4 and 7 in vivo experimental design. b, Flow cytometry showing different levels of IL13Rα2. c, Survival curve of mice bearing day 4 K-Luc IL13Rα2+ tumors in untreated and CAR T-treated groups. d, Survival curve of mice bearing day 7 K-Luc IL13Rα2+ tumors in untreated and CAR T-treated groups. e, Quantification of CD11b and CD3 cells after flow cytometry sort of untreated, K-Luc tumor bearing mice. Each symbol represents one individual. Data are presented as means±s.e.m. (e) and were analyzed by two-tailed, unpaired Student's t-test. *p<0.05, **p<0.01, ***p<0.001 and ****p<0.0001 for indicated comparison. Differences between survival curves (c, d) were analyzed by log-rank (Mantel-Cox) test.

FIG. 8: Flow Cytometry sort of Endogenous and CAR T cells. A representation of flow cytometry sort of endogenous (CD3+CD19−) and CAR T (CD3+CD19+) populations.

FIG. 9a-9g: CAR T cells activate endogenous T cells in glioma tumor microenvironment. a, Nanostring analysis shows global changes in gene expression of intratumoral T cells (CD3+) isolated from untreated or CAR T-treated mice 3 days post-therapy. b, UMAP plots depict changes in lymphoid compartments in the glioma TME after CAR T therapy. c, Feature plots demonstrate phenotypic characterization of T cell subclusters and enriched pathways within CD8 and CD4 T cell subclusters post therapy. d, Heatmap of enrichment scores (GSEA analysis) shows enriched pathways in T cell subclusters. e, Experimental design demonstrating the adoptive transfer of CD45.1+ mock or CAR T cells (top) and flow cytometry analysis show frequency of endogenous (CD3+CD45.2+) or adoptively transferred T cells (CD3+CD45.1+) in glioma TME (bottom). f, Bar graphs compare adoptively transferred mock (CD3+CD45.1+) or CAR T cell (CD3+CD45.1+CD19+) number and phenotypic characterization (CD69, Ki67 and GZMB). g, Bar graphs compare endogenous T cell (CD3+CD45.2+) numbers and phenotypic characterization (CD69, Ki67 GZMB, and IFNγ) in untreated, mock or CAR T treated mice (n=5 per group). Data are inclusive of at least two independent experiments. Data are presented as means±s.e.m. (F and G) and were analyzed by two-tailed, unpaired Student's t-test. *p<0.05, **p<0.01, and ***p<0.001 for indicated comparison.

FIG. 10a-10d: Single cell RNA sequencing of intratumoral immune cells. a, UMAP plot from merged untreated and CAR T-treated data of exclusively intratumoral immune cells. b, Feature plot of immune subset-specific marker-gene expression. c, Changes in frequency of lymphoid subclusters (top) and violin plots (bottom) depict lymphoid specific markers. d, Bar graph demonstrates changes in frequency of myeloid subclusters (top) and violin plots (bottom) depict myeloid specific markers.

FIG. 11a-11b: Single-cell RNA sequencing identifies phenotypes of intratumoral T cells. a, The UMAP plots demonstrate enhancement of genes associated with memory stem like T cells after CAR T therapy. b, The UMAP plots demonstrate reduction in expression of genes associated with T regulatory cells after CAR T therapy.

FIG. 12: Expression of genes associated with T cell activation in intratumoral T cells. qPCR analysis shows genes associated with T cell activation. Data are presented as means t s.e.m. and were analyzed by two-tailed, unpaired Student's t-test. *p<0.05, **p<0.01, ***p<0.001 and ****p<0.0001 for indicated comparison.

FIG. 13a-13f: CAR T cells activate the resident myeloid population in glioma tumor microenvironment. a, UMAPs depict changes in intratumoral myeloid cells from CAR T-treated or untreated mice. b, Enrichment plot of IFNγ signaling pathways in intratumoral macrophage and microglia cells in CAR T-treated compared with untreated, as identified by the GSEA computational method. c, GSEA analysis reveals upregulation of population specific activation pathways in myeloid subclusters (MP: macrophage; MG: microglia; DC: dendritic cells; Neu: neutrophils). d, Nanostring analysis show global changes in gene expression of myeloid cells (CD11b+) isolated from CAR T-treated vs untreated mice. e, UMAPs indicate relative expression levels of antigen presentation gene signatures at a single-cell level within the myeloid compartment. f, Histograms (left) and bar graphs (right) show intratumoral CD11b+CD45.2+ cells expressing MHCII, MHCI, CD86, and IFNγ. Data are presented as means±s.e.m. (f) and were analyzed by two-tailed, unpaired Student's t-test. *p<0.05, **p<0.01, ***p<0.001, ****p<0.0001 for indicated comparison.

FIG. 14a-14j: Lack of IFNγ in CAR T cells impairs antitumor activity and activation of host immune cells. a, Schematic of the experimental design. b, Comparison of percent CAR positivity, viability, expansion, and CD4:CD8 ratio in CAR Twt and CAR TIFNγ−/−. c, In vitro killing of CAR Twt and CAR TIFNγ−/− against K-Luc-IL13Rα2+ cells (E:T, 1:1). d, A flow cytometry depicts intracellular cytokine levels (TNFα, GZMB and IFNγ) in wt and IFNγ−/− CAR T cells. e, Bioluminescent (BLI) images (top) and flux values (bottom) show tumor growth in untreated, CAR Twt or CAR TIFNγ−/−. f, Survival curve of mice bearing K-Luc-IL13Rα2+ tumors in untreated, CAR Twt and CAR TIFNγ−/− groups. g, Heatmap indicates normalized expression of genes associated with immune activation and suppression in the tumor. h, Bar graphs (left) and flow cytometry plots (left) comparing CAR T cell (CD3+CD19+) number and activation phenotype (CD69). i, Bar graphs (left) and flow cytometry plots (right) comparing endogenous T cell (CD3+CD19) number and activation phenotype (CD69). j, Histograms (left) and bar graphs (right) showing phenotype in myeloid (CD11b+) compartment. Data are inclusive of at least two independent experiments. Each symbol represents one individual. Data are presented as means±s.e.m. (h, i, and j) and were analyzed by two-tailed, unpaired Student's t-test. Differences between survival curves (f) were analyzed by log-rank (Mantel-Cox) test. *p<0.05, **p<0.01, and ***p<0.001 for indicated comparison.

FIG. 15A-15J: Lack of IFNγ production by CAR T cells impairs antitumor activity and activation of host immune cells. A, Schematic of the experimental design. B, Comparison of percent CAR positivity, viability, expansion, and CD4:CD8 ratio in CAR T^{wt} and CAR T^{IFNγ-/-}. C, In vitro killing of CAR T^{wt} and CAR T^{IFNγ-/-} against K-Luc-mIL13Rα2+ cells (E:T, 1:1). D, Representative flow cytometry plot depicts intracellular cytokine levels (TNFα, GZMB and IFNγ) in wt and IFNγ−/− CAR T cells after exposure to K-Luc-mIL13Rα2+ tumors. E, Representative bioluminescent (BLI) images (top) and flux values (bottom) show tumor growth in untreated, CAR T^{wt} or CAR T^{IFNγ-/-}. Individual mice are represented with dotted lines and median flux is shown in thick line. F, Survival curve of mice bearing K-Luc-mIL13Rα2+ tumors in untreated, CAR T^{wt} treated and CAR T^{IFNγ-/-} treated groups. G, Heatmap indicates normalized expression of genes associated with immune activation and suppression in the TME. H, Bar graphs (left) and representative flow cytometry plots (right) comparing CAR T cell (CD3+CD19+) number and activation phenotype (CD69). I. Bar graphs (left) and representative flow cytometry plots (right) comparing endogenous T cell (CD3+CD19−) number and activation phenotype (CD69). J, Representative histograms (left) and bar graphs (right) showing phenotype in myeloid (CD11b+) compartment. Data are representative of at least two independent experiments. Each symbol represents one individual (H, I, and J). Data are presented as means±s.e.m and were analyzed by two-tailed, unpaired Student's t-test. Differences between survival curves (F) were analyzed by log-rank (Mantel-Cox) test. *p<0.05, **p<0.01, and ***p<0.001 for indicated comparison.

FIG. 16a-16d: CAR T cells promote monocyte differentiation and generation of M1 type macrophages. a, Schema of experimental design. b, Flow cytometry (left) and bar graphs (right) depict phenotypic changes in monocytes after incubation with different conditioned media. c, Microscopy images demonstrate morphological change in monocytes after incubation with different conditioned media. d, qPCR analysis of genes associated with M1 macrophage phenotype. Data are presented as means±s.e.m. (d) and were analyzed by two-tailed, unpaired Student's t-test. *p<0.05, **p<0.01, ***p<0.001 and ****p<0.0001 for indicated comparison.

FIG. 17a-17k: CAR T cells can activate GBM patient immune cells. a, Schema of experimental design. b, Flow cytometry, c, microscopy images and d, bar graph summary of phenotypic changes of patient macrophages after incubation in conditioned media. e, Flow cytometry and f, summary of phenotypic changes in patient T cells after incubation in conditioned media. g, Schematic of trial design for patients receiving CAR T therapy. h, Flow cytometry demonstrates intracellular IFNγ levels in patient T cells obtained before CAR T therapy (Pre-CAR), and during response to CAR T therapy (Post-CAR) after coculture with irradiated autologous tumor followed by 4 hour stimulation. i, T cell count after incubation with autologous irradiated (Irr.) patient tumor. j, In vitro killing by T cells against autologous (UPN109) or nonspecific tumor line (K562) at 10:1, E:T ratio. k, Flow cytometry demonstrates the IL13Rα.2 expression of the patient autologous (UPN109) tumor. Each symbol represents one replicate. Data are presented as means±s.e.m. (d, f, i and j) and were analyzed by two-tailed, unpaired Student's t-test. *p<0.05, **p<0.01, ***p<0.001 and ****p<0.0001 for indicated comparison.

FIG. 18: List of primers used in the studies described herein.

FIG. 19: List of antibodies used in the studies described herein.

FIGS. 20A-20D: Molecular design and generation of different IL13Rα2-IFNγ CAR T cell constructs and depicts aspects of the preparation of CAR T cell co-expressing interferon gamma. 20A. Construct design for IL13 CAR T co-expressing IFNγ compared to standard IL13 CAR T. 20B. Schema of transduction and IL13 CAR T cell production. T cells are isolated and activated in the presence of CD3/CD28 antibodies (1:1), followed by IL13 CAR transduction. 2C. Flow cytometry showed the percentage CAR positive cells using IL13 as a marker of CAR expression. 20D. IFNγ levels in various IL13 CAR T cells during production (left panel) and ELISA confirmed IFNγ expression and secretion in CAR T cells confirming transduction and expression of IFNγ within the construct.

FIGS. 21A-21E: Depicts the results of functional and phenotypic assessment of murine IL13 CAR T cells and IL13Rα2-IFNγ CAR T cells. Murine IL13 CAR T cells or IL13 CAR T cells co-expressing IFNγ were co-cultured with murine glioma tumors at 1 effector:3 target ratio. 21A. Microscopy images demonstrated killing capacity of different IL13 CAR T constructs (untransduced, mock T cells, IL13 CAR T only, and 113 CAR IFNγ T cells). 21B. T cell count after 24 hours of co-culture. 21C. Tumor cell count after 24 hours of co-culture. 21D. Bar graph showing percent T cell activation as measured by CD69 expression after 24 hours of co-culture. 21E. Flow cytometry analysis demonstrated comparable exhaustion (PD-1+ Tim3+) and differentiation (CD62L+CD45RA+) phenotype in murine IL13Rα2-IFNγ CAR T cells and IL13Rα2-IFNγ CAR T cells.

FIGS. 22A-22C: Functional and phenotypic assessment of human IL13 CAR cells and IL13Rα2-IFNγ CAR T cell. Human IL13 CAR T or IL13 CAR T-IFNγ cells were co-cultured with patient-derived glioma tumors (1 effector:25 target). 22A. T cell count after 24 hours of co-culture. 22B. Tumor cell count measured after 24 hours of co-culture. 22C. Flow cytometry analysis demonstrated comparable exhaustion (PD-1+ Tim3+) and differentiation (CD62L+CD45RA+) phenotype in human IL13Rα2-IFNγ CAR T cells and IL13Rα2-IFNγ CAR T cells.

FIG. 23: IL13Rα2-IFNγ CAR T cells induced abscopal effect in multi-lesion metastatic melanoma model. Tumors were injected in both flanks. Once tumors reached a predetermined size, IL13Rα2 CAR T cells or TL13Rα2-IFNγ CAR T cells were administered locally to one tumor. Both tumors were measured for antitumor activity. The line graph demonstrated tumor volume changes in the tumor opposite the side of treatment.

FIG. 24: Amino acid sequence of various human 1L13 CAR (SEQ ID NO: 70-72) with the various domains indicated.

FIG. 25: Amino acid sequences of various human IL13 CAR (SEQ ID NO: 73-75) with the various domains indicted.

FIGS. 26A-26B: IL13Rα2-IFNγ CAR T cell can reprogram the macrophages. 26A. Schematic depiction of transduction and CAR T cell production. 26B. Bar graphs demonstrated reprogramming of macrophages with IL13Rα2-IFNγ CAR T cells. Bar graphs showed qPCR analysis of genes associated with proinflammatory and metabolically active macrophages when incubated with supernatant collected during manufacturing of the CAR products or exposed to exogenous IFNγ. Each data point is indicative of one replicate.

FIGS. 27A-27C: Development of the inducible IL13Rα2-IFNγ CAR T cells through a synthetic NFAT promoter. 27A. A schematic depicting the molecular design of the inducible IFNγ expression upon CAR T activation. 27B. An illustration depicting experimental design, briefly, cells were transduced with an NFAT-eGFP-CAR T cell construct and co-cultured with IL13Rα2+ and IL13Rα2− tumors. Upon stimulation with antigen positive tumors and activation, GFP is expressed and detectable. 27C. Flow cytometry demonstrated expression of GFP in cells transduced and activated by antigen positive tumors.

FIGS. 28A-28C: IL13Rα2-IFNγ CAR T cells were more efficacious compared to the standard IL13Rα2 CAR T cells in targeting medium/low IL13Rα2 antigen expressing tumors in vivo. 28A. A schematic depicting the experimental design. 28B. Bar graph showed tumor progression in mice bearing high-IL13Rα2 antigen expressing tumors after treatment with IL13Rα2 CAR T cells or IL13Rα2-IFNγ CAR T cells. 28C. Bar graph showed tumor progression in mice bearing medium-IL13Rα2 antigen expressing tumors after treatment with IL13Rα2 CAR T cells or IL13Rα2-IFNγ CAR T cells.

FIGS. 29A-29D: IL13Rα2-IFNγ CAR T cells and IL13Rα2-IFNγ^{low} CAR variants expressed different levels of IFNγ. 29A Schematics depicting the construct designs with IFNγ under the control of different strength promoters. 29B. Schematic depicting the experimental design showing transduction and collection of supernatant. 29C. Bar graph showed IFNγ levels during ex vivo expansion. 29D. Bar graph showed viable tumor counts after co-culture of CAR T cells with tumor (1:50 effector:target ratio) for 5 days.

FIGS. 30A-30C: IL13Rα2-CAR T and IL13Rα2-CAR-NFAT/IFN [symbol] T cells show comparable killing capacity. 30A. Schema of construct design demonstrates IFN [symbol] under the control of the NFAT promoter. 30B. Schematic of the experimental design demonstrated coculture of different CARs in the presence of antigen positive tumors. 30C. Graph demonstrates cytotoxic function of IL13Rα2-CAR T compared to IL13Rα2-CAR-NFAT/IFN [symbol] T cells.

FIGS. 31A-31B: IL13Rα2-IFNγ CAR T cells synergizes with myeloid cells for an enhanced antitumor function. 32A. Schema depicting the experimental design; briefly, T cells transduced with IL13Rα2-IFNγ CAR T cells or IL13Rα2 CAR T cells were co-cultured with macrophages and antigen positive tumor cells. 32B. Bar graph showed tumor count in the 3-way co-culture with IL13Rα2-IFNγ CAR T cells or IL13Rα2 CAR T cells with or without macrophages or tumor cells.

### DETAILED DESCRIPTION

### Examples

The invention is further described in the following examples, which do not limit the scope of the invention described in the claims.

### Materials and Methods

### Mice and Cell Lines

C57BL/6/J, CD45.1 (B6.SJL-Ptprc^{a}Pepc^{b}/BoyJ), Thy1.1 (B6.PL-Thy1a/CyJ), IFNγR−/− (B6.129S7-Ifngr1tm1Agt/J), and IFNγ−/− (B6.129S7-Ifngtm1Ts/J) mice were purchased from The Jackson Laboratory. NOD/Scid IL2RγCnull (NSG) mice were bred at City of Hope. All mouse experiments were approved by the City of Hope Institutional Animal Care and Use Committee (IACUC).

The luciferase-expressing murine GL261 (GL261-Luc) and KR158B (K-Luc) glioma cells were transduced with lentivirus to produce murine IL13Rα2 (mIL13Rα2) expressing sublines (GL261-Luc-mIL13Rα2 and K-Luc-mIL13Rα2). These tumor lines were maintained in DMEM (Gibco) supplemented with 10% fetal bovine serum (Hyclone Laboratories), 25 mM HEPES (Irvine Scientific, Santa Ana, CA) and 2 mM L-glutamine (Lonza). Cell surface expression of mIL13Rα2 was authenticated by flow cytometry and immunofluorescence imaging.

Patient-derived glioma cells (PBT030-2-ffLuc) were isolated from GBM patient resections under protocols approved by the COH IRB and maintained as described previously. All tumor lines were authenticated for the desired antigen/marker expression by flow cytometry and cells were tested for mycoplasma and maintained in culture for less than 1-3 months.

### CAR T Cell Production

Human CAR T cells: Naive and memory T cells were isolated from healthy donors at City of Hope under protocols approved by the COH IRB (12, 32). The construct of IL13Rα2-targeted CAR and CAR transduction was described in previous studies (12, 33, 34). In brief, primary T cells were stimulated with Dynabeads Human T expander CD3/CD28 (Invitrogen) (T cells: beads=1:2) for 24 hours and transduced with CAR lentivirus (multiplicity of infection [MOI]=0.5). Seven days after CAR transduction, CD3/CD28 beads were removed and cells were resuspended and expanded in X-VIVO 15 media (Lonza) containing 10% FCS, 50 U/ml recombinant human IL-2, and 0.5 ng/ml recombinant human IL-15 for additional 10-15 days before proceeding to ex vivo expansion.

### Murine CAR T Cells

The murine IL13BBζ chimeric antigen receptor was constructed in a MSCV retroviral backbone (Addgene), containing the extracellular murine IL13 and murine CD8 hinge, murine CD4 transmembrane domain, and intracellular murine 4-1BB costimulatory and murine CD3ζ signals. Following a T2A ribosomal skip, a truncated murine CD19 was inserted as a transduction marker. The resulting plasmid was transfected into PlatE cells (a gift from Dr. Zuoming Sun lab) using Fugene (Promega). After 48 hours, the supernatant was collected and filtered using an 0.2 μm filter. The retroviral supernatant was aliquoted and frozen until the time of transduction.

Murine T cells were isolated from spleens of naïve C57BL6J mice or appropriate strain (CD45.1, Thy1.1, or IFNγ−/−) with EasySep Mouse T cell Isolation Kit (STEMCELL Technologies) and stimulated with Dynabead Mouse T-Activator CD3/CD28 beads (Gibco) at a 1:1 ratio. T cells were transduced on RetroNectin-coated plates (Takara Bio USA) using retrovirus-containing supernatants (described above). Cells were then expanded for 4 days in RPMI-1640 (Lonza) supplemented with 10%/6 FBS (Hyclone Laboratories), 55 mM 2-mercaptoethanol (Gibco), 50 U/mL recombinant human-IL-2 (Novartis), and 10 ng/mL recombinant murine IL-7 (Peprotech). Before in vitro and in vivo experiments, T cells were debeaded and CAR expression was determined by flow cytometry.

qRT-PCR Analysis

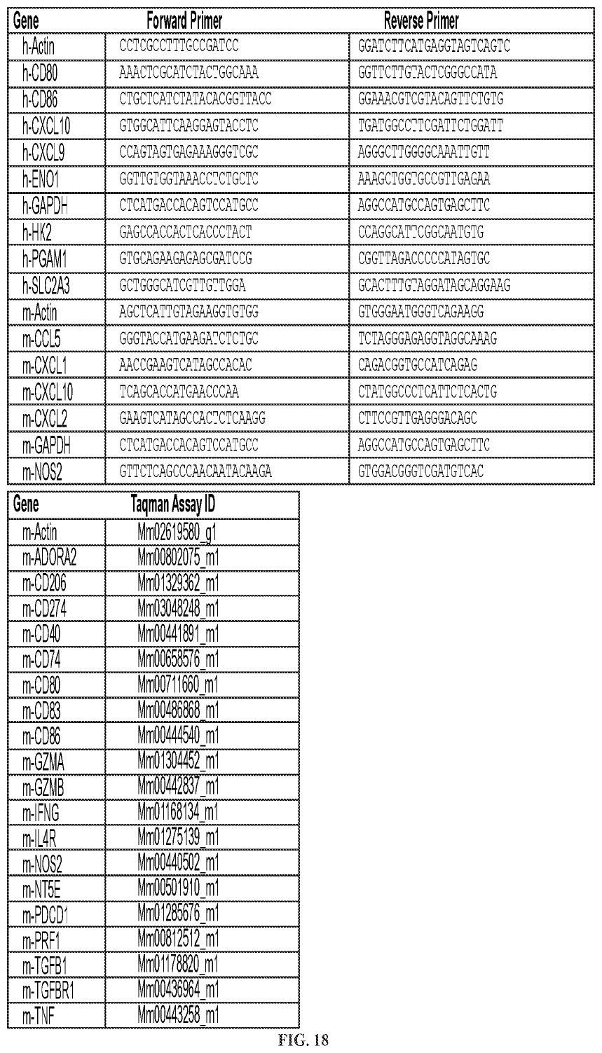

RNA was isolated from myelin-removed brain tissue (either bulk tissue or flow sorted cells) using the RNeasy Mini Kit (Qiagen). cDNA was reverse transcribed using the SuperScript VILO Mastermix (Life Technologies) according to the manufacturer's instructions. qPCR reactions were performed as previously described (35). Primers are used are listed in FIG. 18.

### In Vivo Studies

All mouse experiments were performed using protocols approved by the City of Hope IACUC. Orthotopic GBM models were generated as previously described (36). Orthotopic tumor model was established by stereotactically implanting 1-105 tumor cells intracranially (i.e.) into the right forebrain of 8-10 week-old C57BL/6J, IFNγR−/−, or NSG mice. Engraftment was verified by bioluminescent imaging one day prior to CAR T cell injection, Mice were randomized into groups based on bioluminescent signal. Four or seven days after tumor injection, mice were treated intracranially with 1×10⁶ mIL13BBζ-CAR T cells. Tumor burden was monitored with SPECTRAL LagoX (Spectral Instruments Imaging) and analyzed using Aura software (v2.3.1, Spectral Instruments Imaging). Survival curves were generated by GraphPad Prism Software (v8).

For rechallenge experiments, clearance of tumor was verified by bioluminescent imaging prior to tumor rechallenge, where mice were injected with 10⁴ K-Luc or 5×10⁴ GL261-Luc cells. For subcutaneous studies, 1×10⁶ K-Luc-mIL13Rα2 in PBS was injected into the right and left flanks of 8-10 week-old C57BL/6J donor mice. Tumors were allowed to establish for 8 days, then 1×10⁶ CAR T cells were injected directly into the tumor. Three days later, the tumor mass were harvested, manually dissociated and sorted by flow cytometry into CD3+CD19-(endogenous T cells) or CD3+CD19+(CAR T cells) using the BD AriaSORP (BD Biosciences). The purified T cell populations were either used as effector cells in in vitro coculture 10:1 (effector:target) ratio as described below or reinjected back into 8 day old subcutaneous K-Luc-mIL13Rα2 tumors, which tumor volume was measured over time using calipers.

Mice were also monitored by the Center for Comparative Medicine at City of Hope for survival and any symptoms related to tumor progression, with euthanasia applied according to the American Veterinary Medical Association Guidelines.

### In Vitro Cytotoxicity

For assessment of CAR T cell proliferation and cytotoxic activity, K-Luc-mIL13Rα2 or GL261-Luc-mIL13Rα2 tumor cells were co-cultured with murine CAR T cells at 1:3 CAR+ tumor ratio for 48 hours. For co-culture using effector T cells primed in vivo, T cells were plated at a 10:1 effector: tumor ratio for 72 hours. Cells were stained with anti-CD3, CD8, and CD19. Absolute number of viable tumor and CAR T cells was assessed by flow cytometry.

For the degranulation assay, CAR T cells and tumor cells were co-cultured at 1:1 effector: tumor ratio for 5 hours in the presence of GolgiStop Protein Transport Inhibitor (BD Biosciences). The cell mixture was stained with anti-CD3, CD8, and CD19 followed by intracellular staining with anti-IFNγ (BD Biosciences), GZMB and TNFα (eBiosciences) antibodies and analyzed by flow cytometry.

All samples were acquired on MACSQuant Analyzer (Miltenyi Biotec) and analyzed with FlowJo software (v10.7) and GraphPad Prism (v8).

### Patient Sample Analysis

Conditioned media was generated by seeding patient-derived glioma cells, human CAR T cells, or the combination at a 1:1 ratio for 24 hours. The supernatant was collected and centrifuged to remove any cell debris. Peripheral blood from GBM patients (obtained from scheduled blood draws under clinical protocols approved by the City of Hope) was lysed with PharmLyse buffer (BD Biosciences). CD3 and CD14 cells were isolated using selection kits (STEMCELL Technologies). CD14 and CD3 positive cells were incubated with conditioned media, in the presence or absence of IFNγR neutralizing antibody (R&D Systems). For macrophage differentiation, CD14 cells were incubated in the presence of M-CSF (BioLegend) for 7 days and then exposed to conditioned media, in the presence or absence of IFNγR neutralizing antibody (R&D Systems). After 48 hours, cells were visualized using Keyence microscope and phenotyped by flow cytometry.

Assessment of endogenous response in the unique responder to CAR T therapy (ref) was conducted as previously reported (37). Briefly, T cells were isolated from total blood before and during therapy. Every two days, T cells were incubated with irradiate (40 Gy) autologous tumor cells in the presence of IL2 (50U/ml). After 14 days, T cells were purified and counted. T cells were cultured with fresh autologous tumor or irrelevant tumor line at a 10:1 (effector:target) ratio after 3 days, tumor counts were measured. IFNγ production was measure by stimulating the T cells with cell stimulation cocktail for additional 4 hours followed by flow cytometry for intracellular IFNγ.

### Flow Cytometry Assays

Live tumor cells expanded in vitro were stained with an unconjugated goat anti-mouse IL13Rα2 (R&D Systems) followed by secondary donkey anti-goat NL637 (R&D Systems). Live murine CAR T cells were stained with CD8 (BioLegend) CD3, CD4, CD62L (eBiosciences) or CD45RA (BD Biosciences). CD19 (BD Biosciences) was used as a surrogate to detect the CAR.

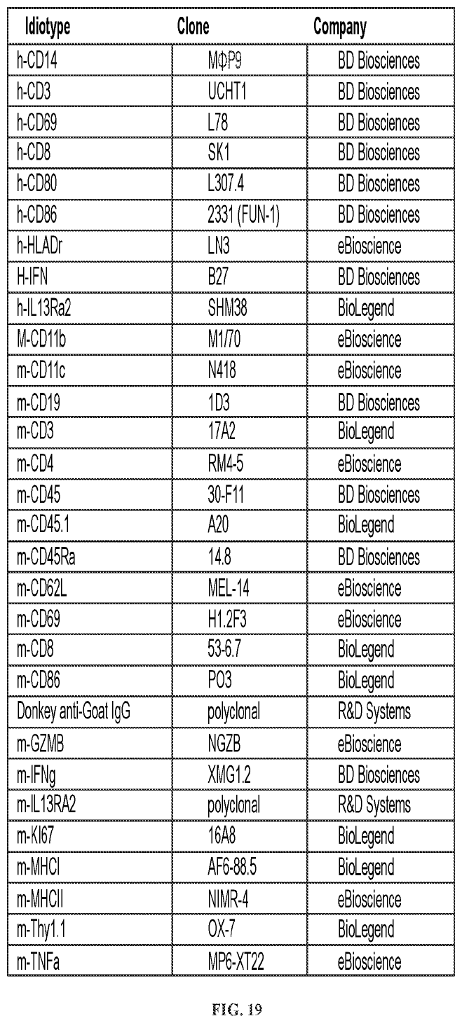

Brains from euthanized mice were removed at the indicated time-points, and a rodent brain matrix was used to cut along the coronal and saggital planes to obtain a 4×4 mm section, centered around the injection site. These sections were minced manually, then passed through a 40 μm filter. Myelin was removed using Myelin Removal Beads II and LS magnetic columns (Miltenyi Biotec) according to the manufacturer's instructions, then cells were counted. Cell were stained and analyzed using flow cytometry. For flow sorting, cells were stained with indicated antibodies (FIG. 19) and sorted using BD AriaSORP (BD Biosciences). For gene expression analysis of TME, the remaining cells were lysed for RNA.

### Immunofluorescence and Immunohistochemistry

For immunofluorescence, K-Luc and GL261-Luc parental or mIL13Rα2-transduced cells were cultured on coverslip, stained with unconjugated goat anti-mouse IL13Rα2 (R&D Systems) followed by secondary donkey anti-goat NL637 (R&D Systems), and actin. Slides were imaged using confocal microscopy (Zeiss confocal microscopy) as previously described (38).

For immunohistochemistry, mice were euthanized 3 days after CAR T injection and were perfused with PBS followed by 4% PFA. Whole brains were dissected, and incubated in 4% PFA for 3 days, followed by 70% ethanol for 3 days before being embedded in paraffin. 10 μM transverse sections were cut and stained with H&E, CD3 (ab16669, Abcam) or F4/80 (ab6640, Abcam). Slides were digitized at 40× magnification using a NanoZoomer 2.0-HT digital slide scanner (Hamamatsu).

### Bioplex Cytokine Analysis

To assess CAR T cell cytokine profile, mIL13BBζ CAR+ T cells and tumor cells (GL261-Luc-mIL13Rα2 or K-Luc-mIL13Rα2) were incubated at 1:1 ratio for 1 day without exogenous cytokines. The cell-free supernatant was collected and assayed using the ProcartaPlex Mouse Th1/Th2 Cytokine Panel 11plex (ThermoFisher Scientific) according to the manufacturer's instructions and acquired on the Bio-Plex 3D Suspension Array System (Bio-Rad Laboratories).

### Nanostring Gene Expression Analysis

RNA was purified from flow-sorted CD3+ or CD11b+ sorted cells using the RNEasyPlus micro kit, following the manufacturer's instructions (Qiagen, Germantown, MD, USA). RNA samples were subsequently quantified and qualified using Nanodrop 1000 spectrophotometer (ThermoFisher, Waltham, MA, USA) and Bioanalyser Tape station (Agilent, Santa Clara, CA, USA) assays. The subsequent Nanostring analysis was performed at concentrations of 35ng/well and 25ng/well respectively for CD3+ cells and CD11b+ cells.

Samples were analyzed based on the nCounter® mouse PanCancer Immune profiling gene expression panel (NanoString Technologies, Seattle, WA, USA): Hybridation reaction was performed for 18h at 65° C. Fully automated nCounter FLEX analysis system; composed of an automated nCounter® Prep station and the nCounter® Digital Analyzer optical scanner (NanoString Technologies, Seattle, WA, USA) was used. Normalization was performed by using the geometric mean of the positive control counts as well as normalization genes present in the CodeSet Content: samples with normalization factors outside of the 0.3-3.0 range were excluded, samples with reference factors outside the 0.10-10.0 range were excluded as well. Gene expression analysis was performed using the nSolver v3.0 and Advanced analysis module softwares. (NanoString Technologies, Seattle, WA, USA).

### Single Cell RNA-Sequencing

Seven days after K-Luc-mIL13Rα2 engraftment, CAR T cells were injected or not into the tumor as described above. Brains from CAR T treated or untreated mice (n=3 per group) were harvested and pooled three days after CAR T cell injection, manually minced, and myelin removed before flow sorting on the BD AriaSORP (BD Biosciences) for live (DAPI−) CD45-PE+ (BD Biosciences) cells. Single cell suspensions were processed using the Chromium Single Cell 3′ v3 Reagent Kit (10× Genomics) and loaded onto a Chromium Single Cell Chip (10× Genomics) according to the manufacturer's instructions. Raw sequencing data from each of two experiments were aligned back to mouse genome (mm10), respectively, using cellranger count command to produce expression data at a single-cell resolution according to 10× Genomics (https://support.10xgenomics.com/single-cell-gene expression/software/pipelines/latest/using/count).R package Seurat 39 was used for gene and cell filtration, normalization, principle component analysis, variable gene finding, clustering analysis, and Uniform Manifold Approximation and Projection (UMAP) dimension reduction. Briefly, matrix containing gene-by-cell expression data was imported to create a Seurat object individually for CAR T untreated and CAR T treated samples. Cells with <200 detectable genes and a percentage of mitochondrial genes >10% were further removed. Data were then merged and log-normalized for subsequent analysis. Principle component analysis (PCA) was performed for dimension reduction, and the first 20 principle components were used for clustering analysis with a resolution of 0.6. Clusters were visualized with UMAP embedding. In additional to the use of Immunologic Genome Project (ImmGen)40, 41, to facilitate cell type identification, the expression level of the following markers were plotted using VInPlot. They were ltagm, Cd3e, Cd19, Cd79a, Nkh7, Cd68 and Cd8a. Upon the identification of lymphoid and myeloid parental clusters, on each of them, we followed the above-mentioned strategy for subclustering to produce daughter clusters. In concert with ImmGen, key markers for distinguishing myeloid daughter clusters were Itgam, Cd68, S100a9, Itgax, Tmem119, and P2ry12, while for lymphoid Cd3e, Cd4, Cd8a, Cd79a, and Ncr1. To further visualize the average expression of a module of genes, CD74, H2-Aa, H2-Ab1, H2-Eb1, and MARCKS, across population in myeloid daughter clusters, AddModuleScore function was employed to generate a feature that could be rendered using FeaturePlot.

### Gene Set Enrichment Analysis

Differentially expressed (DE) genes between untreated and CAR T treated in each myeloid and lymphoid parental and daughter cluster were detected with function FindAllMarkers. The analysis on Gene ontology (GO), Kyoto encyclopedia of genes and genomes pathway, and Immunologic signatures collection (ImmuneSigDB) (42) was performed with the full list of DE genes of each cluster using GSEA function implemented in clusterProfiler package (43), then being plotted with ggplot2 (H. Wickham. ggplot2: Elegant Graphics for Data Analysis. Springer-Verlag New York, 2016).

### Statistical Analysis

Statistical significance was determined using Student t-test (two groups) or one-way ANOVA analysis with a Bonferroni (three or more groups). Survival was plotted using a Kaplan-Meier survival curve and statistical significance was determined by the Log-rank (Mantel-Cox) test. All analyses were carried out using GraphPad Prism software (v5). *, P<0.05; **, P<0.01; ***, P<0.001.

### Example 1: Murine IL13Rα2-CAR T Cells Mediate Potent Antitumor Activity in Immune Competent Models of GBM

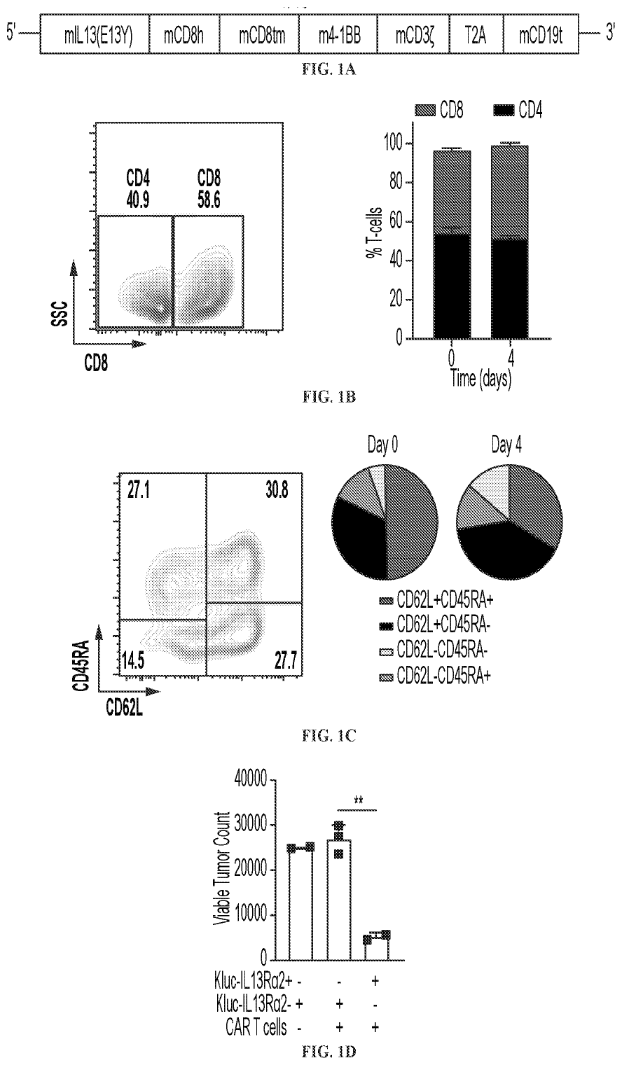

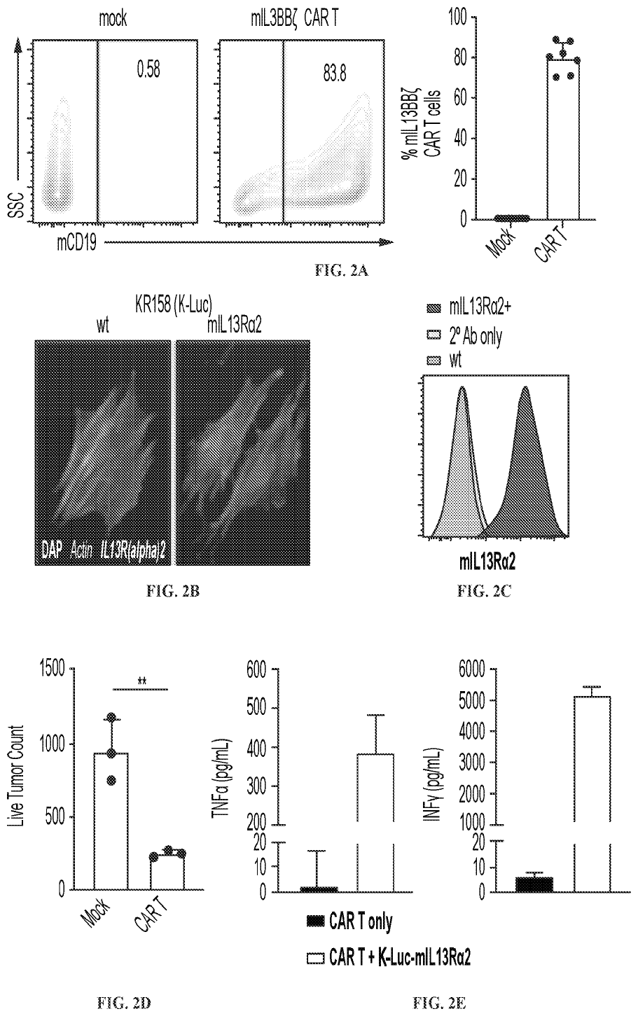

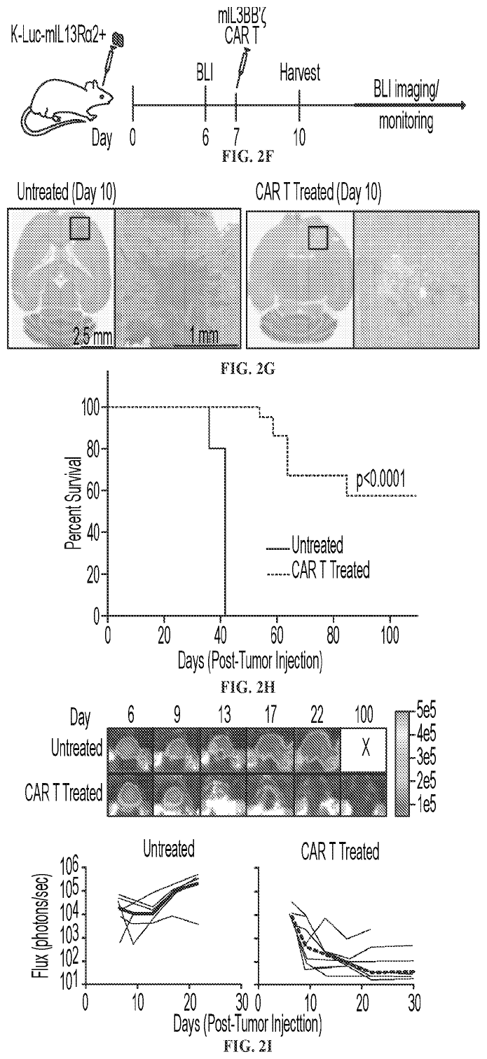

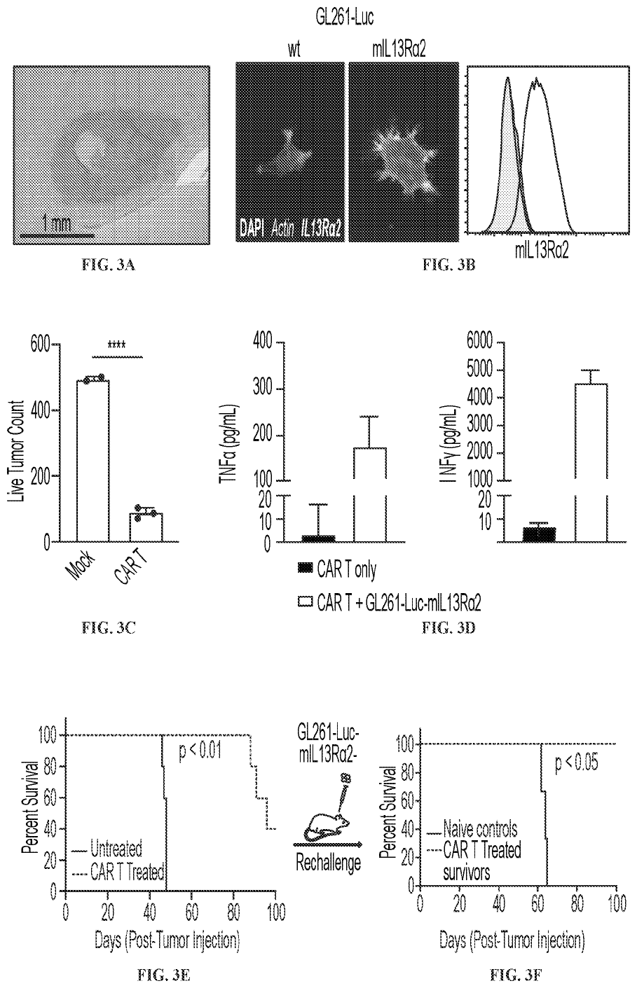

We established immunocompetent mouse models of an earlier described clinical IL13Rα2-CAR T cell platform. A mouse counterpart to a human IL13Rα2-targeted CAR was constructed (12), composed of the IL-13(E12Y) tumor-targeting domain, murine CD8 hinge (mCD8h), murine CD8 transmembrane domain (mCD8tm), murine 4-1BB costimulatory domain (m4-1BB) and murine CD3 zeta (mCD3ζ). A T2A skip sequence separates the CAR from a truncated murine CD19 (mCD19t) used for cell tracking (FIG. 1A). The engineering process resulted in a 70-85% transduction efficiency as assessed by the frequency of CD19t+ cells (FIG. 2A). Phenotypic analyses indicated that, similar to human CAR T cells (12), the murine IL13Rα2-targeted CAR T cells (mIL13BBζ CAR T cells) contained comparable numbers of CD4+ and CD8+ T cell subsets with mixed early memory (CD62L+) and effector (CD62L−) T cell populations on day 4 of ex vivo expansion (FIG. 1B and FIG. 1C). We designed a therapeutic setting based on K-Luc and GL261-Luc, two syngeneic, immunocompetent murine glioma models. K-Luc, a firefly luciferase engineered subline of KR15813, was used as it recapitulates the highly invasive features of GBM (FIG. 2B). This tumor line is derived from a spontaneous glioma arising from Nf1, Trp53 mutant mice, and is poorly immunogenic as indicated by its unresponsiveness to anti-PD-1 checkpoint therapy (14). As a second model, we also used GL261 engineered to express ffLuc (GL261-luc), a non-invasive “bulky” glioma (FIG. 3A), which was generated by chemical induction, contains high numbers of mutations, and in contrast to K-Luc, is responsive to anti-PD-1 immunotherapy (15, 16). Both tumor lines were engineered to express murine IL13Rα2 (mIL13Rα2) (FIG. 2C and FIG. 3B). mIL13BBζ CAR T cells specifically killed mIL13Rα2-engineered K-Luc and GL261-Luc cells (FIG. 2D and FIG. 3C), which was associated with production of inflammatory cytokines IFNγ and TNFα (FIG. 2E and FIG. 3D), and were not responsive to IL13Rα2-negative parental tumor lines in vitro (FIG. 1D).

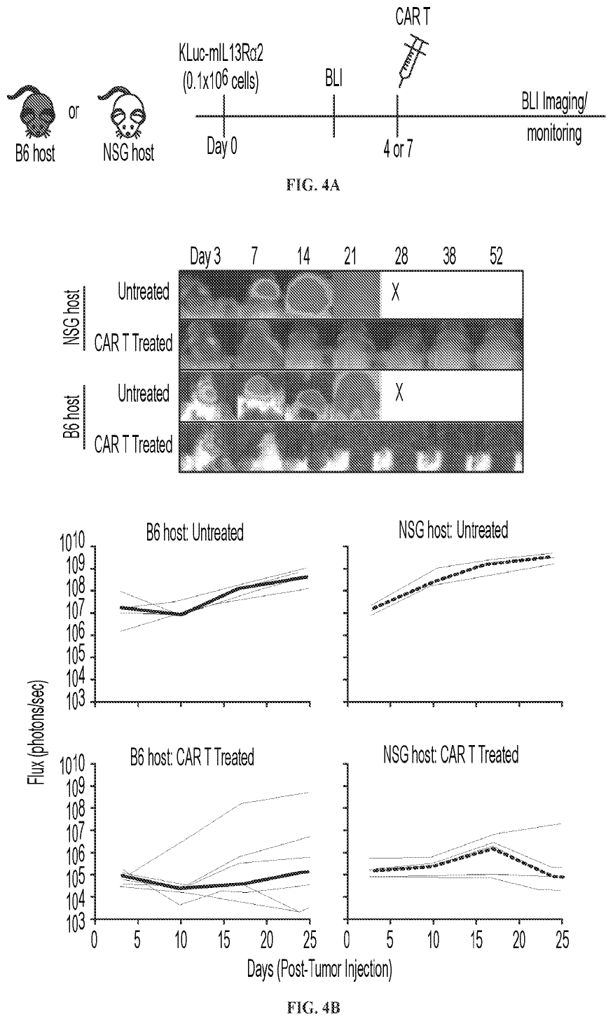

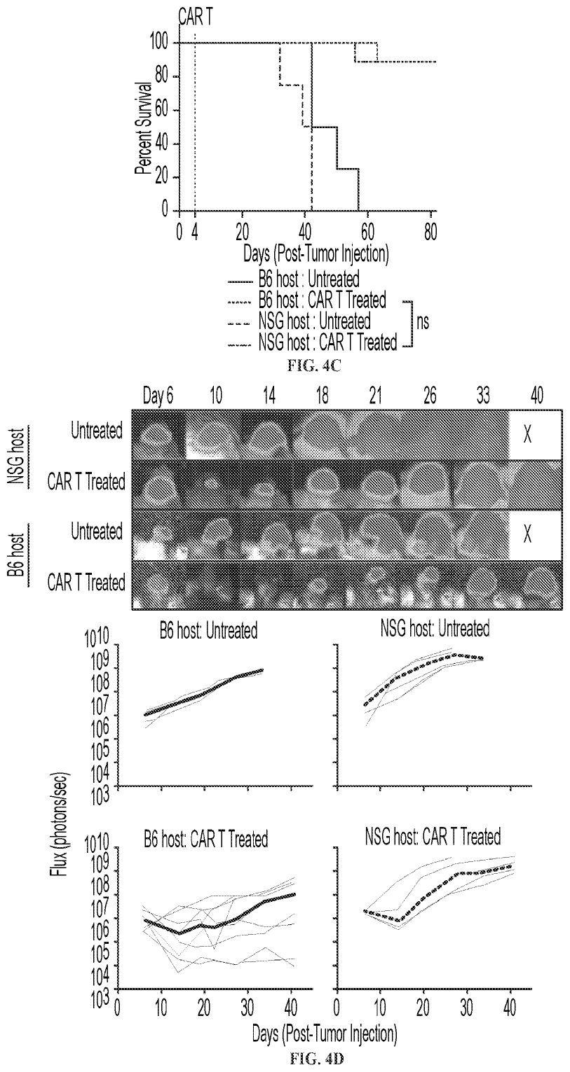

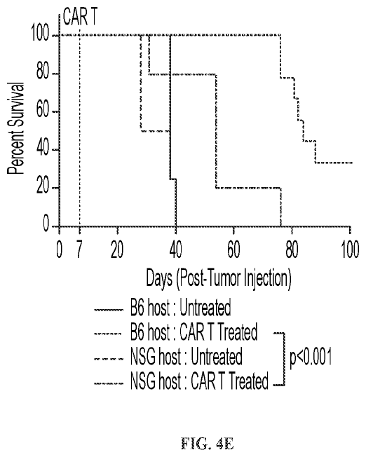

We next evaluated CAR T cell antitumor activity against orthotopically engrafted glioma tumors in C57BU/6 immunocompetent mice. In IL13Rα2+K-Luc and GL261-Luc tumor models, single intratumoral administration of mIL13BBζ CAR T cells seven days after tumor injection mediated potent in vivo antitumor activity and conferred a significant survival benefit (FIGS. 2F-2I and FIG. 3E). We compared anti-K-Luc activity in C57BL/6 mice to tumors engrafted in immunocompromised NOD-scid IL2Rgnull (NSG) models, which lack adaptive immune subsets. In a smaller four-day old tumor model, antitumor activity in C57BL/6 and NSG was equivalent, indicating CAR T cell functionality is comparable in both mouse strains (FIGS. 4A-4C). However, surprisingly, in the larger tumor model (day 7), with a more established immune microenvironment, the in vivo response to T cell therapy was superior in immunocompetent C57BL′6 as compared to immunodeficient NSG mice (p<0.001) (FIGS. 4D-4E). This observation raised the possibility that tumor infiltrating immune cells may enhance the antitumor effects of CAR T cell therapy in GBM.

### Example 2: CAR T Therapy can Promote Immunological Memory and Generation of Tumor-Specific T Cells

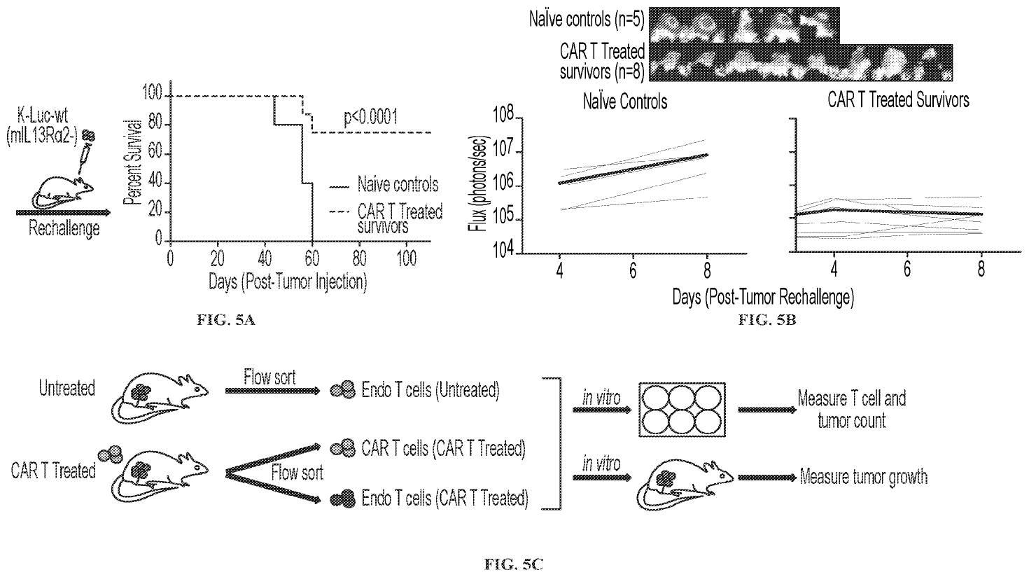

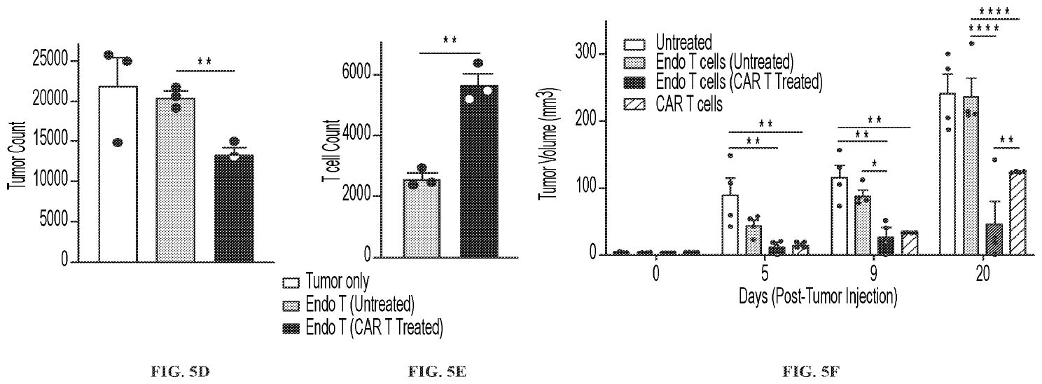

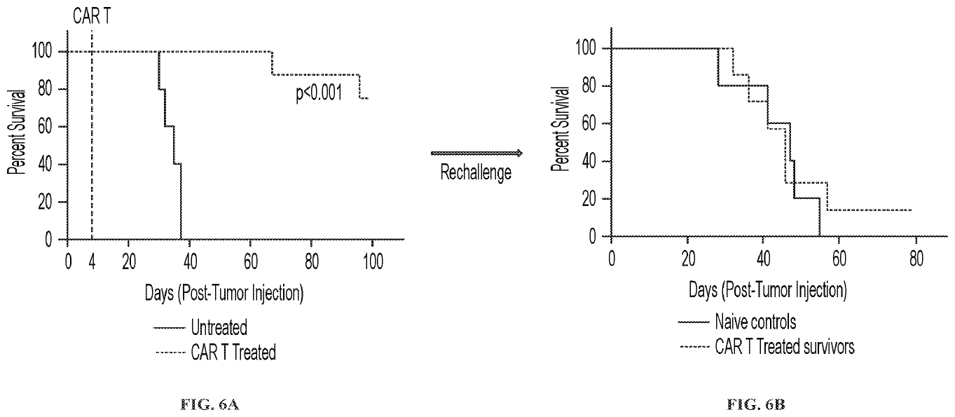

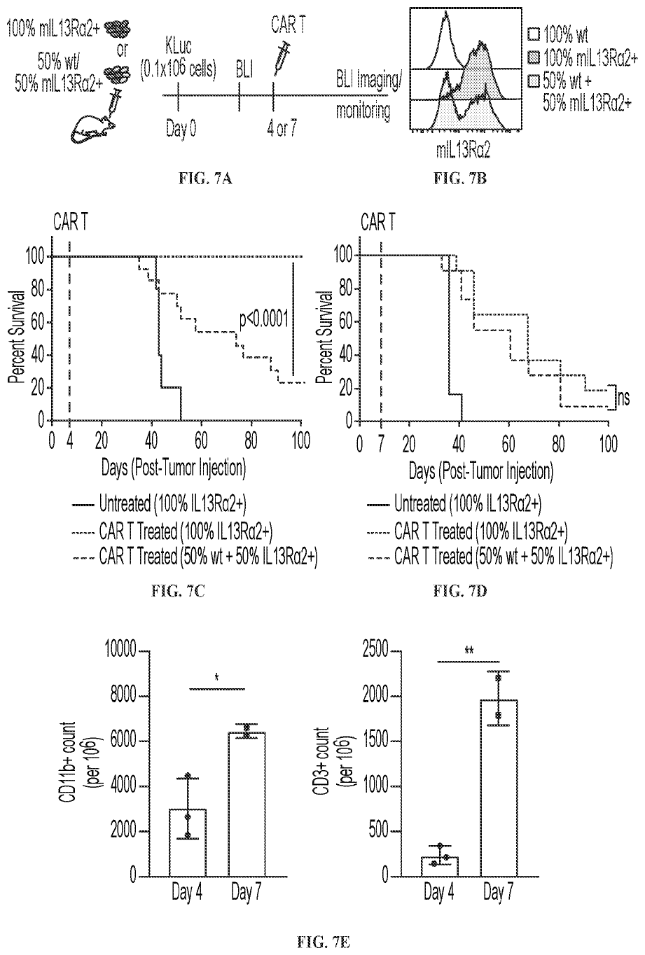

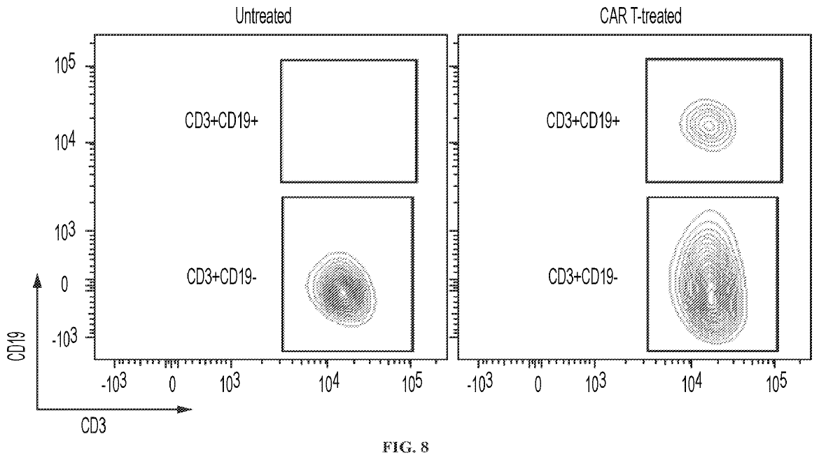

To evaluate whether CAR T cells have the potential to induce endogenous antitumor immunity, cured mice following CAR T cell treatment were challenged with IL13Rα2-negative parental tumors. Indeed, in the larger engrafted tumors (7 day engraftment before CAR T therapy), cured mice in the immunocompetent C57BL16 model successfully rejected tumor rechallenge with IL13Rα2-negative K-Luc (FIG. 5A and FIG. 5B) and GL261-Luc (FIG. 3F) parental tumor cells, demonstrating that CAR T cells can promote immunological memory in two independent tumor models with differential responsiveness to anti-PD1 immunotherapy (14, 16). The capacity of CAR T cells to induce endogenous immunity against IL13Rα2-negative tumor cells again required a more established TME, as mice cured in the small tumor model (day 4) were not capable of mounting antitumor responses following rechallenge with parental tumors (FIG. 6). This observation also suggests that tumor exposure is not sufficient to induce endogenous antitumor immunity, and instead the establishment of immunological memory requires both CAR T cell therapy and the host immune infiltrates. In a similar set of experiments, we compared the antitumor activity of mIL13BBζ CAR T cells against IL13Rα2+K-Luc (100% IL13Rα2+ cells) versus a mixture of IL13Rα2+K-Luc and IL13Rα2− parental K-Luc tumor cells (1:1 ratio). Supporting the notion that CAR T cells can promote endogenous immune responses against antigen-negative tumor cells, mIL13BBζ CAR T cells mediated comparable survival benefit against tumors with homogenous and heterogeneous IL13Rα2-antigen expression (p<0.001; FIG. 7A and FIG. 7B). Again, the response to IL13Rα2-negative tumors required a more established tumor microenvironment, as CAR T cell therapy was less effective against mixed antigen tumors (1:1 ratio) in the small tumor model (day 4) (p<0.001; FIG. 7C and FIG. 7D). Together these observations suggest that the TME of GBM can be altered by CAR T cell therapy to augment antitumor responses and promote endogenous antitumor memory responses. To more directly evaluate whether CAR T therapy can potentiate the generation of tumor-specific T cells, we isolated intratumoral endogenous (CD3+CD19−) and CAR T cells (CD3+CD19+) from untreated and CAR T cell treated mice in the IL13Rα2+K-Luc glioma model (3 days post treatment) (FIG. 8), and either ex vivo co-cultured with tumor cells or adoptively transferred into tumor bearing mice (FIG. 5C). In vitro, endogenous T cells isolated from CAR T cell treated tumors, but not control untreated groups, exhibited enhanced killing of IL13Rα2+K-Luc cells and T cell proliferation in co-culture assays (10:1, effector:target ratio; 72 hours) (FIG. 5D and FIG. 5E). To assess in vivo function, endogenous T cells from untreated and CAR T cell treated mice were isolated and adoptively transferred into IL13Rα2+K-Luc tumor bearing mice. Measurement of tumor progression demonstrated that mice injected with endogenous T cells isolated from CAR T treated mice showed a significant reduction in tumor growth compared to the control group (FIG. 5F). Collectively, these results establish that CAR T cells have the potential to promote antigen spread and the generation of tumor-specific T cell responses.

### Example 3: CAR T Cells Activate Innate and Adaptive Immune Subsets in Tumor Microenvironment

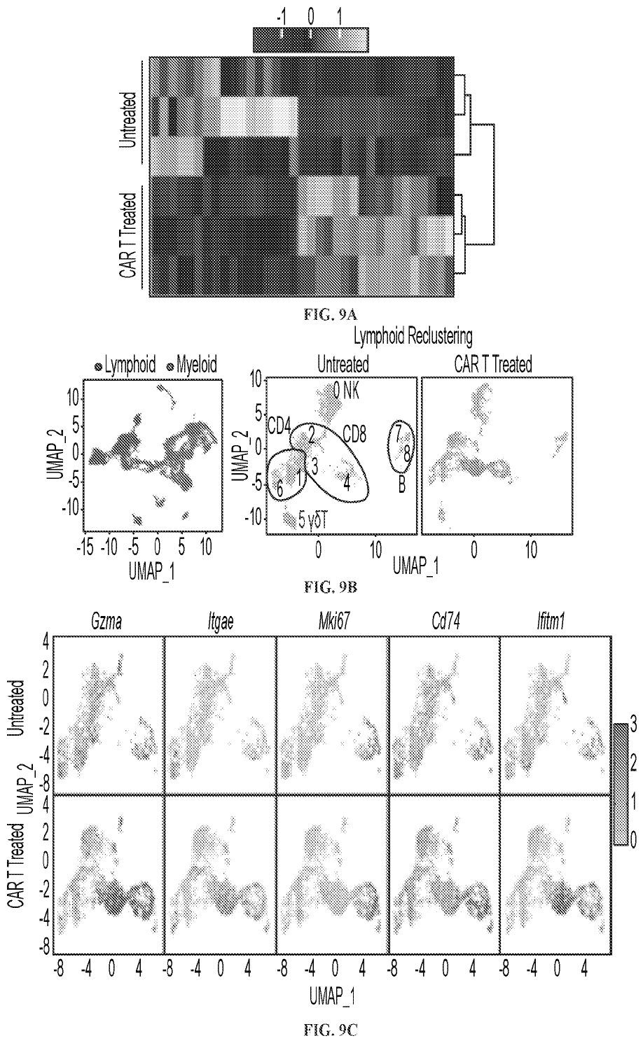

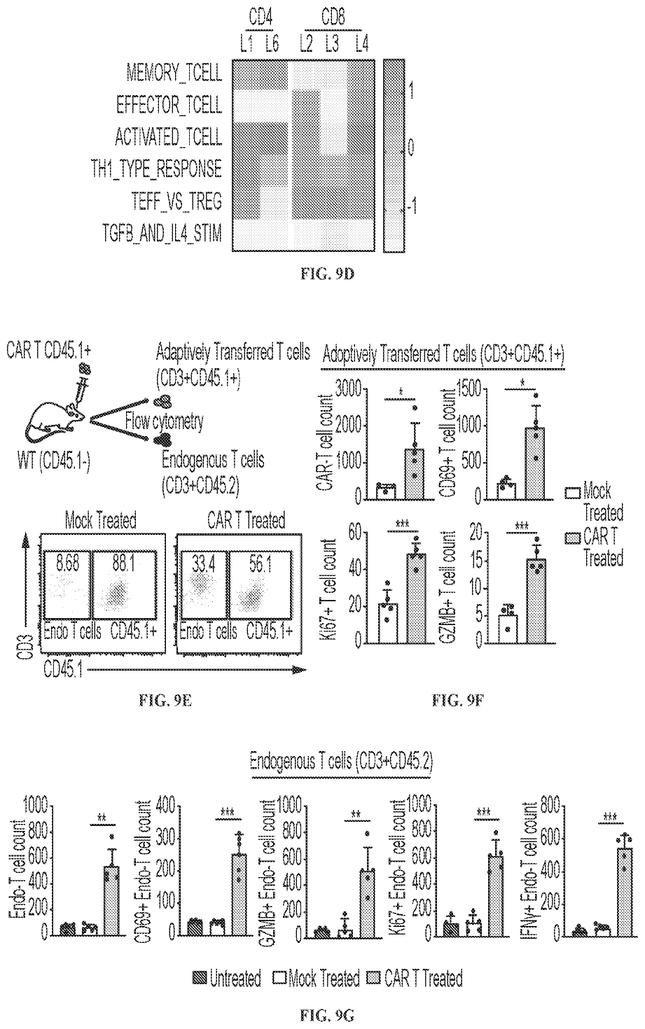

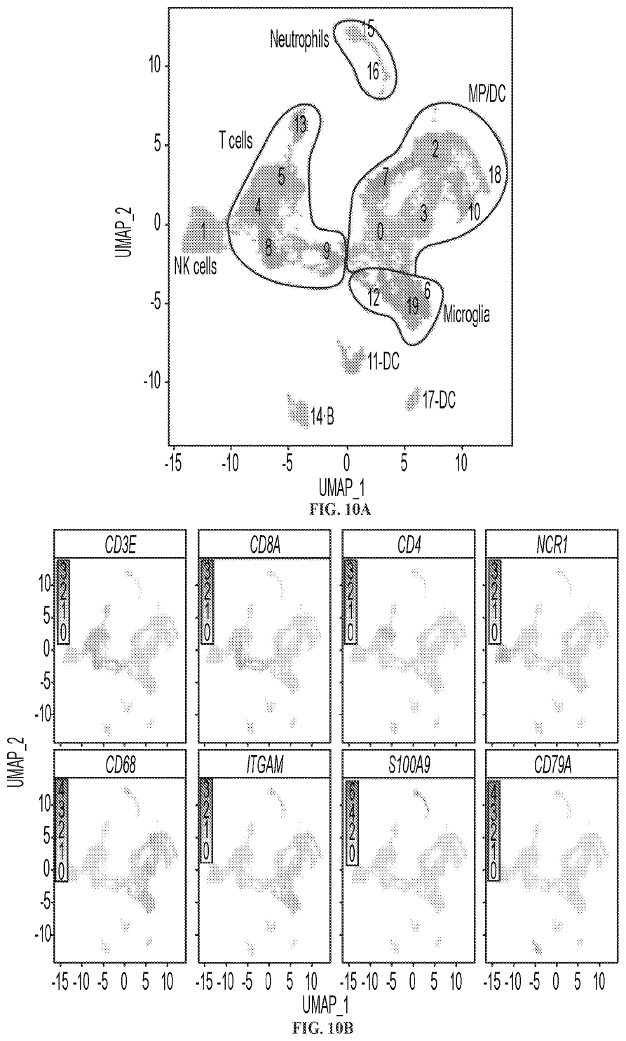

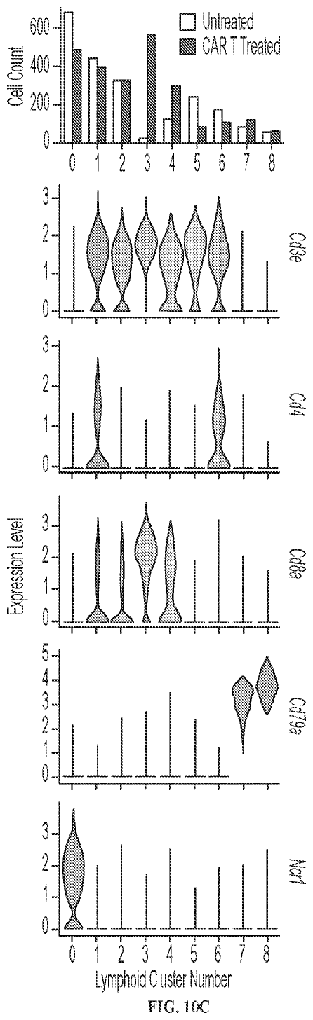

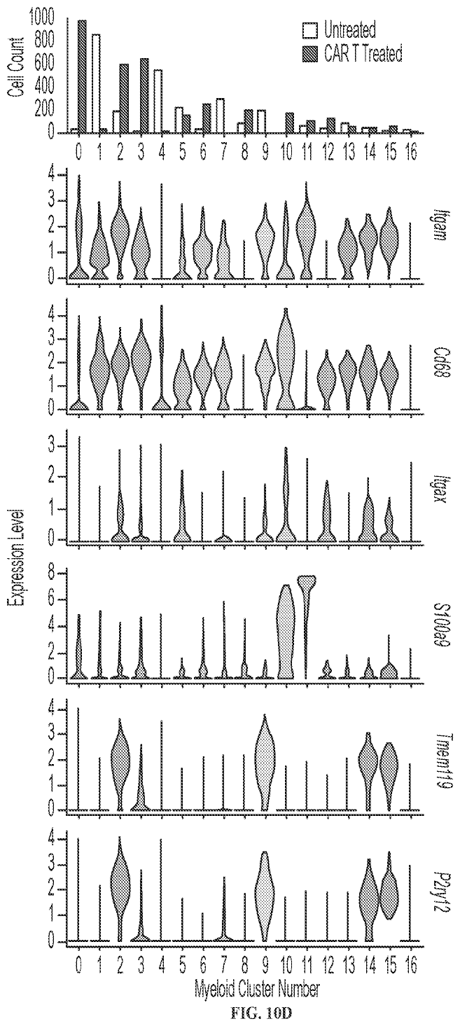

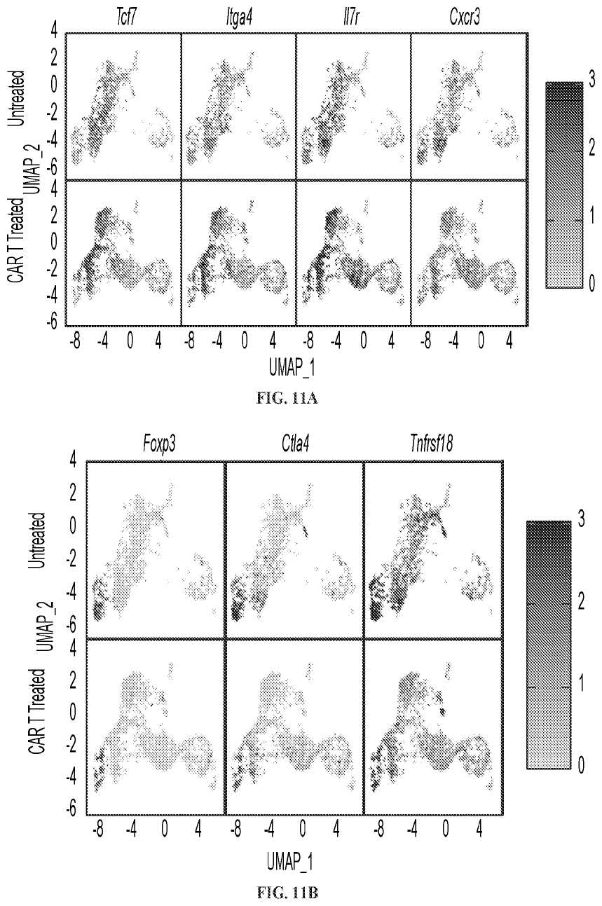

To elucidate immune-related changes in the TME that coincide with the establishment of endogenous antitumor immunity following CAR T cell therapy, we interrogated both the lymphoid and myeloid compartments by gene and protein expression profiling. Focusing first on the lymphoid compartment, we performed nanostring analysis of purified intratumoral CD3+ cells and demonstrated global changes at transcriptome level in CAR T treated mice compared to untreated (FIG. 9A). To increase resolution and more accurately define subpopulations, we performed scRNAseq on isolated CD45 cells from untreated or CAR T treated mice (FIG. 10A and FIG. 10B). We then computationally separated lymphoid and myeloid populations and reanalyzed the scRNAseq data at higher granularity. This approach yielded nine distinct lymphoid subpopulations broadly defined by the distribution of classical marker genes (FIG. 10C), including three distinct subsets of CD8+ T cells (CD8_L2, CD8_L3, and CD8_L4), two subsets of CD4+ T cells (CD4_L1, CD4_L6), one subset of NK cells, two subsets of B cells and one subset resembling γδT cells (FIG. 9B and FIG. 10C). The frequency of CD8_L2 remained unchanged, but interestingly, post CAR T therapy, increased frequency of CD8_L3 and CD8_L4 subclusters were detected FIG. 10C). Focusing on T cell subclusters, CD8_L3 is observed mainly post therapy and is characterized by expression of Cxcr3 (FIG. 11a) which is associated with T cell trafficking and expression of Itgae (CD103), Cd74 (Hladr) and Iftm1 (IFN-induced transmembrane protein 1) that correspond to activated resident memory CD8 T cell phenotype (FIG. 9C). CD8 L4 expanded post-therapy and was identified as highly activated, effector T cells based on higher expression of Ki67, Cd74, and Gzma genes (FIG. 9C). Within the CD4 subsets, the frequency of CD4_L1 cluster remained unchanged after therapy, it displayed a modest increase in expression of II7r, Tcf7, and ltga4 genes which is associated with effector memory CD4 T cells in CAR T treated group (FIGS. 10C and 11A). Intratumoral regulatory T cells (Treg), defined by subcluster CD4_L6 based on the expression of CD4, Foxp3, GITR (Tnfrsf18) and Ctla4, decreased after CAR T therapy (FIG. 11B). Overall, gene set enrichment analysis (GSEA), revealed enrichment of gene signatures associated with activated and Th1 responses in most T cells subclusters (FIG. 9D). Taken together, these studies establish that CAR T cells can dramatically alter the lymphoid compartment within tumors and result in clusters of activated, memory or effector T cell populations.

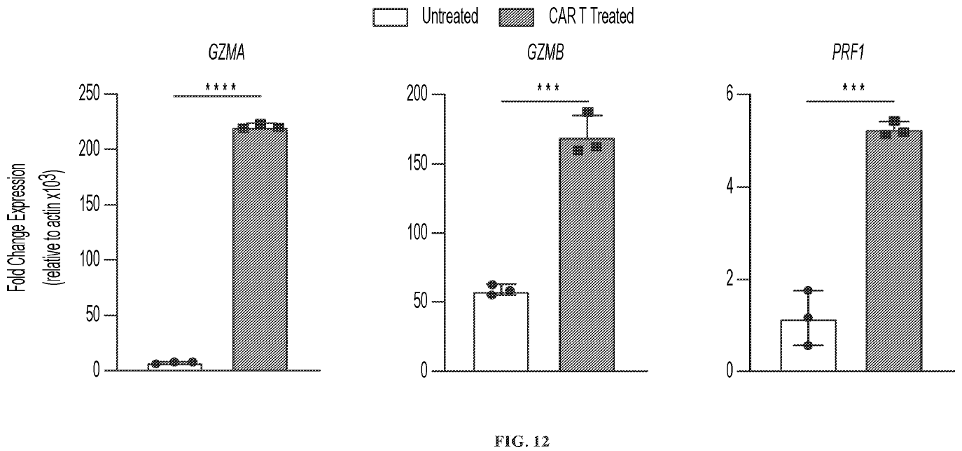

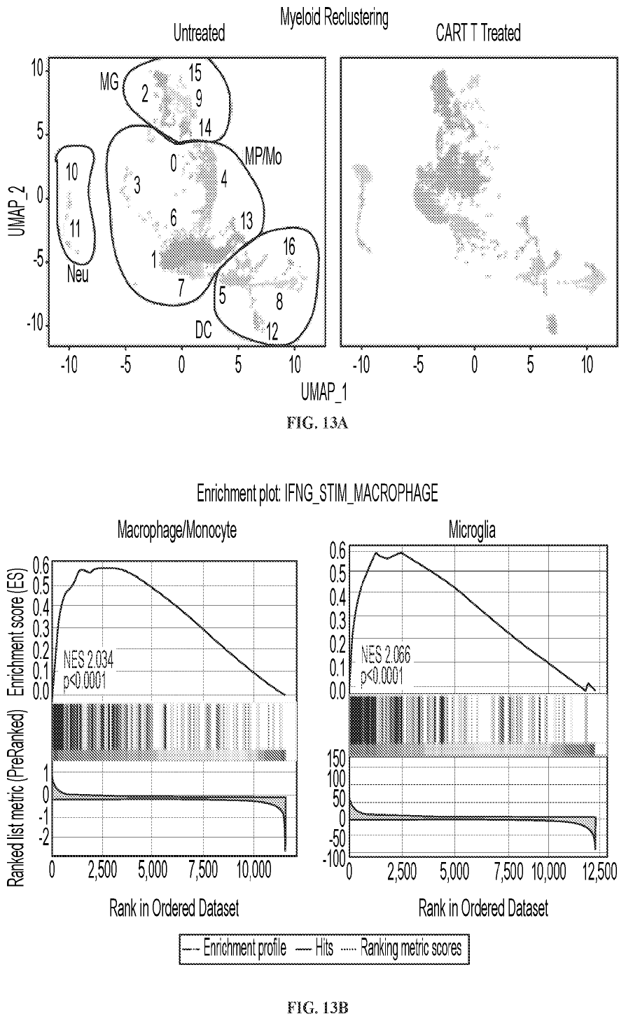

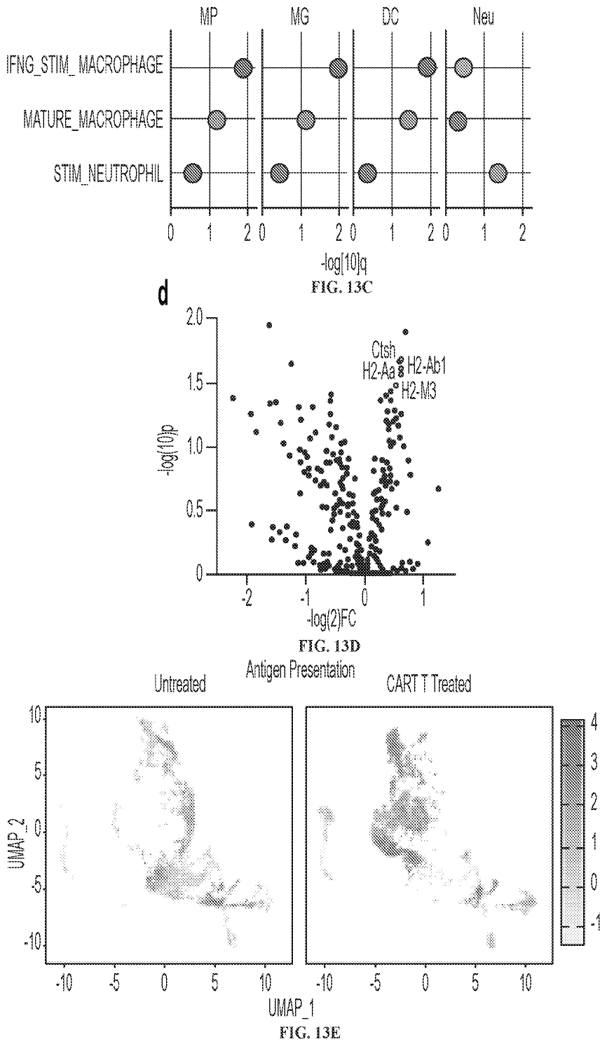

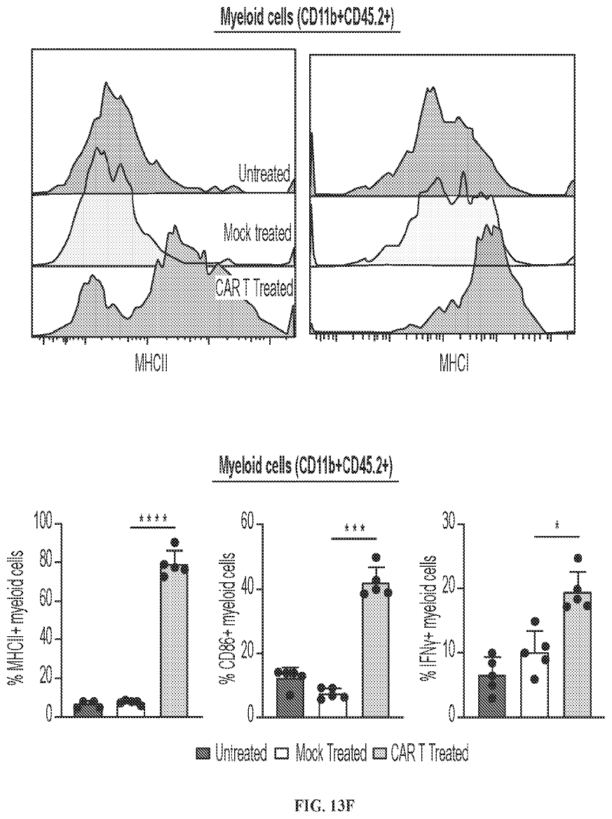

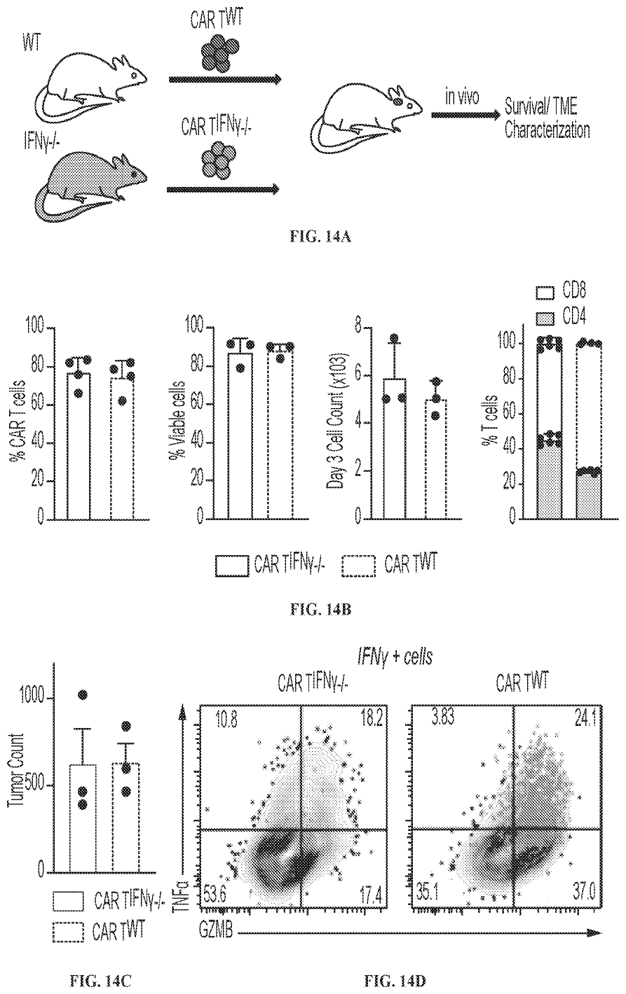

To further characterize T cell populations post-CAR T cell therapy at cellular level and differentiate changes in endogenous versus adoptively transferred T cells, isogenically mismatched CD45.1 CAR T cells were used to treat IL13Rα2+K-Luc tumors engrafted in CD45.2 mice (FIG. 9e). Following intracranial delivery, CAR T cells (CD3+CD45.1+CD19+), but not mock-transduced T cells (CD3+CD45.1+CD19−), displayed a significant increase in T cell count and expression in markers of activation (CD69+), cytotoxic function (GZMB+) and proliferation (Ki67+) (FIG. 9f), establishing that the observed effector activity was CAR-dependent. Interestingly, only after CAR T therapy, a significant increase in the endogenous T cells (CD3+CD45.2+) count with activated (CD69+), proliferative (Ki67+), and cytotoxic phenotype (GZMB+) was observed, which was not detected in untreated or mock treated controls (FIG. 9G). This was in line with qPCR analysis demonstrating upregulation of GZMA, GZMB, and PRF1 genes in intratumoral CD3+ T cells after CAR T therapy (FIG. 12). These results establish that CAR T cells promote endogenous T cell activation and expansion, and are consistent with our previous finding that endogenous intratumoral T cells isolated post CAR T cell therapy have antitumor activity (FIGS. 5D-5F). We next evaluated changes in the innate myeloid cells, such as microglia/macrophages, as they represent a dominant immune population in the glioma tumors and have decisive role in glioma pathogenesis (17). Analysis of intratumoral myeloid population at single cell level identified 17 distinct myeloid subpopulations which underwent a striking remodeling following CAR T therapy (FIG. 13A).

We observed an interesting complexity and dynamics of the intratumoral monocyte/macrophage/microglia/DC compartment in glioma TME. While some macrophage/monocyte subpopulations decreased in frequency, other populations expanded and re-shaped the TME. Seven major monocyte/macrophage (Itgam, Cd68), four microglia (Tmem119 and P2ry12), four DC and two clusters of neutrophils (S100A9) subpopulations were identified. Gene set enrichment analysis (GSEA) revealed enrichment of genes associated with IFNγ-stimulated macrophage and microglia in CAR-treated groups (FIG. 13B). Further assessment of the main myeloid populations (macrophage, microglia, and neutrophils) identified higher expressions of genes associated with mature and IFNγ-activated macrophages as well as stimulated neutrophils (FIG. 13C), further confirming that resident innate immune cells have been exposed to IFNγ-mediated activation.

Nanostring analysis of intratumoral microglia/macrophages cells (CD11b+) from the TME 3 days post-CAR T therapy showed enrichment of genes that mediate antigen processing and presentation (e.g., Cd74, H2-Ab1, H2-Aa, H2-Eb1) (FIG. 13D). Further analyses with scRNAseq revealed that majority of macrophage/microglia subclusters may be involved in antigen processing and presentation (FIG. 13E). Assessing CAR T cell mediated changes in resident microglia/macrophage populations by flow cytometry, we found an increased frequency and number of activated brain-resident macrophage/microglia cells (CD86+, MHCI^{high}, MHCII^{high}) in CAR T-treated mice (FIG. 13F). Assessment of myeloid compartment also revealed a significant increase in the frequency of proinflammatory cytokine IFNγ+(FIG. 13F). Collectively, these data show that CAR T therapy changes the GBM immune landscape and activates the host innate and adaptive immune cells. These results also further reveal a major role for IFNγ in inducing activation of local immune cells.

### Example 4: Lack of IFNγ in CAR T Cells Impairs In Vivo Antitumor Activity and Activation of Host Immune Cells

Given that the myeloid cells constituted the largest population in the glioma TME and our scRNAseq analysis identified gene-signatures related to IFNγ-stimulation within the macrophages and microglia subclusters (FIG. 13B), we reasoned that IFNγ produced by stimulated CAR T cells may play a role in modulating the activation of resident macrophage/microglia cells and subsequent priming and induction of adaptive immune response.

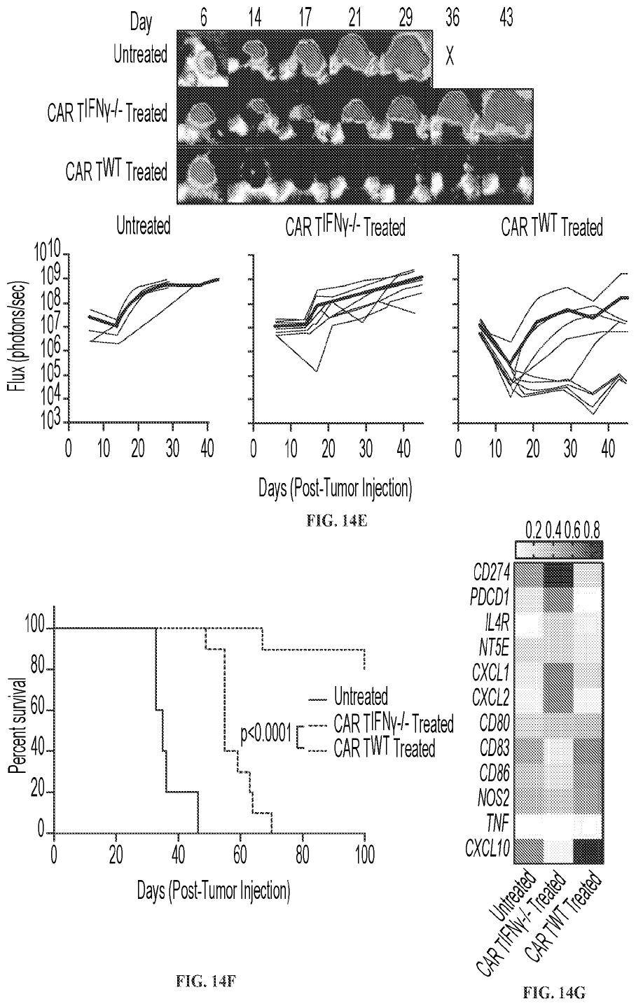

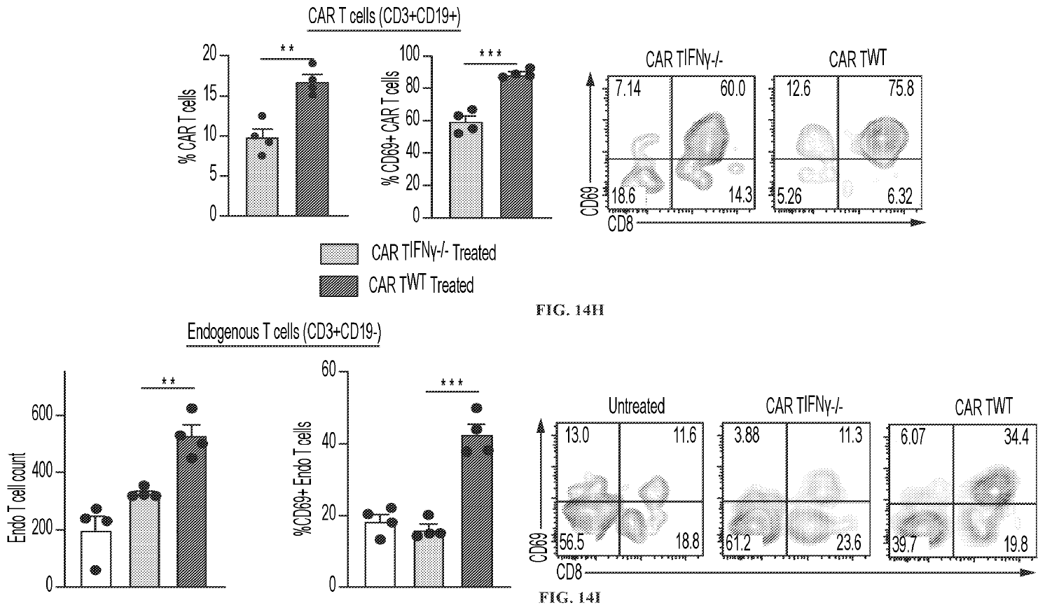

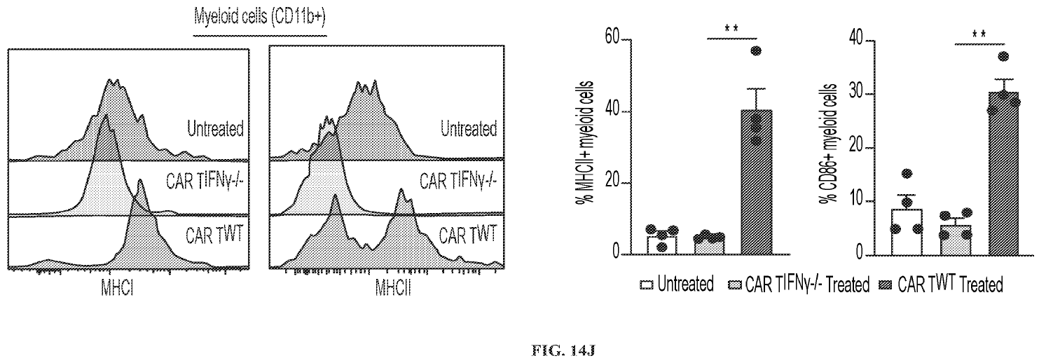

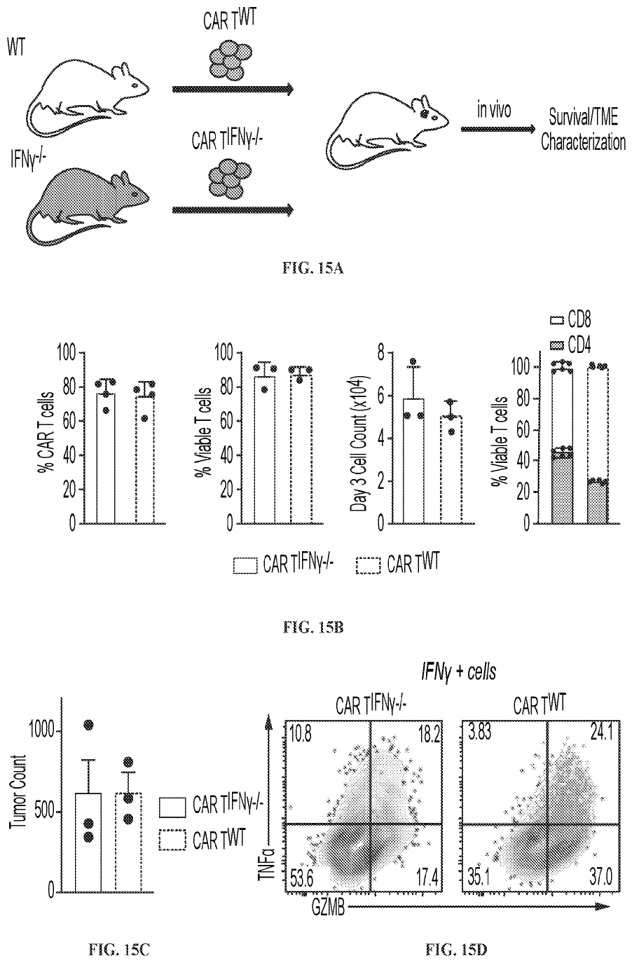

IFNγ is one of the key effector cytokines abundantly produced by CAR T cells upon activation and is a prototypic macrophage activator (18). To investigate whether IFNγ secreted by CAR T cells is responsible for changes observed in phenotype and function of resident macrophages/microglia cells, CAR T cells were developed from WT (CAR T^{wt}) or IFNγ−/− (CAR T^{IFNγ-/-}) mice (FIG. 14A) and characterized accordingly. CAR transduction efficiency, cell viability, expansion and ratio of CD4:CD8 in both CAR T cell populations (CAR T^{wt} and CAR T^{IFNγ-/-}) showed comparable therapeutic products (FIG. 14B). We next verified the functionality of CAR T cells derived from IFNγ−/− mice by conducting an in vitro killing assay in comparison with CAR T cells from WT mice, which demonstrated comparable killing potency at a 1:1 effector to target ratio (FIG. 14C). Assessment of CAR T polyfunctionality demonstrated comparable production of TNFα and GZMB in both CAR T^{wt} and CAR T^{IFNγ-/-} cells with expected lack of IFNγ production in CAR T^{IFNγ-/-} (FIG. 14D). In vivo, mice that received CAR T^{IFNγ-/-} exhibited poor overall survival compared to mice treated with CAR T^{wt}, indicating that IFNγ deficiency in CAR T cells dampens their antitumor activity and results in poor survival (FIG. 14E and FIG. 14F). Analysis of total TME showed enhanced expression in genes involved in activation and proinflammatory cytokines in mice that received CAR T^{wt} and conversely reduced expression of genes involved in suppressive phenotype and function of intratumoral immune infiltrates (FIG. 14G) indicating that lack of IFNγ secretion by CAR T cells changes the glioma TME. IFNγ is a pleiotropic cytokine that induces activation of CD8 T cells (9), promotes polarization of Th1 CD4 cells (19) and reprograms or activates macrophage/microglia cells (6, 7). Therefore, we then assessed whether lack of IFNγ secreted by CAR T cells impacted the host immune cells. Flow cytometry analysis of TME 3 days post CAR T cell therapy revealed a significant decrease in T cell number, both endogenous and CAR T cells, that correlated with a reduction in activated (CD69+) T cells (FIG. 14H and FIG. 14I). Furthermore, a significant increase in frequency of MHCI+/MHCII+ and CD86+ macrophage/microglia cell activation in tumor bearing mice that received CAR T^{wt} compared with CAR T^{IFNγ-/-} cells (FIG. 14J) was observed. Thus, IFNγ production as a consequence of CAR T antitumor activity results in activation and reinvigoration of T cells and increase activation and the antigen presenting potential of macrophage/microglia cells.

### Example 5: Lack of IFNγ-Signaling in the Host Results in Dampen CAR T Antitumor Activity In Vivo

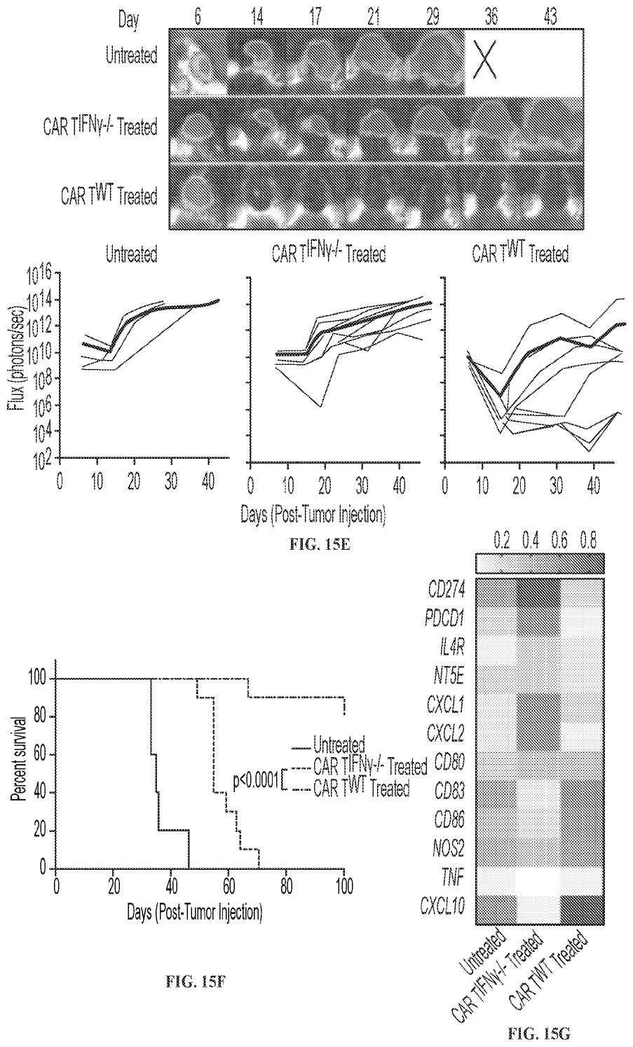

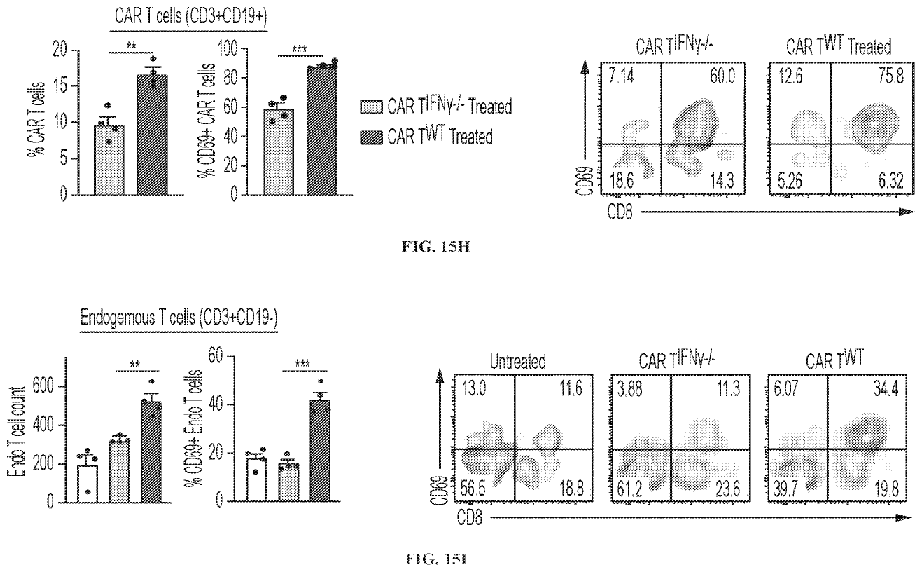

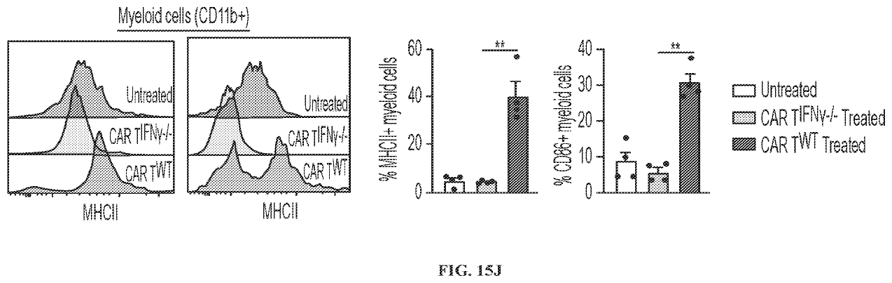

Previous studies have reported IFNγ signaling as a signature of response to immunotherapies such as anti-PD1 treatment (20). In order to investigate whether host IFNγ signaling plays a role in the CAR T-mediated immune response, CAR T^{wt} cells were adoptively transferred into K-Luc-bearing WT or IFNγR−/− mice (FIG. 15A). IFNγR−/− mice that received CAR T^{wt} demonstrated a survival disadvantage, suggesting that lack of IFNγ signaling in the host immune cells dampens the antitumor activity of CAR T cells and the overall survival (FIG. 15B and FIG. 15C). Gene expression analysis of TME revealed that lack of IFNγ signaling in the host during CAR therapy resulted in reduced expression of genes involved in activation and proinflammatory responses (CD40, NOS2, TNFα, GZMA, GZMB, PRF1, and IFNγ) (FIG. 15D). To further investigate CAR T cell-mediated changes in the immune landscape, we adoptively transferred isogenically mismatched (Thy1.1+) CAR T^{wt} cells into K-Luc-bearing WT or IFNγR−/− mice. Flow cytometry analysis of TME revealed significant increase in activation of macrophage/microglia cells (CD11b+CD86+MHCI+MHCII+) in WT mice compared to IFNγR−/− mice as early as 3 days post CAR T cell therapy (FIG. 15E). Compared to WT mice, the number of endogenous T cells (Thy1.2+CD3+), activated with proliferative and effector-cytokine producing capacities was significantly lower in IFNγR−/− mice (FIG. 15F). Interestingly, relative to WT mice, the adoptively transferred CAR T cells (Thy1.1+CD3+) exhibited significantly higher counts, proliferative (Ki67+) and cytotoxic phenotype (IFNγ+ and GZMB+), relative to IFNγR−/− mice (FIG. 15G) suggesting potential IFNγ positive feedback from host immune cells that promote CAR T cell activity in the TME. Collectively, these results confirm that there is an interplay between host and adoptively transferred immune cells and host IFNγ signaling in glioma TME is necessary for mounting a potent immune response during CAR T therapy.

To investigate whether IFNγ secreted by CAR T cells is responsible for changes observed in phenotype and function of resident macrophages/microglia cells, CAR T cells were generated from wild-type (CAR T^{WT}) or IFNγ−/− (CAR T^{IFNγ-/-}) mice (FIG. 15A) and characterized accordingly. CAR transduction efficiency, cell viability, expansion in both CAR T-cell populations (CAR T^{WT} and CAR T^{IFNγ-/-}) showed comparable therapeutic products with some difference in ratio of CD4:CD8 T cells (P<0.05; FIG. 15B). We next verified the functionality of CAR T cells derived from IFNγ−/− mice by conducting an in vitro killing assay in comparison with CAR T cells from WT mice, which demonstrated comparable killing potency at a 1:1 E:T ratio (FIG. 15C). Assessment of CAR T-cell polyfunctionality demonstrated comparable production of TNFα and GZMB in both CAR T^{WT} and CAR T^{IFNγ-/-} cells with expected lack of IFNγ production in CAR T^{IFNγ-/-} (FIG. 15D). In vivo, mice that received CAR T^{IFNγ-/-} exhibited poor overall survival compared with mice treated with CAR T^{TW}, indicating that IFNγ deficiency in CAR T cells dampens their antitumor activity in vivo (FIGS. 15E and 15F). Gene expression analysis of tumor and the associated TME 3 days post-CAR T-cell therapy revealed enhanced expression in genes involved in activation and proinflammatory cytokines in mice that received CAR Tw cells and, conversely, reduced expression of genes involved in suppressive phenotype and function of intratumoral immune infiltrates (FIG. 15G), indicating that lack of IFNγ secretion by CAR T cells changes the glioma TME. IFNγ is a pleiotropic cytokine that induces activation of CD8 T cells, promotes polarization of Th1 CD4 cells, and reprograms or activates macrophage/microglia cells. Therefore, we then assessed whether lack of IFNγ secreted by CAR T cells affected the host immune cells. Flow cytometry analysis of TME 3 days post-CAR T-cell therapy revealed a significant decrease in T-cell number, in both endogenous and CAR T cells, which correlated with a reduction in activated (CD69*) T cells (FIGS. 15H and 15I). Furthermore, a significant increase in frequency of MHCI⁺/MHCII⁺ and CD86⁺ macrophage/microglia cell activation in tumor-bearing mice that received CAR T^{WT} compared with CAR T^{IFNγ-/-} cells (FIG. 15J) was observed. Importantly, lack of IFNγ secretion by the CAR T cells resulted in higher M2-type intratumoral macrophages in mice that received CAR T^{IFNγ-/-} cells compared with CAR T^{WT} cells. Thus, IFNγ production as a consequence of CAR T-cell antitumor activity resulted in activation and reinvigoration of T cells and reprogramming of macrophage/microglia cells to enhance their activation and antigen-presenting potential.

### Example 6: Human CAR T Cell Therapy Modulates Patient Host Immune Cells

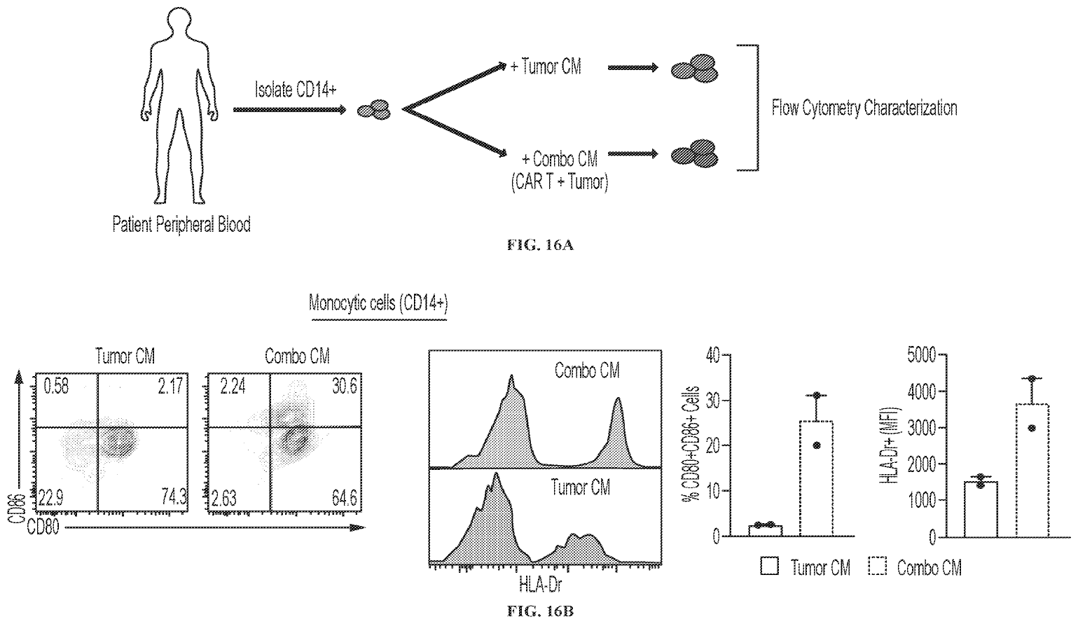

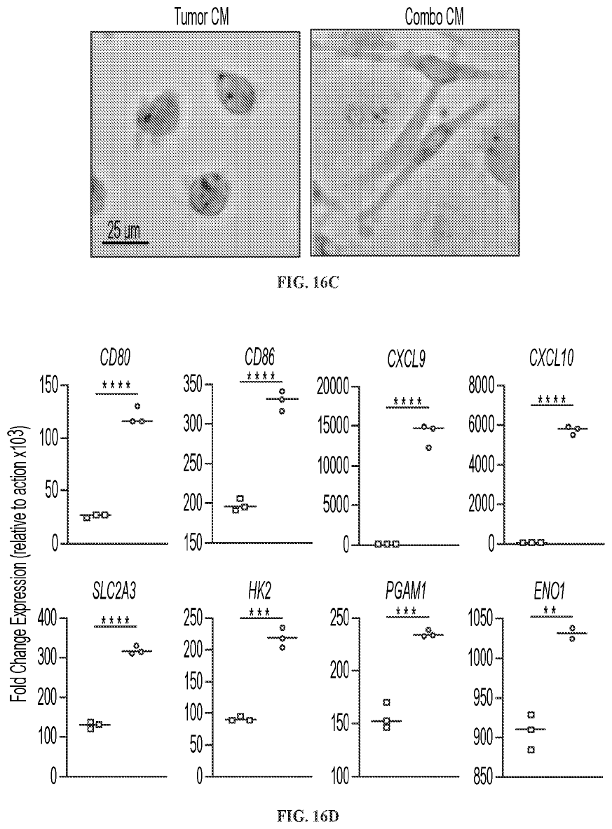

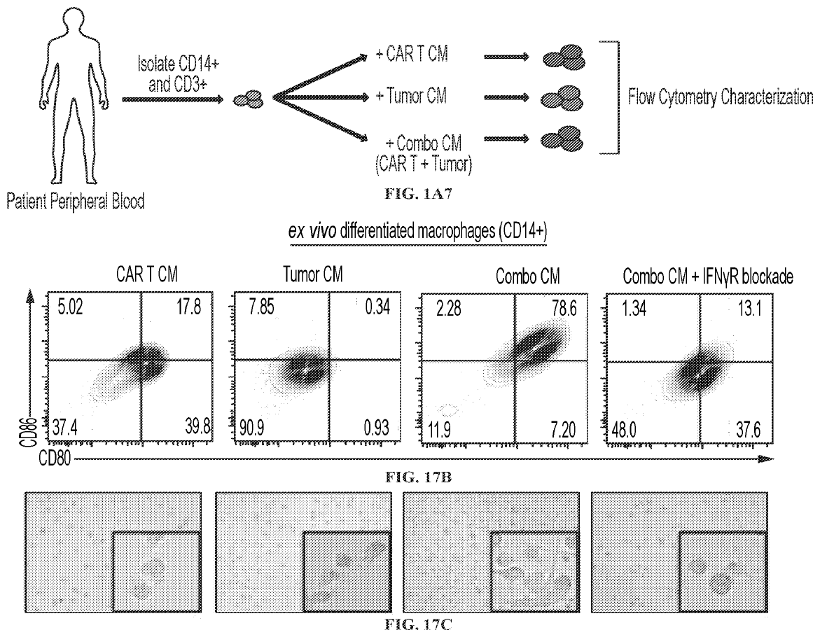

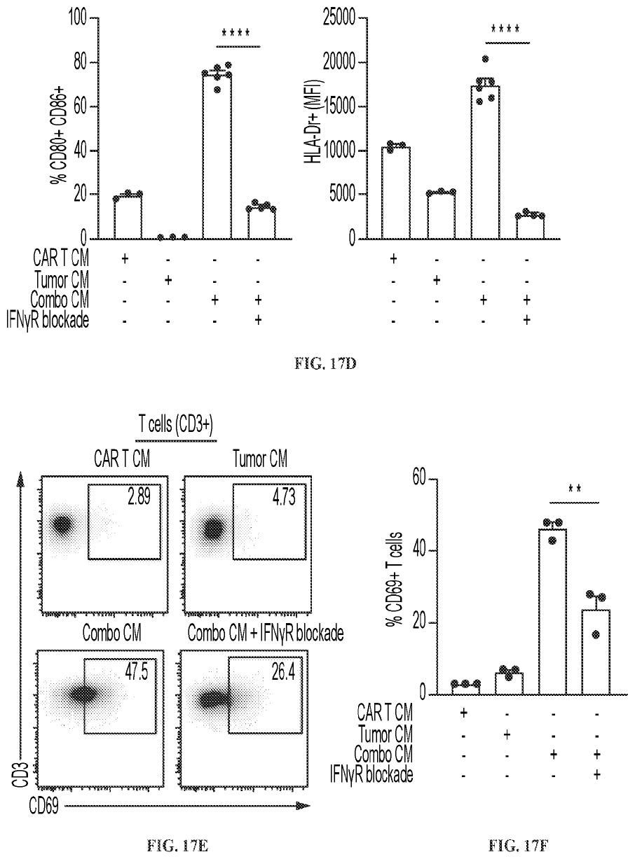

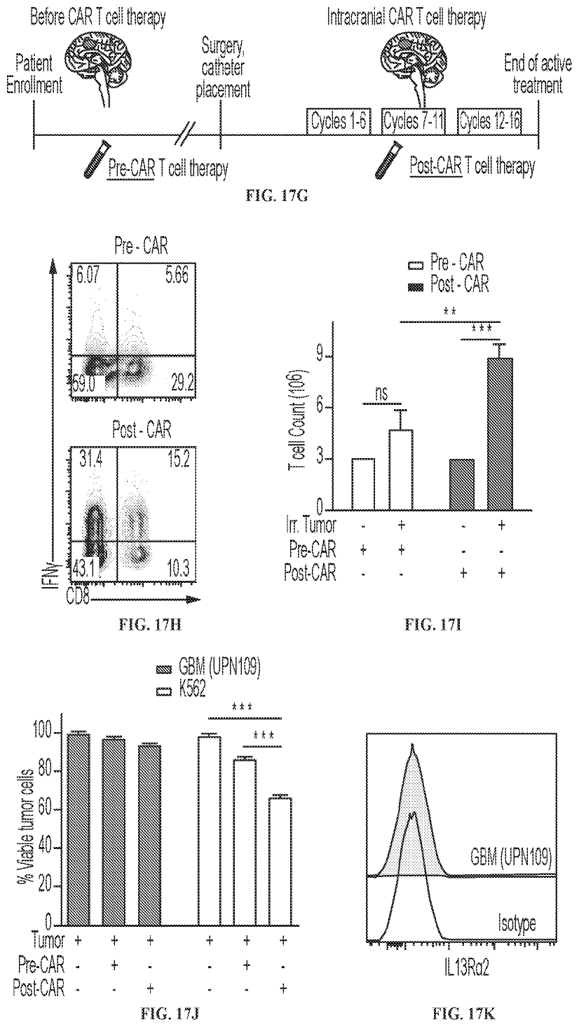

We next investigated the impact of CAR T cell antitumor activity on human endogenous immune cells in GBM patients. To clinically assess if CAR T cells are able to promote activation of GBM patient monocytes or macrophages, we developed an in vitro assay to phenotypically characterize patient myeloid populations in the presence of CAR T cell antitumor activity. Supernatants from co-culture of human CAR T cells against patient-derived glioma tumors were collected and subsequently incubated with glioma patient derived-monocytes (FIG. 16A), ex vivo differentiated patient macrophages or total CD3+ patient T cells (FIG. 17A). Phenotypic and morphological assessments revealed that conditioned media (CM) from CAR T-tumor co-culture promoted differentiation and activation of patient derived-monocytes or macrophages, as demonstrated by increase in expression of activation markers (CD14+CD86+/CD80+ and CD14+HLADr+), which is correlated with enhanced expression of genes associated with classical M1 macrophages (FIGS. 16B-16D and FIGS. 17B-17D). Accordingly, exposure to CM from CAR T-tumor co-culture resulted in induced activation of isolated T cells from GBM patient blood, as evidenced by increased expression of CD69 (FIG. 17E and FIG. 17F). Importantly, blockade of IFNγ signaling in macrophages and T cells resulted in reduced activation (FIG. 17B-17F), highlighting the impact of IFNγ in the CAR T-mediated activation of host immune cell in GBM patients. Taken together these findings suggest that IFNγ secreted by CAR T cell antitumor function promotes polarization of monocytes into activated macrophages, further induce activation of differentiated macrophages and promote T cell activation.

Lastly, we aimed to assess if CAR T cells have the potential to induce generation of tumor-specific T cells in clinical setting, as we demonstrated in our immune competent mouse models of GBM. In order to precisely investigate this phenomenon, we evaluated samples from a case report that exhibited a complete response and was a unique responder to CAR T therapy (4). T cells were isolated from blood before CAR T cell treatment (Pre-CAR T) and during response to CAR T therapy (Post-CAR T) (FIG. 17G). Isolated T cells were stimulated and expanded in the presence of irradiated autologous tumor cells. Flow cytometry assessment of T cell populations revealed increased tumor reactivity as indicated by increased intracellular IFNγ and proliferation for T cells isolated during response versus prior to the initiation of CAR T cell therapy (FIG. 17H and FIG. 17I). Importantly, in a patient derived co-culture of ex vivo expanded T cells with autologous patient glioma tumor or irrelevant tumor cell line, T cells isolated during response to CAR T therapy exhibited tumor-specific killing against autologous versus irrelevant tumor cells (FIG. 17J). These results were in light of the tumor cells being IL13Rα2 negative (FIG. 17K). These findings confirm our preclinical studies that an effective CAR T therapy has the potential to stimulate the host immune cells, promote generation of tumor-specific T cell responses and furthermore, the host immune cells play an important role in a successful CAR T treatment.

### Example 7: Co-Expression of IL-13 CAR T and Interferon Gamma

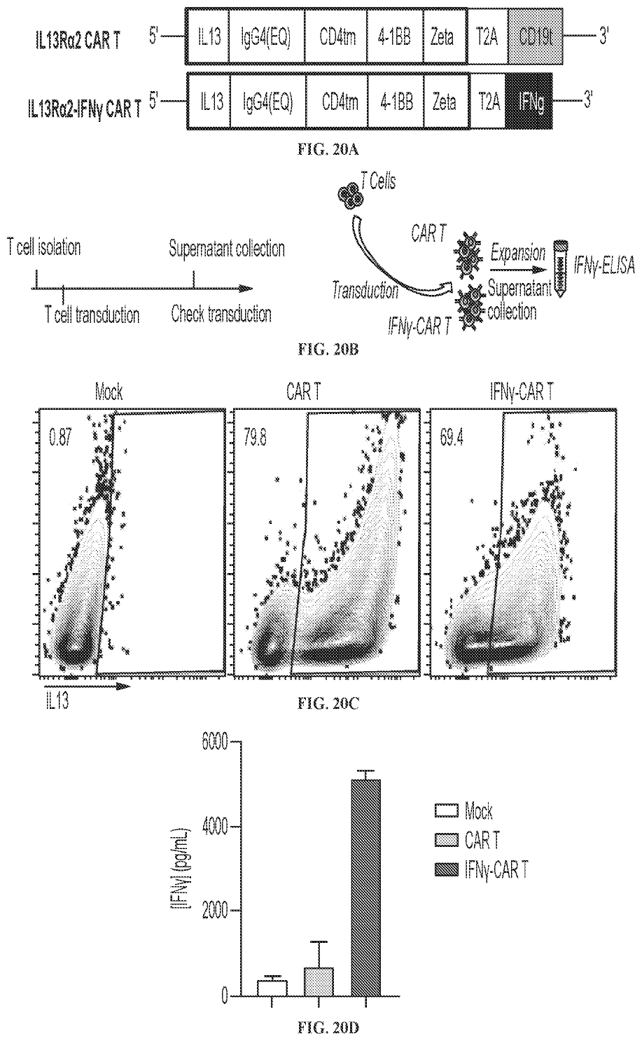

We examined the impact of co-expressing interferon gamma by creating an expression cassette in which the IL-13 CAR of Example 1 (FIG. 1A) is joined to immature murine interferon gamma via a T2A skip sequence (FIG. 20A). We designed and constructed IL13Rα2-CAR/IFNγ for murine and human platforms and demonstrate that incorporating IFNγ in the CAR cassette is feasible with comparable transduction and expansion of CAR T cells (FIG. 20A). The vectors were sequenced and verified. Murine T cells were isolated transduced with either a vector expressing the IL-13 CAR and truncated CD19 (lacking a signaling domain) or the IL-13 CAR and murine interferon gamma (FIG. 20B). Culture supernatant was collected and flow cytometry was used to assess the presence of IL-13 as a marker for CAR expression. Both constructs expressed the IL-13 CAR, and transduction efficiency is above 50% by FACS (FIG. 20C). Furthermore, to verify that IL13Rα2-CAR/IFNγ vectors endow T cells with the ability to express and secrete IFNγ, we collected supernatant from ex vivo expanded IL13Rα2-CAR/IFNγ and IL13Rα2-CAR T cells and measured IFNγ levels with ELISA (FIG. 1D). Measurement of interferon gamma production by ELISA showed only the IL-13 CAR T-interferon gamma construct expressed interferon gamma (FIG. 20D).

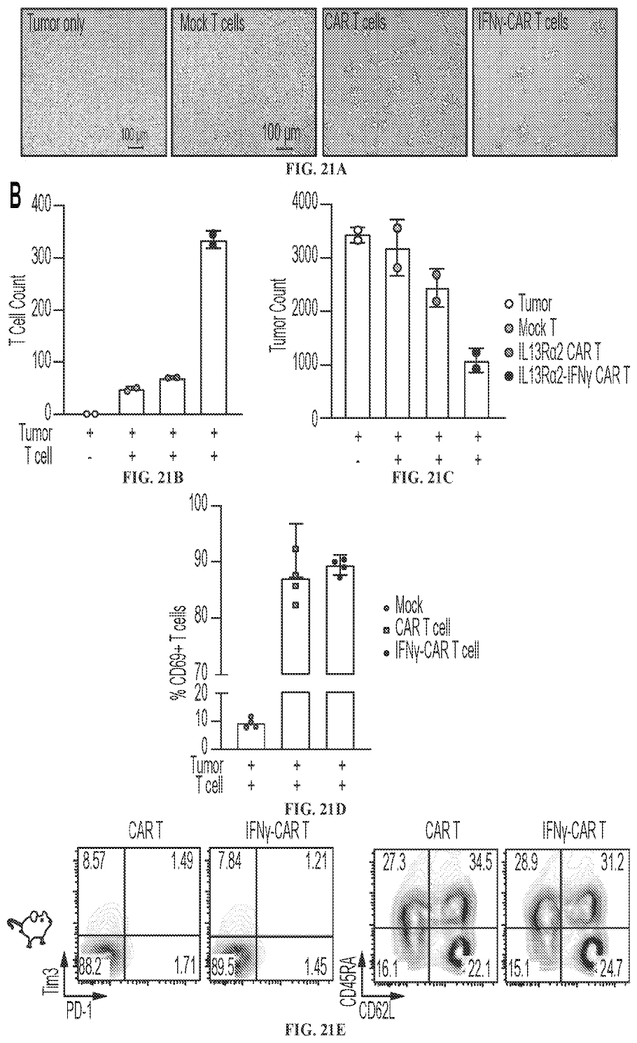

Next, we assessed the phenotype of the CAR T cells. Our studies demonstrated no phenotypic differences between IL13Rα2-CAR/IFNγ and IL13Rα2-CAR in murine (FIG. 21E) and human (FIG. 22C) T cells. Briefly, murine IL-13 CAR T cells and murine IL-13 CAR-interferon gamma T cells were co-cultured with murine glioma tumor cells at a 1:3 effector:target ratio for 24 hours. As demonstrated in FIGS. 21A-21C, the IL-13 CAR-interferon gamma T cells exhibited both superior proliferation and tumor cell killing. T cell activation was assessed by measuring CD69 expression. As can be seen in FIG. 21D, the activation of IL-13 CAR T cells and the IL-13 CAR T-interferon gamma T cells was similar.

CAR T cells expressing an human IL-13 CAR (human IL-13 with E13Y mutation, human CD8 hinge, human CD8 TM, human 4-1BB co-stimulatory domain and human CD3 zeta) with our without co-expressed human interferon gamma were produced. The human IL-13 CAR T cells and human IL-13 CAR-interferon gamma T cells were co-cultured with patient-derived glioma tumor cells at a 1:25 effector:target ratio for 24 hours. T cells and tumor cells were assessed. As can be seen in FIG. 22A, the IL-13 CAR-interferon gamma T cells exhibited superior proliferation and roughly similar T cell killing. Importantly, when co-cultured with antigen positive tumors, there is increased expansion of IL13Rα2-CAR/IFNγ T cells compared to IL13Rα2-CAR T cells in murine (FIG. 21C) and human (FIG. 22B). In both murine and human, a slightly enhanced killing capacity is observed in IL13Rα2-CAR/IFNγ T cells compared to IL13Rα2-IFNγ CAR T cells.

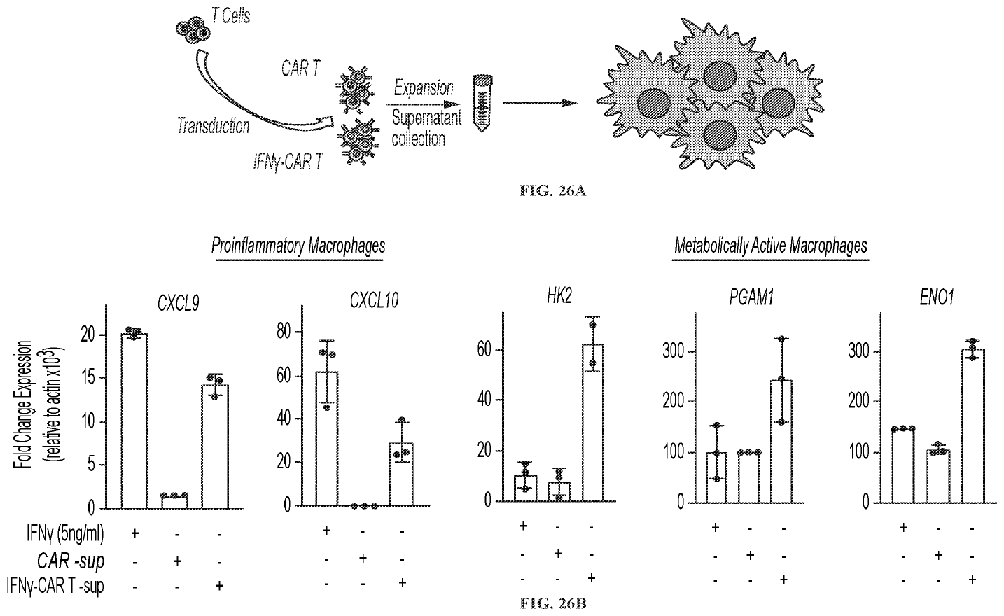

Importantly, to verify that the secreted IFNγ from IL13Rα2-CAR/IFNγ T cells were functional and have immune stimulatory components, supernatant from ex vivo expanded T cells engineered to constitutively express IFNγ were added to macrophage cultures (FIG. 26A). Macrophages exposed to IL13Rα2-CAR/IFNγ culture conditions exhibited greater proinflammatory and metabolically active phenotype as compared to supernatants collected from IL13Rα2-CAR T cells (FIG. 26B). Collectively, these results show that IL13Rα2-CAR/IFNγ express and secrete IFNγ and have the potential to activate endogenous innate immune populations such as macrophages.

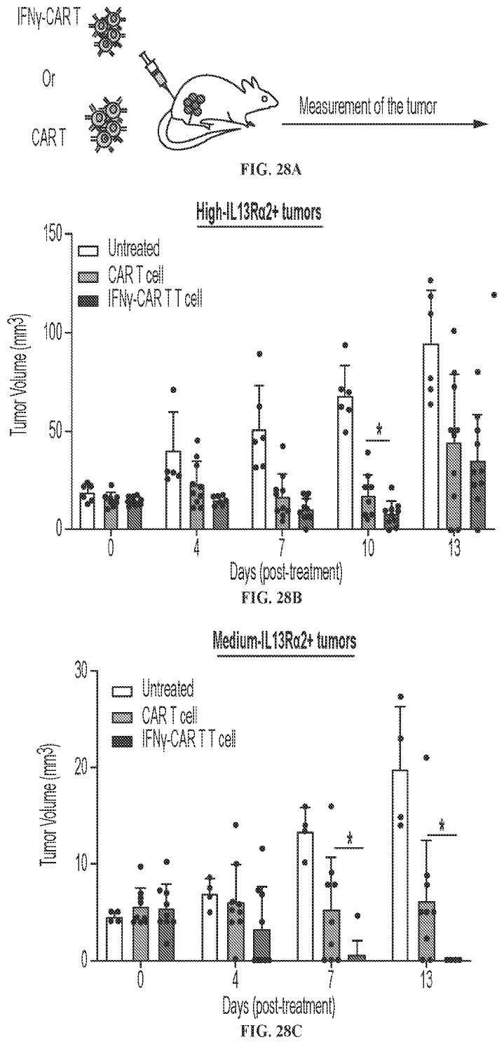

We have conducted a pilot study assessing the antitumor activity of IL13Rα2-CAR/IFNγ vs. IL13Rα2-CAR in mice bearing tumors with high (FIG. 28B; left) or medium/low (FIG. 28C; right) IL13Rα2 antigen in our syngeneic model. While the function of the two CARs are comparable in high antigen expressing tumors, there is a significant change in the antitumor activity in mice bearing medium antigen expressing tumors treated with IL13Rα2-CAR/IFNγ compared to IL13Rα2-CAR.

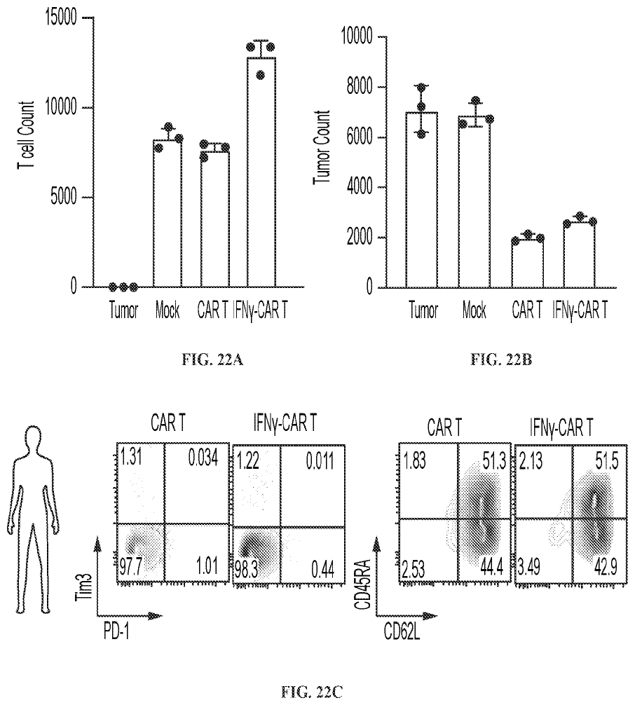

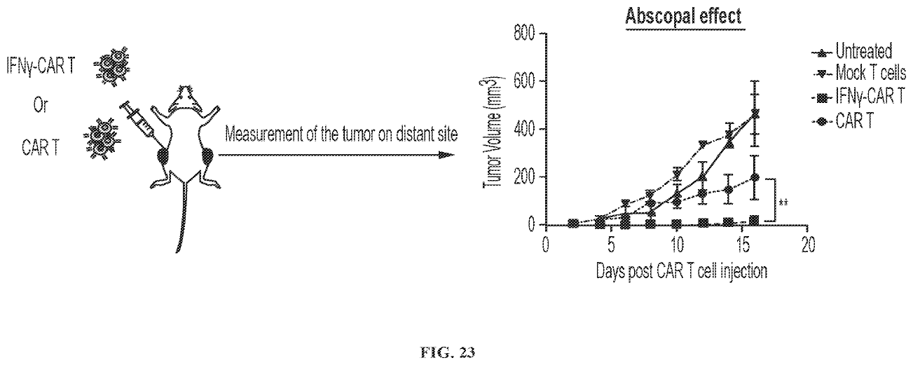

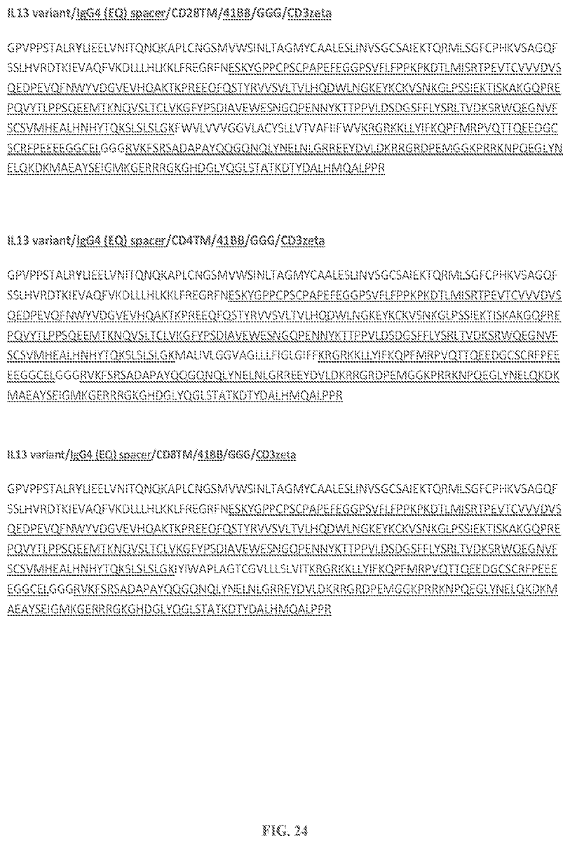

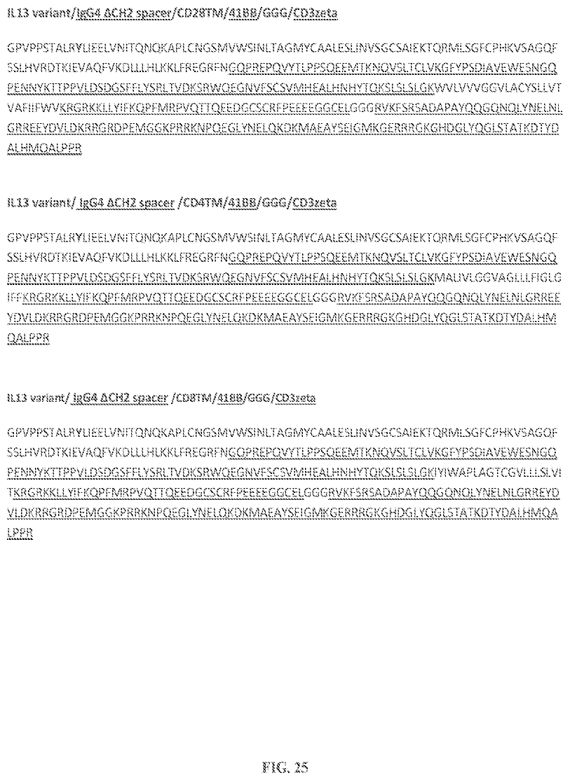

As part the global impact of IFNγ, we also tested whether IL13Rα2-IFNγ CARs exhibit superior antitumor activity against metastatic diseases or tumors at distant sites. Thus, murine IL-13 CAR T cells and murine IL-13 CAR-interferon gamma T cells were assessed in a murine model of metastatic melanoma (FIG. 23). Briefly, tumor cells were injected into both flanks of mice. Once the tumors reached a predetermined size, Murine IL-13 CAR T cells and murine IL-13 CAR-interferon gamma T cells were injected locally to one tumor. Tumor size was measured on both sides. The graph in FIG. 23 shows the tumor volume on the non-treated side. As can be seen, the IL-13 CAR T-interferon gamma T cells exhibited a greater abscopal effect than the IL-13 CAR T cells. Interestingly, our studies on melanoma bearing mice demonstrate that IL13Rα2-CAR/IFNγ T cells have superior capacity to target distant tumors. These findings strongly highlight IL3Rα2-CAR/IFNγ as an important therapeutic approach for cancer with multifocal lesions such as GBM.

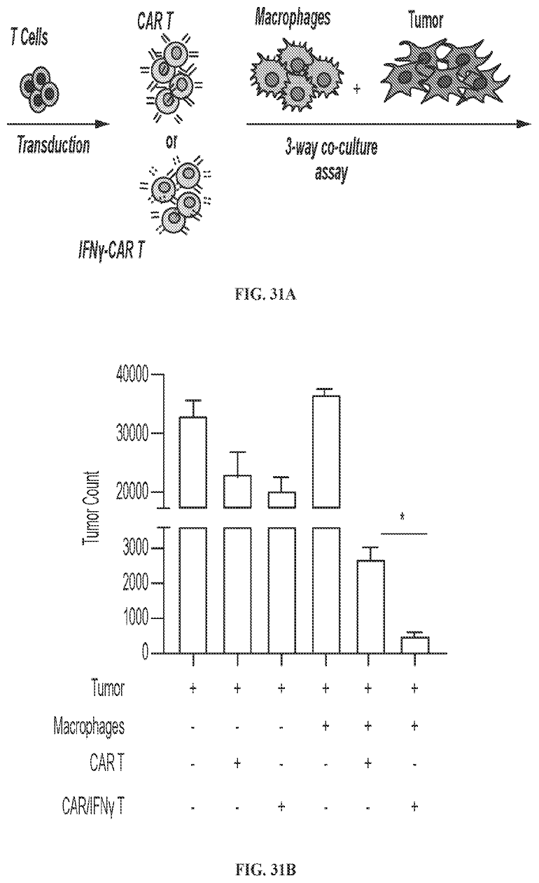

Experiments were designed to test in vivo functional activity of IL13Rα2-IFNγ CAR in syngeneic immunocompetent glioma models and NSG mice implanted with IL13Rα2+ primary brain tumor lines (PBTs). We demonstrated that IL13Rα2-CAR/IFNγ T cells have superior antitumor activity in mice bearing medium/low antigen tumors and eradicate tumors at distant sites in metastatic melanoma model. To assess the importance of IFNγ as immunostimulatory agent and therapeutic importance of IL13Rα2-CAR/IFNγ T cells, we developed a 3-way coculture system using CAR T cells, macrophages, and tumor cells (FIGS. 31A-31B). Our results suggests that in the presence of other immune cells such as macrophages, IL3Rα2-CAR/IFNγ T cells exhibit a significantly superior antitumor function and stimulates endogenous immunity.

### Example 8: Optimizing Co-Expression of IL-13 CAR T and Interferon Gamma

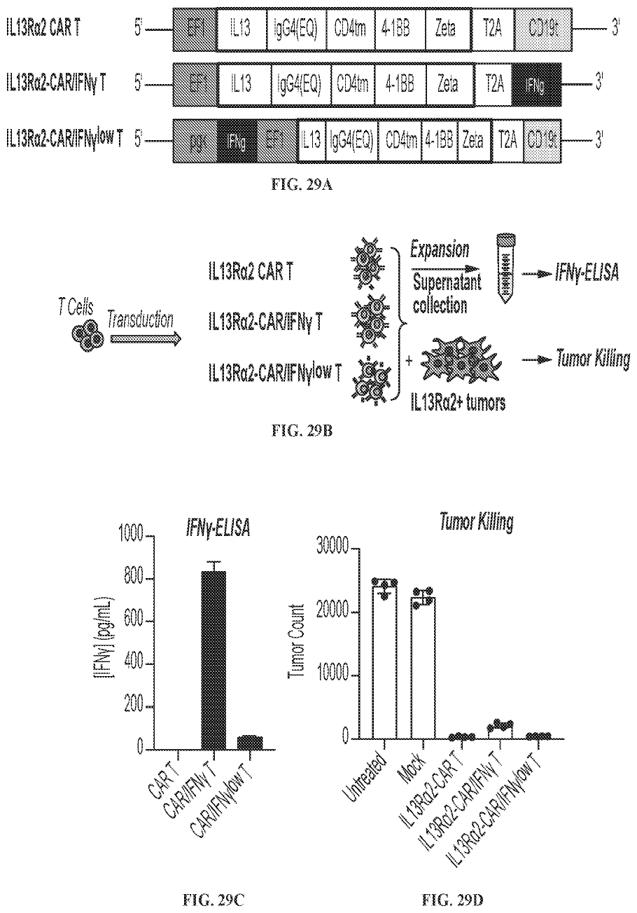

We also designed and constructed different IL13Rα2-CAR/IFNγ variants to prioritize for both efficacy and safety by optimizing and regulating IFNγ expression. We successfully designed and sequence checked the constructs shown in FIG. 29A. T cells were transduced with CAR/IFNγ variants, and supernatant was collected to validate IFNγ production and cocultured with IL13Rα2+ tumors to confirm functionality (FIG. 29B). The CAR/IFNγ variants were assessed for different levels of IFNγ expression and secretion. Relative to EF1 promoter, which is a strong promoter, the pkg100 promoter is a weaker promoter. CAR T cells having IFNγ under the pkg100 promoter showed reduced level of IFNγ (FIG. 29C). The IL13Rα2-CAR/IFNγ [symbol] ^{low} T cell addresses safety concerns related to excessive production of IFNγ. Next, to confirm cytotoxic function, IL13Rα2-CAR/IFN [symbol] ^{low} CAR T cells were cocultured with IL13Rα2+ tumors at 1:50 ratio effector to target for 5 days. Assessment of viable tumor count showed that IL13Rα2-CAR/IFNγ [symbol] ^{low} T cells exhibited comparable cytotoxic function to standard IL13Rα2-CAR T cells (FIG. 29D).

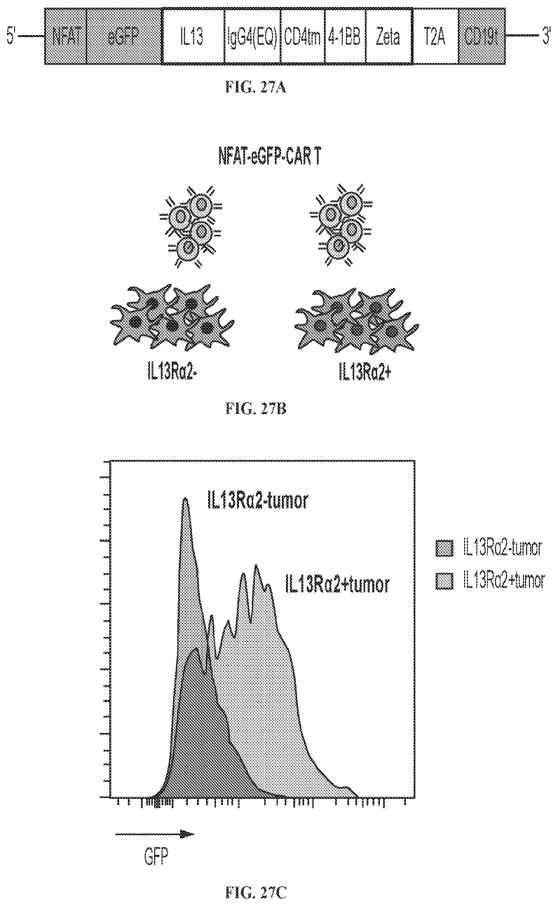

We designed an inducible construct system using a synthetic NFAT promoter to control IFNγ expression. This construct was designed to control the expression of the gene of interest, ensuring that expression of IFNγ will only occur when CAR T cells are activated. As proof of concept, we placed GFP under the control of an NFAT promoter. Our studies demonstrated that upon CAR activation in the presence of IL13Rα2 antigen positive tumors, the NFAT promoter is functional and can induce GFP expression (FIGS. 27A-27C).

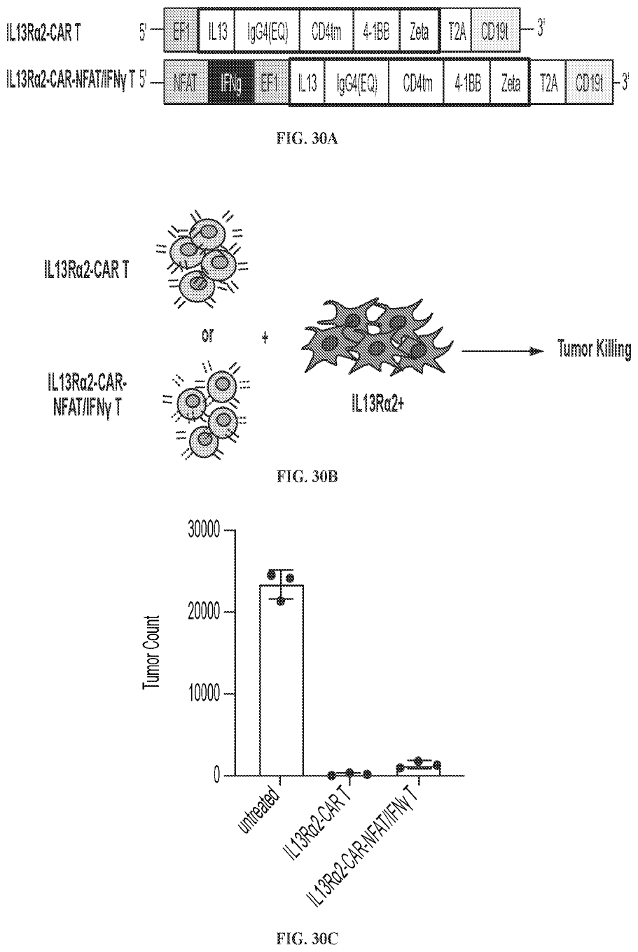

Next, we replaced the GFP gene with IFNγ (FIG. 30A). T cells transduced with the constructs were cocultured with IL13Rα2+ tumors at 1:50 effector to target ratio for 5 days (FIG. 30B). Our results confirms that NFAT CARs are functional and exhibit comparable killing capacity to standard IL13Rα2-CAR T cells (FIG. 30C). These results indicated the incorporation of synthetic NFAT promoter in the CAR construct did not impair the function of the CAR T cells.

### REFERENCES

1. McGranahan T, Therkelsen K E, Ahmad S, Nagpal S. Current State of Immunotherapy for Treatment of Glioblastoma. Current treatment options in oncology 2019, 20(3): 24.
2. Siegel R L, Miller K D, Jemal A. Cancer statistics, 2018. CA: a cancer journal for clinicians 2018, 68(1): 7-30.
3. Alexander B M, Cloughesy T F. Adult Glioblastoma. Journal of clinical oncology:official journal of the American Society of Clinical Oncology 2017, 35(21): 2402-2409.
4. Brown C E, Alizadeh D, Starr R, Weng L, Wagner J R, Naranjo A, et al. Regression of Glioblastoma after Chimeric Antigen Receptor T-Cell Therapy. The New England journal of medicine 2016, 375(26): 2561-2569.
5. Hegde M, Joseph S K, Pashankar F, DeRenzo C, Sanber K, Navai S, et al. Tumor response and endogenous immune reactivity after administration of HER2 CAR T cells in a child with metastatic rhabdomyosarcoma. Nature communications 2020, 11(1): 3549.
6. Duluc D, Corvaisier M, Blanchard S, Catala L, Descamps P, Gamelin E, et al. Interferon-gamma reverses the immunosuppressive and protumoral properties and prevents the generation of human tumor-associated macrophages. International journal of cancer 2009, 125(2): 367-373.
7. Rock R B, Hu S, Deshpande A, Munir S, May B J, Baker C A, et al. Transcriptional response of human microglial cells to interferon-gamma. Genes and immunity 2005, 6(8): 712-719.
8. Castro F, Cardoso A P, Goncalves R M, Serre K, Oliveira M J. Interferon-Gamma at the Crossroads of Tumor Immune Surveillance or Evasion. Frontiers in immunology 2018, 9: 847.
9. Bhat P, Leggatt G, Waterhouse N, Frazer I H. Interferon-gamma derived from cytotoxic lymphocytes directly enhances their motility and cytotoxicity. Cell death & disease 2017, 8(6): e2836.
10. Dunn G P, Koebel C M, Schreiber R D. Interferons, immunity and cancer immunoediting. Nature reviews Immunology 2006, 6(11): 836-848.
11. Curtsinger J M, Mescher M F. Inflammatory cytokines as a third signal for T cell activation. Current opinion in immunology 2010, 22(3): 333-340.
12. Brown C E, Aguilar B, Starr R, Yang X, Chang W C, Weng L, et al. Optimization of IL13Ralpha2-Targeted Chimeric Antigen Receptor T Cells for Improved Anti-tumor Efficacy against Glioblastoma. Molecular therapy: the journal of the American Society of Gene Therapy 2018, 26(1): 31-44.
13. Reilly K M, Loisel D A, Bronson R T, McLaughlin M E, Jacks T. Nf1; Trp53 mutant mice develop glioblastoma with evidence of strain-specific effects. Nature genetics 2000, 26(1): 109-113.
14. Flores C T, Wildes T J, Drake J A, Moore G L, Dean B D, Abraham R S, et al. Lin(−)CCR2(+) hematopoietic stem and progenitor cells overcome resistance to PD-1 blockade. Nature communications 2018, 9(1): 4313.
15. Ausman J I, Shapiro W R, Rail D P. Studies on the chemotherapy of experimental brain tumors: development of an experimental model. Cancer research 1970, 30(9): 2394-2400.
16. Reardon D A, Gokhale P C, Klein S R, Ligon K L, Rodig S J, Ramkissoon S H, et al. Glioblastoma Eradication Following Immune Checkpoint Blockade in an Orthotopic, Immunocompetent Model. Cancer immunology research 2016, 4(2): 124-135.
17. Gutmann D H, Kettenmann H. Microglia/Brain Macrophages as Central Drivers of Brain Tumor Pathobiology. Neuron 2019, 104(3): 442-449.
18. Barton E S, White D W, Cathelyn J S, Brett-McClellan K A, Engle M, Diamond M S, et al. Herpesvirus latency confers symbiotic protection from bacterial infection. Nature 2007, 447(7142): 326-329.
19. Schulz E G, Mariani L, Radbruch A, Hofer T. Sequential polarization and imprinting of type 1 T helper lymphocytes by interferon-gamma and interleukin-12. Immunity 2009, 30(5): 673-683.
20. Cloughesy T F, Mochizuki A Y, Orpilla J R, Hugo W, Lee A H, Davidson T B, et al. Neoadjuvant anti-PD-1 immunotherapy promotes a survival benefit with intratumoral and systemic immune responses in recurrent glioblastoma. Nature medicine 2019, 25(3): 477-486.
21. Maus M V, Fraietta J A, Levine B L, Kalos M, Zhao Y, June C H. Adoptive immunotherapy for cancer or viruses. Annual review of immunology 2014, 32: 189-225.
22. Quail D F, Joyce J A. The Microenvironmental Landscape of Brain Tumors. Cancer cell 2017, 31(3): 326-341.
23. Gieryng A, Pszczolkowska D, Walentynowicz K A, Rajan W D, Kaminska B. Immune microenvironment of gliomas. Laboratory investigation; a journal of technical methods and pathology 2017, 97(5): 498-518.
24. Jansen C S, Prokhnevska N, Master V A, Sanda M G, Carlisle J W, Bilen M A, et al. An intra-tumoral niche maintains and differentiates stem-like CD8 T cells. Nature 2019, 576(7787): 465-470.
25. Corrales L, Glickman L H, McWhirter S M, Kanne D B, Sivick K E, Katibah G E, et al. Direct Activation of STING in the Tumor Microenvironment Leads to Potent and Systemic Tumor Regression and Immunity. Cell reports 2015, 11(7): 1018-1030.
26. Corrales L, McWhirter S M, Dubensky T W, Jr., Gajewski T F. The host STING pathway at the interface of cancer and immunity. The Journal of clinical investigation 2016, 126(7): 2404-2411.
27. Kaufmann E, Sanz J, Dunn J L, Khan N, Mendonca L E, Pacis A, et al. BCG Educates Hematopoietic Stem Cells to Generate Protective Innate Immunity against Tuberculosis. Cell 2018, 172(1-2): 176-190 el 19.
28. Yao Y, Jeyanathan M, Haddadi S. Barra N G, Vaseghi-Shanjani M, Damjanovic D, et al. Induction of Autonomous Memory Alveolar Macrophages Requires T Cell Help and Is Critical to Trained Immunity. Cell 2018, 175(6): 1634-1650 e1617.
29. Grasso C S, Tsoi J, Onyshchenko M, Abril-Rodriguez G, Ross-Macdonald P. Wind-Rotolo M. et al. Conserved Interferon-gamma Signaling Drives Clinical Response to Immune Checkpoint Blockade Therapy in Melanoma. Cancer cell 2020.
30. O'Rourke D M, Nasrallah M P, Desai A, Melenhorst J J, Mansfield K, Morrissette J J D, et al. A single dose of peripherally infused EGFRvII-directed CAR T cells mediates antigen loss and induces adaptive resistance in patients with recurrent glioblastoma. Sci Transl Med 2017, 9(399).
31. You F, Jiang L, Zhang B, Lu Q, Zhou Q, Liao X, et al. Phase 1 clinical trial demonstrated that MUC1 positive metastatic seminal vesicle cancer can be effectively eradicated by modified Anti-MUC1 chimeric antigen receptor transduced T cells. Science China Life sciences 2016, 59(4): 386-397.
32. Wang D, Aguilar B, Starr R, Alizadeh D, Brito A, Sarkissian A, et al. Glioblastoma-targeted CD4+ CAR T cells mediate superior antitumor activity. JCI insight 2018, 3(10): e99048.
33. Brown C E, Alizadeh D, Starr R, Weng L, Wagner J R, Naranjo A, et al. Regression of Glioblastoma after Chimeric Antigen Receptor T-Cell Therapy. The New England journal of medicine 2016, 375(26): 2561-2569.
34. Wang D, Starr R, Chang W C, Aguilar B, Alizadeh D, Wright S L, et al. Chlorotoxin-directed CAR T cells for specific and effective targeting of glioblastoma. Sci Transl Med 2020, 12(533).
35. Wang D, Aguilar B, Starr R, Alizadeh D, Brito A, Sarkissian A, et al. Glioblastoma-targeted CD4+ CAR T cells mediate superior antitumor activity. JCI insight 2018, 3(10).
36. Brown C E, Starr R, Martinez C, Aguilar B, D'Apuzzo M, Todorov I, et al. Recognition and killing of brain tumor stem-like initiating cells by CD8+ cytolytic T cells. Cancer research 2009, 69(23): 8886-8893.
37. Dudley M E, Rosenberg S A. Adoptive-cell-transfer therapy for the treatment of patients with cancer. Nature reviews Cancer 2003, 3(9): 666-675.
38. Alizadeh D, White E E, Sanchez T C, Liu S, Zhang L, Badie B, et al. Immunostimulatory CpG on Carbon Nanotubes Selectively Inhibits Migration of Brain Tumor Cells. Bioconjugate chemistry 2018, 29(5): 1659-1668.
39. Stuart T, Butler A, Hoffman P, Hafemeister C, Papalexi E, Mauck W M, 3rd, et al. Comprehensive Integration of Single-Cell Data. Cell 2019, 177(7): 1888-1902 e1821.
40. Heng T S, Painter M W, Immunological Genome Project C. The Immunological Genome Project: networks of gene expression in immune cells. Nature immunology 2008, 9(10): 1091-1094.
41. Aran D, Looney A P, Liu L, Wu E, Fong V, Hsu A, et al. Reference-based analysis of lung single-cell sequencing reveals a transitional profibrotic macrophage. Nature immunology 2019, 20(2): 163-172.
42. Godec J, Tan Y, Liberzon A, Tamayo P, Bhattacharya S, Butte A J, et al. Compendium of Immune Signatures Identifies Conserved and Species-Specific Biology in Response to Inflammation. Immunity 2016, 44(1): 194-206.
43. Yu G, Wang L G, Han Y, He Q Y. clusterProfiler: an R package for comparing biological themes among gene clusters. Omics: a journal of integrative biology 2012, 16(5): 284-287.

### Other Embodiments

It is to be understood that while the invention has been described in conjunction with the detailed description thereof, the foregoing description is intended to illustrate and not limit the scope of the invention.

What is claimed is:

1. A nucleic acid molecule comprising a nucleotide sequence encoding a chimeric antigen receptor (CAR), wherein the chimeric antigen receptor comprises: a targeting domain, a spacer, a transmembrane domain, a co-stimulatory domain, and a CD3ζ signaling domain; and a nucleotide sequence encoding a polypeptide comprising a human interferon gamma or a variant thereof.

2. The nucleic acid molecule of claim 1, wherein the nucleic acid molecule comprises a promoter that controls expression of both the CAR and human interferon gamma.

3. The nucleic acid molecule of claim 1, wherein a first promoter controls expression of the CAR and a second promoter controls expression of the human interferon gamma or variant thereof.

4. The nucleic acid molecule of claim 3, wherein the first promoter is a constitutive promoter and the second promoter is a constitutive promoter or is an inducible promoter.

5. The nucleic acid molecule of claim 1, wherein a nucleotide sequence encoding a 2A skip sequence is located between the nucleotide sequence encoding a CAR and the nucleotide sequence encoding a human interferon gamma or a variant thereof.

6. The nucleic acid molecule of claim 5, wherein the 2A skip sequence is selected from the group consisting of T2A, P2A, E2A and F2A.

7. The nucleic acid molecule of claim 1, wherein the human interferon gamma or variant thereof comprises the amino acid sequence of SEQ ID NO: 1 or SEQ ID NO: B.

8. The nucleic acid molecule of claim 1, wherein the CAR comprises the amino acid sequence of SEQ ID NO: C.

9. The nucleic acid molecule of claim 1, wherein the transmembrane domain is selected from: a CD4 transmembrane domain, a CD8 transmembrane domain, a CD28 transmembrane domain, and a NKG2D transmembrane domain.

10. The nucleic acid molecule of claim 1, wherein the transmembrane domain is a CD28 transmembrane domain or a CD8 transmembrane domain.

11. The nucleic acid molecule of claim 1, wherein the costimulatory domain is a CD28, a 4-1BB, or a 2B4 costimulatory domain.

12. The nucleic acid molecule of claim 1, wherein the costimulatory domain comprises the amino acid sequence of any of SEQ ID NOs:22-25 and 66.

13. The nucleic acid molecule of claim 1, wherein the CD3ζ signaling domain comprises the amino acid sequence of SEQ ID NO:21 or a variant thereof comprising any of SEQ ID NOs: 50-56.

14. The nucleic acid molecule of claim 1, wherein a linker of 3 to 15 amino acids is located between the costimulatory domain and the CD3ζ signaling domain or variant thereof.

15. The nucleic acid molecule of claim 1, wherein the spacer comprises any one of SEQ ID NOs:2-12 and 44.

16. The nucleic acid molecule of claim 1, wherein the targeting domain comprises an scFv targeted to any cancer cell antigen.

17. The nucleic acid molecule of claim 16, wherein the scFv is target to any one or more of CD19, MUC16, MUC1 (or tMUC1), CAIX, CEA, CD20, CD22, CD30, HER-2, ERBB2, MAGEA3, p53, PSCA BCMA, CD123, CD44V6, Integrin B7, TCAM-1, CD70, CEA, GD2, PSMA, B7H3, CD33, Flt3, CLL1, folate receptor, EGFR, CD7, EGFRvIII, glypican3, CD5, ROR1, CS1, AFP, CD133, and TAG-72.

18. The nucleic acid molecule of claim 1, wherein the targeting domain comprises a ligand.

19. The nucleic acid of claim 18, wherein the ligand is selected from an IL-13 or a variant thereof, a chlorotoxin or variant thereof.

20. The nucleic acid molecule of claim 1, wherein the CAR comprises the amino acid sequence of any of SEQ ID NOs 70-76, or a variant thereof having 1-5 amino acid modifications.

21. The nucleic acid molecule of claim 1, wherein the polypeptide comprising human interferon gamma comprises a signal sequence for secretion of human interferon gamma.

22. The nucleic acid molecule of claim 1, wherein the polypeptide comprising human interferon gamma comprises a signal sequence for secretion of human interferon gamma that differs from the native human interferon gamma signal sequence.

23. A population of human T cells harboring: (a) a nucleic acid molecule comprising a nucleotide sequence encoding a chimeric antigen receptor (CAR), wherein the chimeric antigen receptor or polypeptide comprises: a targeting domain, a spacer, a transmembrane domain, a co-stimulatory domain, and a CD3ζ signaling domain; and a nucleotide sequence encoding a polypeptide comprising a human interferon gamma or a variant thereof
Or (b) a nucleic acid molecule comprising a nucleotide sequence encoding a chimeric antigen receptor (CAR), wherein the chimeric antigen receptor or polypeptide comprises: a targeting domain, a spacer, a transmembrane domain, a co-stimulatory domain, and a CD3ζ signaling domain; and a nucleic acid molecule comprising a nucleotide sequence encoding a polypeptide comprising a human interferon gamma or a variant thereof.

24. A population of human T cells harboring: (a) a nucleic acid molecule comprising a nucleotide sequence encoding a chimeric antigen receptor (CAR), wherein the chimeric antigen receptor or polypeptide comprises: a targeting domain, a spacer, a transmembrane domain, a co-stimulatory domain, and a CD3ζ signaling domain; and a nucleotide sequence encoding a polypeptide comprising a human interferon gamma or a variant thereof
Or (b) a nucleic acid molecule comprising a nucleotide sequence encoding a chimeric antigen receptor (CAR), wherein the chimeric antigen receptor or polypeptide comprises: a targeting domain, a spacer, a transmembrane domain, a co-stimulatory domain, and a CD3ζ signaling domain; and a nucleic acid molecule comprising a nucleotide sequence encoding a polypeptide comprising a human interferon gamma or a variant thereof.

25. The population of human T cells of claim 23 or 24, wherein the nucleic acid molecule comprises a promoter that controls expression of both the CAR and human interferon gamma.

26. The population of human T cells of claim 23 or 24, wherein the nucleic acid molecule comprises a promoter that controls expression of both the CAR and human interferon gamma.

27. The population of human T cells of claim 23 or 24, wherein a first promoter controls expression of the CAR and a second promoter controls expression of the human interferon gamma or variant thereof.

28. The population of human T cells of claim 27, wherein the first promoter is a constitutive promoter and the second promoter is a constitutive promoter or is an inducible promoter.

29. The population of human T cells of claim 23 or 24, where a nucleotide sequence encoding a 2A skip sequence is located between the nucleotide sequence encoding a CAR and the nucleotide sequence encoding a human interferon gamma or a variant thereof.

30. The population of human T cells of claim 23 or 24, wherein the 2A skip sequence is selected from the group consisting of T2A, P2A, E2A and F2A.

31. The population of human T cells of claim 23 or 24, wherein the human interferon gamma comprises the amino acid sequence of SEQ ID NO: 1 or SEQ ID NO: B.

32. The population of human T cells of claim 23 or 24, wherein the CAR comprises the amino acid sequence of SEQ ID NO: C.

33. The population of human T cells of claim 23 or 24, wherein the transmembrane domain is selected from: a CD4 transmembrane domain, a CD8 transmembrane domain, a CD28 transmembrane domain, and a NKG2D transmembrane domain.

34. The population of human T cells of claim 23 or 24, wherein the transmembrane domain is a CD28 transmembrane domain or a CD8 transmembrane domain.

35. The population of human T cells of claim 23 or 24, wherein the costimulatory domain is a CD28, a 4-1BB, or a 2B4 costimulatory domain.

36. The population of human T cells of claim 23 or 24, wherein the costimulatory domain comprises the amino acid sequence of any of SEQ ID NOs:22-25 and 66.

37. The population of human T cells of claim 23 or 24, wherein the CD3′ signaling domain comprises the amino acid sequence of SEQ ID NO:21 or a variant thereof comprising any of SEQ ID NOs: 50-56.

38. The population of human T cells of claim 23 or 24, wherein a linker of 3 to 15 amino acids is located between the costimulatory domain and the CD3ζ signaling domain or variant thereof.

39. The population of human T cells of claim 23 or 24, wherein the spacer comprises any one of SEQ ID NOs:2-12 and 44.

40. The population of human T cells of claim 23 or 24, wherein the targeting domain comprises an scFv targeted to any cancer cell antigen.

41. The population of human T cells of claim 40, wherein the scFv is target to any one or more of CD19, MUC16, MUC1 (or tMUC1), CAIX, CEA, CD20, CD22, CD30, HER-2, MAGEA3, p53, PSCA BCMA, CD123, CD44V6, Integrin B7, ICAM-1, CD70, CEA, GD2, PSMA, B7H3, CD33, Flt3, CLL1, folate receptor, EGFR, CD7, EGFRvIII, glypican3, CD5, ROR1, CS1, AFP, CD133, and TAG-72.

42. The population of human T cells of claim 23 or 24, wherein the targeting domain comprises a ligand.

43. The population of human T cells of claim 42, wherein the ligand is selected from an IL-13 or a variant thereof, a chlorotoxin or variant thereof.

44. The population of human T cells of claim 23 or 24, wherein the CAR comprises the amino acid sequence of any of SEQ ID NOs 70-76, or a variant thereof having 1-5 amino acid modifications.

45. The population of human T cells of claim 23 or 24, wherein the polypeptide comprising human interferon gamma comprises a signal sequence for secretion of human interferon gamma.

46. The population of human T cells of claim 23 or 24, wherein the polypeptide comprising human interferon gamma comprises a signal sequence for secretion of human interferon gamma that differs from the native human interferon gamma signal sequence.

47. A method of treating a cancer in a patient comprising administering a population of autologous or allogeneic human T cells transduced by a vector comprising the nucleic acid molecule of any one of claims 1-22, wherein the cancer is targeted by the targeting domain of the CAR.

48. A method of treating a cancer in a patient comprising administering the population of human T cells of claims 23-46, wherein the human T cells are autologous or allogeneic, and wherein the cancer is targeted by the targeting domain of the CAR.

49. The population of human T cells of claim 28, wherein the first promoter is a strong constitutive promoter and the second promoter is a weaker constitutive promoter.

50. The nucleic acid molecule of claim 4, wherein the first promoter is a strong constitutive promoter and the second promoter is a weaker constitutive promoter.


SEQUENCE LISTING (108 sequences)

SEQ ID NO: 1 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 2 - protein, 10 aa, Artificial Sequence
  Description of Artificial Sequence: Synthetic peptide
GGGSSGGGSG

SEQ ID NO: 3 - protein, 12 aa, Artificial Sequence
  Description of Artificial Sequence: Synthetic peptide
ESKYGPPCPPCP

SEQ ID NO: 4 - protein, 12 aa, Artificial Sequence
  Description of Artificial Sequence: Synthetic peptide
ESKYGPPCPSCP

SEQ ID NO: 5 - protein, 22 aa, Artificial Sequence
  Description of Artificial Sequence: Synthetic peptide
ESKYGPPCPPCPGGGSSGGGSG

SEQ ID NO: 6 - protein, 39 aa, Artificial Sequence
  Description of Artificial Sequence: Synthetic polypeptide
IEVMYPPPYLDNEKSNGTIIHVKGKHLCPSPLFPGPSKP

SEQ ID NO: 7 - protein, 48 aa, Artificial Sequence
  Description of Artificial Sequence: Synthetic polypeptide
AKPTTTPAPRPPTPAPTIASQPLSLRPEACRPAAGGAVHTRGLDFACD

SEQ ID NO: 8 - protein, 45 aa, Artificial Sequence
  Description of Artificial Sequence: Synthetic polypeptide
TTTPAPRPPTPAPTIASQPLSLRPEACRPAAGGAVHTRGLDFACD

SEQ ID NO: 9 - protein, 129 aa, Artificial Sequence
  Description of Artificial Sequence: Synthetic polypeptide
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, Artificial Sequence
  Description of Artificial Sequence: Synthetic polypeptide
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, Artificial Sequence
  Description of Artificial Sequence: Synthetic polypeptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence
  Description of Artificial Sequence: Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Artificial Sequence
  Description of Artificial Sequence: Synthetic polypeptide
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 323 aa, Artificial Sequence
  Description of Artificial Sequence: Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 27 - protein, 24 aa, Artificial Sequence
  Description of Artificial Sequence: Synthetic peptide
LEGGGEGRGSLLTCGDVEENPGPR

SEQ ID NO: 28 - protein, 354 aa, Artificial Sequence
  Description of Artificial Sequence: Synthetic polypeptide
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 30 - protein, 115 aa, Artificial Sequence
  Description of Artificial Sequence: Synthetic polypeptide
GPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLLHLKKLFREGRFN

SEQ ID NO: 31 - protein, 146 aa, Homo sapiens [sequence omitted]

SEQ ID NO: 32 - protein, 133 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQ

SEQ ID NO: 33 - protein, 282 aa, Artificial Sequence
  Description of Artificial Sequence: Synthetic polypeptide
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNK

SEQ ID NO: 34 - DNA, 30 bp, Artificial Sequence
  Description of Artificial Sequence: Synthetic oligonucleotide
tggaggaaaaactgtttcatacagaaggcg

SEQ ID NO: 35 - DNA, 60 bp, Artificial Sequence
  Description of Artificial Sequence: Synthetic oligonucleotide
tggaggaaaaactgtttcatacagaaggcgtggaggaaaaactgtttcatacagaaggcg

SEQ ID NO: 36 - DNA, 90 bp, Artificial Sequence
  Description of Artificial Sequence: Synthetic oligonucleotide
tggaggaaaaactgtttcatacagaaggcgtggaggaaaaactgtttcatacagaaggcgtggaggaaaaactgtttcatacagaaggcg

SEQ ID NO: 37 - DNA, 120 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 38 - DNA, 150 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 39 - DNA, 180 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 40 - DNA, 210 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 41 - DNA, 240 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 42 - DNA, 270 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 43 - DNA, 300 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 44 - protein, 16 aa, Artificial Sequence
  Description of Artificial Sequence: Synthetic peptide
LEPKSCDKTHTCPPCP

SEQ ID NO: 45 - protein, 357 aa, Artificial Sequence
  Description of Artificial Sequence: Synthetic polypeptide
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 46 - protein, 18 aa, Artificial Sequence
  Description of Artificial Sequence: Synthetic peptide
EGRGSLLTCGDVEENPGP

SEQ ID NO: 47 - protein, 22 aa, Artificial Sequence
  Description of Artificial Sequence: Synthetic peptide
GSGATNFSLLKQAGDVEENPGP

SEQ ID NO: 48 - DNA, 330 bp, Artificial Sequence
  Description of Artificial Sequence: Synthetic polynucleotide
tggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcgtggaggaaaaactgtttcatacagaaggcg

SEQ ID NO: 49 - skipped

SEQ ID NO: 50 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 51 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTYDALHMQALPPR

SEQ ID NO: 52 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 53 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLYNELQKDKMAEAFSEIGMKGERRRGKGHDGLFQGLSTATKDTFDALHMQALPPR

SEQ ID NO: 54 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLFNELQKDKMAEAYSEIGMKGERRRGKGHDGLFQGLSTATKDTFDALHMQALPPR

SEQ ID NO: 55 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLYQGLSTATKDTFDALHMQALPPR

SEQ ID NO: 56 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTYDALHMQALPPR

SEQ ID NO: 57-64 - skipped (8 entries)

SEQ ID NO: 65 - protein, 21 aa, Homo sapiens
PFFFCCFIAVAMGIRFIIMVA

SEQ ID NO: 66 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 67-69 - skipped (3 entries)

SEQ ID NO: 70 - protein, 525 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 520 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 518 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 402 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 398 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 396 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIYIWAPLAGTCGVLLLSLVITKRGRKKLLYIFKQPFMRPVQTTQEEDGCSCRFPEEEEGGCELGGGRVKFSRSADAPAYQQGQNQLYNELNLGRREYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 520 aa, Artificial Sequence
  Description of Artificial Sequence: Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - DNA, 18 bp, Artificial Sequence
  Description of Artificial Sequence: Synthetic primer
cctcgcctttgccgatcc

SEQ ID NO: 78 - DNA, 23 bp, Artificial Sequence
  Description of Artificial Sequence: Synthetic primer
ggatcttcatgaggtagtcagtc

SEQ ID NO: 79 - DNA, 21 bp, Artificial Sequence
  Description of Artificial Sequence: Synthetic primer
aaactcgcatctactggcaaa

SEQ ID NO: 80 - DNA, 21 bp, Artificial Sequence
  Description of Artificial Sequence: Synthetic primer
ggttcttgtactcgggccata

SEQ ID NO: 81 - DNA, 23 bp, Artificial Sequence
  Description of Artificial Sequence: Synthetic primer
ctgctcatctatacacggttacc

SEQ ID NO: 82 - DNA, 22 bp, Artificial Sequence
  Description of Artificial Sequence: Synthetic primer
ggaaacgtcgtacagttctgtg

SEQ ID NO: 83 - DNA, 21 bp, Artificial Sequence
  Description of Artificial Sequence: Synthetic primer
gtggcattcaaggagtacctc

SEQ ID NO: 84 - DNA, 22 bp, Artificial Sequence
  Description of Artificial Sequence: Synthetic primer
tgatggccttcgattctggatt

SEQ ID NO: 85 - DNA, 21 bp, Artificial Sequence
  Description of Artificial Sequence: Synthetic primer
ccagtagtgagaaagggtcgc

SEQ ID NO: 86 - DNA, 20 bp, Artificial Sequence
  Description of Artificial Sequence: Synthetic primer
agggcttggggcaaattgtt

SEQ ID NO: 87 - DNA, 21 bp, Artificial Sequence
  Description of Artificial Sequence: Synthetic primer
ggttgtggtaaacctctgctc

SEQ ID NO: 88 - DNA, 20 bp, Artificial Sequence
  Description of Artificial Sequence: Synthetic primer
aaagctggtgccgttgagaa

SEQ ID NO: 89 - DNA, 21 bp, Artificial Sequence
  Description of Artificial Sequence: Synthetic primer
ctcatgaccacagtccatgcc

SEQ ID NO: 90 - DNA, 20 bp, Artificial Sequence
  Description of Artificial Sequence: Synthetic primer
aggccatgccagtgagcttc

SEQ ID NO: 91 - DNA, 20 bp, Artificial Sequence
  Description of Artificial Sequence: Synthetic primer
gagccaccactcaccctact

SEQ ID NO: 92 - DNA, 19 bp, Artificial Sequence
  Description of Artificial Sequence: Synthetic primer
ccaggcattcggcaatgtg

SEQ ID NO: 93 - DNA, 20 bp, Artificial Sequence
  Description of Artificial Sequence: Synthetic primer
gtgcagaagagagcgatccg

SEQ ID NO: 94 - DNA, 20 bp, Artificial Sequence
  Description of Artificial Sequence: Synthetic primer
cggttagacccccatagtgc

SEQ ID NO: 95 - DNA, 19 bp, Artificial Sequence
  Description of Artificial Sequence: Synthetic primer
gctgggcatcgttgttgga

SEQ ID NO: 96 - DNA, 23 bp, Artificial Sequence
  Description of Artificial Sequence: Synthetic primer
gcactttgtaggatagcaggaag

SEQ ID NO: 97 - DNA, 21 bp, Artificial Sequence
  Description of Artificial Sequence: Synthetic primer
agctcattgtagaaggtgtgg

SEQ ID NO: 98 - DNA, 19 bp, Artificial Sequence
  Description of Artificial Sequence: Synthetic primer
gtgggaatgggtcagaagg

SEQ ID NO: 99 - DNA, 21 bp, Artificial Sequence
  Description of Artificial Sequence: Synthetic primer
gggtaccatgaagatctctgc

SEQ ID NO: 100 - DNA, 21 bp, Artificial Sequence
  Description of Artificial Sequence: Synthetic primer
tctagggagaggtaggcaaag

SEQ ID NO: 101 - DNA, 20 bp, Artificial Sequence
  Description of Artificial Sequence: Synthetic primer
aaccgaagtcatagccacac

SEQ ID NO: 102 - DNA, 18 bp, Artificial Sequence
  Description of Artificial Sequence: Synthetic primer
cagacggtgccatcagag

SEQ ID NO: 103 - DNA, 18 bp, Artificial Sequence
  Description of Artificial Sequence: Synthetic primer
tcagcaccatgaacccaa

SEQ ID NO: 104 - DNA, 21 bp, Artificial Sequence
  Description of Artificial Sequence: Synthetic primer
ctatggccctcattctcactg

SEQ ID NO: 105 - DNA, 22 bp, Artificial Sequence
  Description of Artificial Sequence: Synthetic primer
gaagtcatagccactctcaagg

SEQ ID NO: 106 - DNA, 18 bp, Artificial Sequence
  Description of Artificial Sequence: Synthetic primer
cttccgttgagggacagc

SEQ ID NO: 107 - DNA, 23 bp, Artificial Sequence
  Description of Artificial Sequence: Synthetic primer
gttctcagcccaacaatacaaga

SEQ ID NO: 108 - DNA, 19 bp, Artificial Sequence
  Description of Artificial Sequence: Synthetic primer
gtggacgggtcgatgtcac